(19) United States
(12) Patent Application Publication
(10) Pub. No.: US 2024/0390410 A1
(43) Pub. Date: Nov. 28, 2024

(54) MICRORNA-29 COMPOUNDS, COMPOSITIONS AND USES IN THERAPY

(71) Applicant: Causeway Therapeutics Limited, Glasgow (GB)

(72) Inventors: Derek Steward Gilchrist, Glasgow (GB); Neal Lindsay MILLAR, Glasgow (GB)

(21) Appl. No.: 18/033,322

(22) PCT Filed: Oct. 25, 2021
(86) PCT No.: PCT/EP2021/079580

§ 371 Date: Apr. 21, 2023

(30) Foreign Application Priority Data
Oct. 23, 2020 (GB) 2016863.9

(51) Int. Cl. A61K 31/713; C12N 15/113

(52) U.S. Cl. CPC A61K 31/713; C12N 15/113; C12N 2310/141; C12N 2310/321; C12N 2310/322; C12N 2310/3515; C12N 2320/35

(57) ABSTRACT

This invention relates to novel microRNA-29 (miR-29) compounds, compositions comprising the same, and their use in therapy. In particular, provided herein are miR-29 compounds and compositions that are particularly effective and safe in the treatment of diseases that involve localised collagen dysregulation, such as tendon damage including tendinopathy, and tissue fibrosis including Peyronie's disease and Dupuytren's disease. Also provided are advantageous dosages and treatment regimens for miR-29 compounds.

### CROSS-REFERENCE TO RELATED APPLICATION

This application is a U.S. National Stage Application under 35 U.S.C. 371 of International Patent Application No. PCT/EP2021/079580, filed on Oct. 25, 2021, which claims priority to Application Number GB2016863.9, filed on Oct. 23, 2020, each of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Apr. 21, 2023, is named 350647_ST25.txt and is 1,900 bytes in size.

### TECHNICAL FIELD

This invention relates to novel microRNA-29 (miR-29) compounds, compositions comprising the same, and their use in therapy. In particular, provided herein are miR-29 compounds and compositions that are particularly effective and safe in the treatment of diseases that involve localised collagen dysregulation, such as tendon damage including tendinopathy, and tissue fibrosis including Peyronie's disease and Dupuytren's disease. Also provided are advantageous dosages and treatment regimens for miR-29 compounds.

### BACKGROUND AND SUMMARY

MicroRNAs (miRs) are small, non-coding RNAs that work alongside the RNA-Induced Silencing Complex (RISC) to reduce or prevent the translation of messenger RNAs (mRNA) containing complementary target sequences.

Members of the miR-29 family have been implicated as post-transcriptional regulators of the extracellular matrix, e.g., collagen. Dysregulated collagen synthesis, in particular the overproduction of one or more types of collagen, contributes to the pathology seen in certain diseases, such as tendon damage including tendinopathy, and tissue fibrosis including Peyronie's disease and Dupuytren's disease.

Previously published studies by the inventors (refs. 1, 2, 3) established that miR-29 regulates key disease pathways in collagen production in tendon damage. For example, in healthy tenocytes, miR-29 (e.g., miR-29a, also referred herein as miR29a) suppresses collagen 3 (col3) overproduction, whereas in tendon disease there is a reduction of miR-29 levels and an overproduction of col3, which leads to mechanically inferior tendon tissue that is prone to further damage. In those studies the inventors showed that in vitro transfection of tenocytes with a miR-29a compound significantly decreased col3 expression, whereas the levels of col1, i.e., the desirable and biomechanically superior form of collagen in tendon, remained largely unaffected. The inventors also demonstrated that the majority of col1 transcripts in tenocytes lacked a binding site for miR-29 due to the utilisation of an alternative polyadenylation site, whereas the majority of col3 transcripts in tenocytes retained a miR-29 binding site. Col1 transcripts in tenocytes were therefore rendered insensitive to miR-29 mediated downregulation, whereas miR-29 was capable of specifically suppressing col3 in those cells. This was confirmed in in vivo models of tendon injury, where it was shown that delivery of miR-29a compounds to injured tendon of mice (refs. 1, 2) or horses (ref. 3) resulted in a reduction of col3 synthesis, while maintaining col1 levels. Previous studies had shown that in fibroblasts miR-29 inhibited the expression of not only col3 but also other types of collagen, including col1 (e.g., ref. 4).

The present invention provides improved miR-29 compounds that are particularly effective and safe in therapy. These compounds are useful in the treatment of any condition that requires increased miR-29 (e.g., miR-29a) activity. This encompasses conditions that involve localised collagen dysregulation and require reducing or inhibiting expression of one or more types of collagen, such as collagen 3, including but not limited to tendon damage including tendinopathy (e.g., lateral epicondylitis, also known as tennis elbow), and tissue fibrosis such as Peyronie's disease and Dupuytren's disease. The compounds described herein mimic the activity of natural miR-29 while certain chemical modifications provide improved pharmacological properties, such as increased activity, stability and cellular uptake, yet without causing immunogenicity issues. A further advantage of the compounds and compositions described herein is that they are stable in a simple aqueous carrier and can directly be administered to the afflicted site without the need for reconstitution or dilution.

Provided herein are also particularly advantageous dosages and treatment regimens for miR-29 compounds in the treatment of diseases such as tendon damage including tendinopathy, and tissue fibrosis such as Peyronie's disease and Dupuytren's disease. The inventors have shown that administering by local (intra-tendon) injection of a miR-29 compound to a damaged tendon improves tendon healing and tendon fibre quality, e.g., in a mouse model of tendinopathy. Surprisingly, therapeutic efficacy can be achieved with only a single administration (i.e., a single dose). Dosages suitable for therapy (e.g., human therapy) are provided. Data are also available to support therapeutic efficacy of miR-29 compounds in tissue fibrosis such as Dupuytren's and Peyronie's disease. The miR-29 compounds can be administered locally at the site of the pathology (intralesionally) in a simple aqueous carrier. Data provided herein show that there is negligible systemic exposure or distribution into other tissues following local intralesional administration, thereby providing a particularly safe therapy. Furthermore, because a single dose can be therapeutically effective, only one visit to a healthcare professional is required, thereby enabling a simple, cost-effective and fast yet effective treatment.

### DISCLOSURE OF THE INVENTION

In one aspect, the present invention provides miR-29 compounds, compositions comprising the same, and their use in therapy. The invention therefore provides a miR-29 compound or a salt thereof comprising a guide (antisense) strand and a passenger (sense) strand, which mimics the biological activity of native miR-29a, for example the downregulation of collagen 3 expression. The compounds of the invention are therefore capable of reducing or inhibiting collagen 3 expression. As the biological activity between the various members of the miR-29 family (miR-29a, miR-29b-1, miR-29b-2 and miR-29c) overlap, the miR-29 compounds provided herein can also mimic the biological activity of the other miR-29 family members. The miR-29 compound can therefore also be referred to as a miR-29 mimic, or a miR-29 mimetic compound.

The guide and passenger strands of the miR-29 compounds are oligonucleotides, i.e., they contain nucleosides linked by internucleoside linkages. The guide and passenger strands form a duplex, typically containing a number of mismatches and an overhang in at least one strand. The miR-29 compounds described herein are typically chemically modified, which means that at least one sugar, internucleoside linkage or nucleobase has a chemical structure that differs from that in naturally-occurring miR-29. The chemical modification(s) can be selected to improve the pharmacological properties of the miR-29 compound, e.g., by enhancing its activity and/or stability. Exemplary modified sugars are 2′-fluoro ribose, 2′-O-methyl ribose or 2′-deoxy ribose. An exemplary modified internucleoside linkage is a phosphorothioate internucleoside linkage. An exemplary modified nucleobase is a 5-methyl cytosine. The miR-29 compounds may also be conjugated (i.e., covalently linked) to a lipophilic moiety such as a cholesterol moiety, typically at the 3′ end of the passenger strand. Such conjugation to a lipophilic moiety may improve the properties of the miR-29 compound for in vivo use, e.g., by improving its cellular uptake.

In one embodiment, the invention therefore provides a miR-29 compound or a salt thereof comprising:
(a) a guide strand comprising a nucleobase sequence which differs from 5′-UAGCACCAUCUGAAAUCGGUUA-3′ (SEQ ID NO: 1) at no more than five (or no more than four, three, two, or one) positions outside of the sequence 5′-AGCACCA-3′ (SEQ ID NO: 8, which is the seed sequence), and optionally at no more than three (or no more than two, or one) positions within the seed sequence; wherein at least one nucleoside comprises a 2′-fluoro ribose and optionally wherein at least one nucleoside comprises a 2′-O-methyl ribose; and
(b) a passenger strand, comprising a nucleobase sequence that differs from 5′-ACUGAUUUCUUUUGGUGUUCAG-3′ (SEQ ID NO: 2) at no more than five (or no more than four, three, two, or one; for example at no more than three) positions; and wherein at least one nucleoside comprises a 2′-O-methyl ribose and at least one nucleoside comprises an unmodified ribose.

A nucleobase sequence “differs” from another nucleobase sequence if there are different, additional, or missing nucleobases in corresponding positions. A nucleobase sequence that differs from another nucleobase sequence at no more than a certain number of positions also encompasses a nucleobase sequence that differs at zero positions, i.e., the nucleobase sequences may be identical.

Thus the nucleobase sequence of the guide strand may differ from the nucleobase sequence of native miR-29a (SEQ ID NO: 1) outside of the seed sequence of native miR-29a (AGCACCA, SEQ ID NO: 8) at no more than five positions, e.g., at no more than four positions, no more than three positions, no more than two positions, or at no more than one position, preferably at no more than three positions.

In one embodiment the nucleobase sequence of the guide strand outside of the seed sequence is identical to the corresponding positions in SEQ ID NO: 1. Additionally or alternatively, the seed sequence of the guide strand of the miR-29 compound may differ from the seed sequence of native miR-29a (AGCACCA, SEQ ID NO: 8) at no more than three positions, e.g., at no more than two positions, or at no more than one position. In a preferred embodiment the seed sequence is identical to SEQ ID NO: 8.

The nucleobase sequence of the passenger strand may differ from 5′-ACUGAUUUCUUUUGGUGUUCAG-3′ (SEQ ID NO: 2) at no more than five positions, for example at no more than four positions. In preferred embodiments, the nucleobase sequence of the passenger strand differs from SEQ ID NO: 2 at no more than three positions. In other embodiments, the nucleobase sequence of the passenger strand differs from SEQ ID NO: 2 at no more than two positions. In a specific embodiment, the nucleobase sequence of the passenger strand differs from SEQ ID NO: 2 at no more than one position. This means that the nucleobase sequence of the passenger strand has the sequence of SEQ ID NO: 2 or a sequence that has one different, additional, or missing nucleobase relative to SEQ ID NO: 2. SEQ ID NO: 3 has one missing nucleobase relative to SEQ ID NO: 2.

As SEQ ID NO: 2 and SEQ ID NO: 3 differ only in one position, also provided herein are embodiments wherein the passenger strand differs from SEQ ID NO: 3 in no more than five etc. positions, as above.

In certain embodiments, the passenger strand comprises a nucleobase sequence that differs from 5′-ACUGAUUUCUUUUGGUGUUCAG-3′ (SEQ ID NO: 2) at no more than five (e.g., no more than four, three, two, or one; such as no more than three) positions outside of the sequence 5′-UUU-3′ and/or 5′-UUCA-3′ underlined in SEQ ID NO: 2 above, which correspond to positions 10-12 and 18-21 of SEQ ID NO: 2. In certain embodiments, the nucleobase sequence of the passenger strand differs from SEQ ID NO: 2 only outside of the sequences 5′-UUU-3′ and 5′-UUCA-3′. For example, the nucleobase sequence of the passenger strand may differ from SEQ ID NO: 2 at no more than two positions outside of the sequences 5′-UUU-3′ and 5′-UUCA-3′. In a specific embodiment, the nucleobase sequence of the passenger strand may differ from SEQ ID NO: 2 at no more than one or two positions outside of the sequences 5′-UUU-3′ and 5′-UUCA-3′, for example at no more than one position. In specific embodiments, such a passenger strand is combined with a fluorinated guide strand as described herein (e.g., comprising one or more 2′-fluoro ribonucleosides, such as wherein at least 50% of the nucleosides are 2′-fluoro ribonucleosides, or wherein all nucleosides except one or two 2′-O-methyl ribose nucleosides, preferably located at the 3′ end, are 2′-fluoro ribonucleosides).

In one embodiment, the guide strand has a nucleobase sequence comprising at least 7 consecutive nucleobases of the nucleobase sequence of SEQ ID NO: 1. For example, the guide strand may have a nucleobase sequence comprising at least 8, 9, 10, 11, 12, 13, 14, 15, 16, 17, 18, 19, 20 or at least 21 consecutive nucleobases of the nucleobase sequence of SEQ ID NO: 1. In one embodiment, the guide strand has a nucleobase sequence comprising at least 18 consecutive nucleobases of the nucleobase sequence of SEQ ID NO: 1.

In the same or other embodiments, the passenger strand may have a nucleobase sequence comprising at least 7 consecutive nucleobases of the nucleobase sequence of SEQ ID NO: 2. For example, the passenger strand may have a nucleobase sequence comprising at least 8, 9, 10, 11, 12, 13, 14, 15, 16, 17, 18, 19, 20 or at least 21 consecutive nucleobases of the nucleobase sequence of SEQ ID NO: 2. In one embodiment, the passenger strand has a nucleobase sequence comprising at least 18 consecutive nucleobases of the nucleobase sequence of SEQ ID NO: 2.

In one embodiment, the guide strand comprises a nucleobase sequence that is at least 85% identical across the entire length of SEQ ID NO: 1. For example, the guide strand may comprise a nucleobase sequence that is at least 90%, 95%, 96%, 97%, 98%, at least 99%, or 100% identical across the entire length of SEQ ID NO: 1. In certain embodiments, the guide strand has a nucleobase sequence as defined in the preceding sentence and it comprises the sequence 5′-AGCACCA-3′ (SEQ ID NO: 8) at positions that correspond to positions 2-8 of SEQ ID NO: 1. In those embodiments, the nucleobase sequence of the guide strand may differ from SEQ ID NO: 1 only outside of positions 2-8 of SEQ ID NO: 1. The difference may be defined by percentage identity or by the number of identical/different nucleobases, as described above.

In the same or other embodiments, the passenger strand may comprise a nucleobase sequence that is at least 85% identical across the entire length of SEQ ID NO: 2 or 3. For example, the passenger strand may comprise a nucleobase sequence that is at least 90%, 95%, 96%, 97%, 98%, at least 99%, or 100% identical across the entire length of SEQ ID NO: 2 or 3. In certain embodiments, the passenger strand has a nucleobase sequence as defined in the preceding sentence and it comprises the sequence 5′-UUU-3′ and/or 5′-UUCA-3′ at positions that correspond to positions 10-12 and 18-21 of SEQ ID NO: 2. In those embodiments, the nucleobase sequence of the passenger strand may differ from SEQ ID NO: 2 only outside of positions 10-12 and/or 18-21 of SEQ ID NO: 2. In a specific embodiment, the nucleobase sequence of the passenger strand may differ from SEQ ID NO: 2 only outside of positions 10-12 and 18-21 of SEQ ID NO: 2. The difference may be defined by percentage identity or by the number of identical/different nucleobases, as described above. In specific embodiments, such a passenger strand is combined with a fluorinated guide strand as described herein (e.g., comprising one or more 2′-fluoro ribonucleosides, such as wherein at least 50% of the nucleosides are 2′-fluoro ribonucleosides, or wherein all nucleosides except one or two 2′-O-methyl ribose nucleosides, preferably located at the 3′ end, are a 2′-fluoro ribonucleosides).

In a further embodiment, the guide strand and/or the passenger strand may consist of the nucleobase sequence as indicated above. In a specific embodiment, the guide strand comprises or consists of the nucleobase sequence 5′-UAGCACCAUCUGAAAUCGGUUA-3′ (SEQ ID NO: 1), and/or the passenger strand comprises or consists of the nucleobase sequence 5′-ACUGAUUUCUUUUGGUGUUCAG-3′ (SEQ ID NO: 2) or 5′-ACUGAUUUCUUUUGGUGUUCA-3′ (SEQ ID NO: 3). In a specific embodiment, the guide strand comprises or consists of the nucleobase sequence 5′-UAGCACCAUCUGAAAUCGGUUA-3′ (SEQ ID NO: 1), and/or the passenger strand comprises or consists of the nucleobase sequence 5′-ACUGAUUUCUUUUGGUGUUCA-3′ (SEQ ID NO: 3). In a specific embodiment, the guide strand comprises nucleobase sequence 5′-UAGCACCAUCUGAAAUCGGUUA-3′ (SEQ ID NO: 1), and the passenger strand comprises the nucleobase sequence 5′-ACUGAUUUCUUUUGGUGUUCA-3′ (SEQ ID NO: 3). In a specific embodiment, the guide strand consists of the nucleobase sequence 5′-UAGCACCAUCUGAAAUCGGUUA-3′ (SEQ ID NO: 1), and the passenger strand consists of the nucleobase sequence 5′-ACUGAUUUCUUUUGGUGUUCA-3′ (SEQ ID NO: 3). In any of these embodiments, the term “consists of” does not exclude the presence of other (non-nucleoside) moieties attached to the guide strand or to the passenger strand, e.g., the passenger strand may be linked to a lipophilic moiety such as a cholesterol moiety, for example via a linker, typically at the 3′ end of the passenger strand.

In certain embodiments, the nucleobase at the 3′ end of the passenger strand is an A or a G. In certain embodiments, the nucleobase at the 3′ end of the passenger strand is an A.

In any of the embodiments described herein, at least or about 1, 2, 3, 4, 5, 6, 7, 8, 9, 10, 11, 12, 13, 14, 15, 16, 17, 18, 19, 20 or 21 nucleosides of the guide strand may comprises a 2′-fluoro ribose. For example, at least or about 11, 12, 13, 14, 15, 16, 17, 18 or 19 nucleosides of the guide strand are 2′-fluoro ribose nucleosides. In certain embodiments, no more than 20 or no more than 21 nucleosides of the guide strand are 2′-fluoro ribose nucleosides.

In any of the embodiments described herein, at least 10%, 20%, 30%, 40%, 50%, 60%, 70%, 80%, 90%, 91%, 92%, 93%, 94% or at least 95% of the nucleosides of the guide strand comprise a 2′-fluoro ribose. In some embodiments, at least one nucleoside of the guide strand contains a sugar (which may be a modified sugar) other than a 2′-fluoro ribose. In a specific embodiment, at least 50% (or at least 60%, 70%, 80%, 90%, 91%, 92%, 93%, 94% or at least 95%) of the nucleosides of the guide strand comprise a 2′-fluoro ribose, and at least one nucleoside of the guide strand comprises a 2′-O-methyl ribose. In another specific embodiment, at least 50% (or at least 60%, 70%, 80%, 90%, 91%, 92%, 93%, 94% or at least 95%) of the nucleosides of the guide strand comprise a 2′-fluoro ribose, and at least two nucleosides of the guide strand comprise a 2′-O-methyl ribose. For example, one or two nucleosides of the guide strand may comprise a 2′-O-methyl ribose. The nucleosides of the guide strand that comprise a 2′-O-methyl ribose may be located at the 3′ end of the guide strand.

In one embodiment, at least 50%, 60%, 70%, 80% or 90% of the nucleosides of the guide strand are 2′-fluoro ribose nucleosides, and/or no more than 91%, or no more than 95%, of the nucleosides of the guide strand are 2′-fluoro ribose nucleosides. For example, at least 50% and no more than 91% of the nucleosides of the guide strand are 2′-fluoro ribose nucleosides.

In one embodiment, the nucleosides of the passenger strand that comprise a 2′-O-methyl ribose and an unmodified ribose alternate. For example, at least 10, 11, 12, 13, 14, 15, 16, 17, 18, 19 or at least 20 contiguous nucleosides in the passenger strand may be alternately a 2′-O-methyl ribose nucleoside and a nucleoside with an unmodified ribose moiety. Thus there may be at least 10 contiguous nucleosides in the passenger strand that are alternately a 2′-O-methyl ribose nucleoside and a nucleoside with an unmodified ribose moiety. There may be at least 12 contiguous nucleosides in the passenger strand that are alternately a 2′-O-methyl ribose nucleoside and a nucleoside with an unmodified ribose moiety. There may be at least 14 contiguous nucleosides in the passenger strand that are alternately a 2′-O-methyl ribose nucleoside and a nucleoside with an unmodified ribose moiety. There may be at least 16 contiguous nucleosides in the passenger strand that are alternately a 2′-O-methyl ribose nucleoside and a nucleoside with an unmodified ribose moiety. In a preferred embodiment, there are at least 18 contiguous nucleosides in the passenger strand that are alternately a 2′-O-methyl ribose nucleoside and a nucleoside with an unmodified ribose moiety. One or more nucleosides of the passenger strand may comprise a deoxy ribose. Such nucleosides may be located at the 3′ end of the passenger strand.

In one embodiment, at least 5, 6, 7, 8 or 9 nucleosides of the passenger strand are 2′-O-methyl ribose nucleosides, and/or no more than 10 nucleosides of the passenger strand are 2′-O-methyl ribose nucleosides.

In one embodiment, at least 10%, 20%, 30%, 40% or 45% of the nucleosides of the passenger strand are 2′-O-methyl ribose nucleotides, and/or no more than 50% of the oligonucleotide of the passenger strand are 2′-O-methyl ribose nucleotides.

The invention thus provides a miR-29 compound or a salt thereof that includes a guide strand as described in any of the embodiments herein, and a passenger strand as described in any of the embodiments herein.

In one embodiment therefore, the invention provides a miR-29 compound or a salt thereof, comprising:
(a) a guide strand comprising the nucleobase sequence 5′-UAGCACCAUCUGAAAUCGGUUA-3′ (SEQ ID NO: 1), or a nucleobase sequence which differs from SEQ ID NO: 1 at no more than five positions outside of the sequence 5′-AGCACCA-3′ (SEQ ID NO: 8), and optionally at no more than three positions within that sequence; wherein at least 50% of the nucleosides comprise a 2′-fluoro ribose and wherein one or two nucleosides, preferably at the 3′ end, each comprise a 2′-O-methyl ribose; and
(b) a passenger strand, comprising the nucleobase sequence 5′-ACUGAUUUCUUUUGGUGUUCAG-3′ (SEQ ID NO: 2) or 5′-ACUGAUUUCUUUUGGUGUUCA-3′ (SEQ ID NO: 3), or comprising a nucleobase sequence that differs from SEQ ID NO: 2 at no more than three positions; and wherein at least 10 contiguous nucleosides are alternately a 2′-O-methyl ribose nucleoside and a nucleoside with an unmodified ribose moiety.

In a specific embodiment, the invention provides a miR-29 compound or a salt thereof, comprising:
(a) a guide strand comprising the nucleobase sequence 5′-UAGCACCAUCUGAAAUCGGUUA-3′ (SEQ ID NO: 1), or a nucleobase sequence which differs from SEQ ID NO: 1 at no more than three positions outside of the sequence 5′-AGCACCA-3′ (SEQ-ID NO: 8); wherein at least 50% of the nucleosides comprise a 2′ fluoro ribose and wherein one or two nucleosides at the 3′ end each comprise a 2′-O-methyl ribose; and
(b) a passenger strand, comprising the nucleobase sequence 5′-ACUGAUUUCUUUUGGUGUUCAG-3′ (SEQ ID NO: 2) or 5′-ACUGAUUUCUUUUGGUGUUCA-3′ (SEQ ID NO: 3), or comprising a nucleobase sequence that differs from SEQ ID NO: 2 at no more than three positions; and wherein at least 10 contiguous nucleosides are alternately a 2′-O-methyl ribose nucleoside and a nucleoside with an unmodified ribose moiety.

Exemplary compounds according to this embodiment are Teno15, Teno18, Teno19, Teno20, Teno29, Teno32, Teno33 and Teno34, which are described in the Examples below.

As noted above, at least 10, 11, 12, 13, 14, 15, 16, 17, 18, 19 or at least 20 contiguous nucleosides in the passenger strand may be alternately a 2′-O-methyl ribose nucleoside and a nucleoside with an unmodified ribose moiety. For example, at least 12 contiguous nucleosides in the passenger strand are alternately a 2′-O-methyl ribose nucleoside and a nucleoside with an unmodified ribose moiety. For example, at least 14 contiguous nucleosides in the passenger strand are alternately a 2′-O-methyl ribose nucleoside and a nucleoside with an unmodified ribose moiety. For example, at least 16 contiguous nucleosides in the passenger strand are alternately a 2′-O-methyl ribose nucleoside and a nucleoside with an unmodified ribose moiety. In certain embodiments, at least 18 contiguous nucleosides in the passenger strand are alternately a 2′-O-methyl ribose nucleoside and a nucleoside with an unmodified ribose moiety.

Preferably the at least one or two nucleosides in the passenger strand that each comprise a 2′-O-methyl ribose are located at the 3′ end of the passenger strand. However, this is not essential.

In certain embodiments, one or two nucleosides at the 3′ end of the guide strand may each comprise a 2′-O-methyl ribose and (i) each remaining nucleoside of the guide strand that comprises a 2′-modified ribose is a 2′-fluoro ribose nucleoside, or (ii) each remaining nucleoside of the guide strand is a 2′-fluoro ribose nucleoside or an unmodified ribose moiety.

In certain embodiments, (i) each nucleoside of the guide strand comprises a 2′-modified ribose, (ii) each nucleoside of the guide strand comprises a 2′-O-methyl ribose or a 2′-fluoro ribose, or (iii) one or two nucleosides at the 3′ end of the guide strand each comprise a 2′-O-methyl ribose and each remaining nucleoside of the guide strand is a 2′-fluoro ribose nucleoside.

In certain embodiments, none of the internucleoside linkages of the guide strand and/or the passenger are chemically modified. Thus, in certain embodiments, all of the internucleoside linkages of the guide strand and/or the passenger strand are phosphodiester internucleoside linkages.

In certain embodiments, none of the nucleobases of the guide strand and/or the passenger are chemically modified. For example, in certain embodiments, none of the nucleobases of the guide strand and/or the passenger strand contain a 5-methyl cytosine.

In certain embodiments, the nucleobase sequence of the guide strand is as defined in SEQ ID NO: 1, and/or the nucleobase sequence of the passenger strand is as defined in SEQ ID NO: 2 or SEQ ID NO: 3.

In certain embodiments, the nucleobase sequence of the guide strand is as defined in SEQ ID NO: 1, and/or the nucleobase sequence of the passenger strand is as defined in SEQ ID NO: 3.

In certain embodiments, the guide strand is 5′-fUfAfGfCfAfCfCfAfUfCfUfGfAfAfAfUfCfGfGfUmUmA-3′; and the passenger is 5′-mArCmUrGmArUmUrUmCrUmUrUmUrGmGrUmGrUmUrCdA-3′; wherein ‘f’ preceding a nucleoside=a 2′-deoxy-fluoro ribonucleoside (—F); ‘m’ preceding a nucleoside=a 2′-O-methyl ribonucleoside (—O-Me); ‘r’ preceding a nucleoside=a nucleoside with an unmodified ribose moiety (—OH); ‘d’ preceding a nucleoside=a 2′-deoxyribonucleoside (—H). An exemplary compound according to this embodiment is Teno18, which is described in the Examples below.

In certain embodiments, the guide strand is 5′-fUfAfGfCfAfCfCfAfUfCfUfGfAfAfAfUfCfGfGfUmUmA-3′; and the passenger is 5′-mAmCrUmGrAmUrUmUrCmUrUmUrUmGrGmUrGmUrUmCmAmG-3′; wherein ‘f’ preceding a nucleoside=a 2′-deoxy-fluoro ribonucleoside (—F); ‘m’ preceding a nucleoside=a 2′-O-methyl ribonucleoside (—O-Me); ‘r’ preceding a nucleoside=a nucleoside with an unmodified ribose moiety (—OH). An exemplary compound according to this embodiment is Teno20, which is described in the Examples below.

In certain embodiments, the guide strand is 5′-fUrArGfCrAfCfCrAfUfCfUrGrArArAfUfCrGrGfUfUmA-3′; and the passenger is 5′-mArCmUrGmArUmUrUmCrUmUrUmUrGmGrUmGrUmUrCmAdG-3′; wherein ‘f’ preceding a nucleoside=a 2′-deoxy-fluoro ribonucleoside (—F); ‘m’ preceding a nucleoside=a 2′-O-methyl ribonucleoside (—O-Me); ‘r’ preceding a nucleoside=a nucleoside with an unmodified ribose moiety (—OH); ‘d’ preceding a nucleoside=a 2′-deoxyribonucleoside (—H). An exemplary compound according to this embodiment is Teno33, which is described in the Examples below.

In any of the embodiments described herein, the miR-29 compound or salt thereof may further comprise a cholesterol moiety covalently attached to the 3′ end of the passenger strand. The cholesterol moiety may be a triethyleneglycol cholesterol moiety. An exemplary cholesterol moiety is 3′phosphopropy-2-ol,2,2′-[Ethane-1,2-diylbis(oxy)]di(ethan-1-ol),10-O-[1-propyl-3-N-carbamoyl cholesteryl].

In an exemplary embodiment, the guide strand is 5′-fUfAfGfCfAfCfCfAfUfCfUfGfAfAfAfUfCfGfGfUmUmA-3′; and the passenger is 5′-mArCmUrGmArUmUrUmCrUmUrUmUrGmGrUmGrUmUrCdAchol-3′; wherein ‘f’ preceding a nucleoside=a 2′-deoxy-fluoro ribonucleoside (—F); ‘m’ preceding a nucleoside=a 2′-O-methyl ribonucleoside (—O-Me); ‘r’ preceding a nucleoside=a nucleoside with an unmodified ribose moiety (—OH); ‘d’ preceding a nucleoside=a 2′-deoxyribonucleoside (—H); chol indicates a cholesterol moiety.

An exemplary compound according to this embodiment is cholesterol-conjugated Teno18 (CWT-001), which is described in the Examples below.

In any of these embodiments, there may be a phosphate group at the 5′ position of the sugar of the nucleoside at the 5′ end of the guide strand (‘5′Phos’).

Thus in one embodiment, the guide strand is 5′-PhosfUfAfGfCfAfCfCfAfUfCfUfGfAfAfAfUfCfGfGfUmUmA-3′; and the passenger strand is 5′-mArCmUrGmArUmUrUmCrUmUrUmUrGmGrUmGrUmUrCdA-3′; wherein all internucleoside linkages of the guide strand and of the passenger strand are phosphodiester internucleoside linkages.

An exemplary compound according to this embodiment is Teno18, which is described in the Examples below.

In another embodiment, the guide strand is 5′-PhosfUfAfGfCfAfCfCfAfUfCfUfGfAfAfAfUfCfGfGfUmUmA-3′; and the passenger is 5′-mArCmUrGmArUmUrUmCrUmUrUmUrGmGrUmGrUmUrCdAchol-3′; wherein chol is a cholesterol moiety, for example a triethylene glycol cholesterol moiety; and wherein all internucleoside linkages of the guide strand and of the passenger strand are phosphodiester internucleoside linkages. An exemplary compound according to this embodiment is cholesterol-conjugated Teno18 (CWT-001), which is described in the Examples below.

The invention also provides a pharmaceutical composition comprising a miR-29 compound or salt thereof as described herein. The pharmaceutical composition typically comprises a pharmaceutically acceptable carrier or diluent. In certain embodiments, the pharmaceutically acceptable carrier or diluent is a sterile solution. The pharmaceutically acceptable carrier or diluent is a parenterally acceptable aqueous solution. For example, the pharmaceutically acceptable carrier or diluent may be phosphate buffered saline (PBS). The pharmaceutically acceptable carrier or diluent may comprise additional substances such as EDTA, for example at a final concentration of 0.001%-0.005%, e.g., 0.003% (100 μM). In certain embodiments, the pharmaceutical composition does not include non-liquid carriers, e.g., the miR-29 compound or salt thereof is not comprised in, or administered in, to or with solid carriers such as implantable scaffolds.

The invention further provides a container comprising a miR-29 compound or salt thereof as described herein. The miR-29 compound or salt thereof is typically provided in a therapeutically effective amount. A therapeutically effective amount is an amount of the miR-29 compound or salt thereof that when administered to a subject reduces or inhibits collagen 3 and typically results in an improvement in disease symptoms, e.g., reduced pain or disability, improved functionality, and/or slower disease progression. The container typically comprises a label affixed to the container. The container may be a glass vial (e.g., a single use glass vial) containing the pharmaceutical composition described herein. The container may be a syringe or an injector.

The invention also provides a miR-29 compound or salt thereof, or a pharmaceutical composition as described herein, for use in therapy.

Accordingly, also provided herein is the use of a miR-29 compound or salt thereof, or the use of a pharmaceutical composition as described herein, for the manufacture of a medicament.

Also provided is a method of treating a disease or condition comprising administering a therapeutically effective amount of a miR-29 compound or salt thereof, or a pharmaceutical composition as described herein, to a subject in need thereof.

The terms “therapy”, “medicament”, “treating” and “treatment” and the like include curative as well as prophylactic methods.

Thus in a preferred embodiment, provided herein is a miR-29 compound or a salt thereof for use in therapy, for example comprising intralesional administration (e.g., a single intralesional administration) by injection of the miR-29 compound or salt thereof, wherein the miR-29 compound or salt thereof comprises the guide strand 5′-fUfAfGfCfAfCfCfAfUfCfUfGfAfAfAfUfCfGfGfUmUmA-3′ and the passenger 5′-mArCmUrGmArUmUrUmCrUmUrUmUrGmGrUmGrUmUrCdA-3′, optionally wherein a lipophilic (e.g., cholesterol) moiety is covalently attached to the 3′ end of the passenger strand.

In certain embodiments, the treatment is for collagen dysregulation, in particular collagen overproduction, for example collagen 3 overproduction. The term “overproduction” refers to increased collagen production relative to corresponding healthy tissue. The overproduction is typically locally. For example, tendon damage such as tendinopathy involves local collagen 3 overproduction. Fibrotic conditions such as Peyronie's disease or Dupuytren's disease also involve local collagen 3 overproduction. In one embodiment therefore the miR-29 compound or salt thereof, or the pharmaceutical composition described herein, is for use in treating tendon damage such as tendinopathy, or tissue fibrosis such as Peyronie's disease or Dupuytren's disease. In one embodiment, the miR-29 compound or salt thereof, or the pharmaceutical composition described herein, is for use in treating tendon damage. The tendon damage may be tendinopathy. The tendon damage may be chronic tendinopathy. The tendon damage may be lateral epicondylitis.

The subject is typically a human. However, another preferred subject is a horse, particularly a horse that has been bred for equine sport.

In another aspect, the invention provides dosages and treatment regimens for miR-29 compounds, salts thereof, and pharmaceutical compositions comprising the same. The dosages and treatment regimens provided herein are suitable for a range of miR-29 compounds, i.e., the applicability of the disclosure of dosages and treatment regimens herein includes but is not limited to the novel miR-29 compounds provided herein. Nevertheless, the dosages and treatment regimens preferably involve the novel miR-29 compounds, because the inventors have found that the dosages and treatment regimens are particularly suitable for these compounds.

The miR-29 compound for use with the dosage and treatment regimens disclosed herein can be chemically modified, as described above. This means that at least one sugar, internucleoside linkage or nucleobase has a chemical structure that differs from that in naturally-occurring miR-29, again as described above. In certain embodiments, the miR-29 compound or salt thereof comprises modified sugars but does not comprise modified internucleoside linkages or modified nucleobases (the miR-29 compound or salt thereof does not comprise modified internucleoside linkages and it does not comprise modified nucleobases). For example, all of the internucleoside linkages of the guide strand and/or the passenger strand can be phosphodiester internucleoside linkages. The miR-29 compounds may also be conjugated (i.e., covalently linked) to a lipophilic moiety such as a cholesterol moiety, typically at the 3′ end of the passenger strand. The miR-29 compound or salt thereof may be any one of the specific compounds or salts described herein, or it can be a different miR-29 compound or salt thereof.

In one embodiment of this aspect, the invention therefore provides a miR-29 compound or a salt thereof for use in therapy comprising a single intralesional administration by injection of a dose of the miR-29 compound or the salt thereof. The corresponding use for the manufacture of a medicament, and the corresponding method of treatment, are also provided, as described above and below.

In certain embodiments, treatment comprises no more than two, or no more than three, intralesional administrations by injection of the miR-29 compound or the salt thereof. In other embodiment, the treatment comprises a single intralesional administration by injection of the miR-29 compound or the salt thereof.

The treatment may be for tendon damage (e.g., tendinopathy) and the administration is into the damaged area of the tendon (intra-tendon, or intratendinous).

The treatment may be for Peyronie's disease and the administration is into the affected area of the tunica albuginea of the subject.

The treatment may be for Dupuytren's disease and the administration is into the affected area of the palmar fascia of the subject.

In any of these embodiments, a single dose of the miR-29 compound may be between 10 μg and 5000 μg of the miR-29 compound, or the equivalent dose of the salt thereof. As explained in further detail below, unless otherwise indicated the dose refers to the weight of the oligonucleotide portion of the guide and passenger strand (i.e., excluding any conjugate groups such as a cholesterol moiety), as a free acid.

A single dose may therefore be between 10 μg and 4500 μg, 10 μg and 4000 μg, 10 μg and 3500 μg, 10 μg and 3000 μg, or between 10 μg and 2500 μg of the miR-29 compound, or the equivalent dose of the salt thereof.

A single dose may be between 47 μg and 4740 μg of the miR-29 compound, or the equivalent dose of the salt thereof. A single dose may be between 47 μg and 4270 μg of the miR-29 compound, or the equivalent dose of the salt thereof. A single dose may be between 47 μg and 1900 μg of the miR-29 compound, or the equivalent dose of the salt thereof. A single dose may be between 47 μg and 1420 μg of the miR-29 compound, or the equivalent dose of the salt thereof. A single dose may be between 47 μg and 475 μg of the miR-29 compound, or the equivalent dose of the salt thereof.

A single dose may be between 95 μg and 4740 μg of the miR-29 compound, or the equivalent dose of the salt thereof. A single dose may be between 95 μg and 4270 μg of the miR-29 compound, or the equivalent dose of the salt thereof. A single dose may be between 95 μg and 1900 μg of the miR-29 compound, or the equivalent dose of the salt thereof. A single dose may be between 95 μg and 1420 μg of the miR-29 compound, or the equivalent dose of the salt thereof. A single dose may be between 95 μg and 475 μg of the miR-29 compound, or the equivalent dose of the salt thereof.

A single dose may be between 190 μg and 4740 μg of the miR-29 compound, or the equivalent dose of the salt thereof. A single dose may be between 190 μg and 4270 μg of the miR-29 compound, or the equivalent dose of the salt thereof. A single dose may be between 190 μg and 1900 μg of the miR-29 compound, or the equivalent dose of the salt thereof. A single dose may be between 190 μg and 1420 μg of the miR-29 compound, or the equivalent dose of the salt thereof. A single dose may be between 190 μg and 475 μg of the miR-29 compound, or the equivalent dose of the salt thereof.

A single dose may be between 475 μg and 4740 μg of the miR-29 compound, or the equivalent dose of the salt thereof. A single dose may be between 475 μg and 4270 μg of the miR-29 compound, or the equivalent dose of the salt thereof. A single dose may be between 475 μg and 1900 μg of the miR-29 compound, or the equivalent dose of the salt thereof. A single dose may be between 475 μg and 1420 μg of the miR-29 compound, or the equivalent dose of the salt thereof.

A single dose may be between 1420 μg and 4740 μg of the miR-29 compound, or the equivalent dose of the salt thereof. A single dose may be between 1420 μg and 4270 μg of the miR-29 compound, or the equivalent dose of the salt thereof. A single dose may be between 1420 μg and 1900 μg of the miR-29 compound, or the equivalent dose of the salt thereof.

In certain embodiments, a single dose is between 190 μg and 1420 μg of the miR-29 compound, or the equivalent dose of the salt thereof. A single dose may be about 190 μg, 475 μg, 1420 μg or 4270 μg of the miR-29 compound, or the equivalent dose of the salt thereof. For example, a single dose may be about 190 μg, 475 μg or 1420 μg of the miR-29 compound, or the equivalent dose of the salt thereof. This means that a single dose may be about 190 μg of the miR-29 compound, or the equivalent dose of the salt thereof. A single dose may be about 475 μg of the miR-29 compound, or the equivalent dose of the salt thereof. A single dose may be about or 1420 μg of the miR-29 compound, or the equivalent dose of the salt thereof.

In a preferred embodiment, a single dose is between about 190 μg and about 475 μg of the miR-29 compound, or the equivalent dose of the salt thereof. For example, a single dose may be about 190 μg of the miR-29 compound, or the equivalent dose of the salt thereof. A single dose may also be about 475 μg of the miR-29 compound, or the equivalent dose of the salt thereof. In a preferred embodiment, a single dose is about 190 μg of the miR-29 compound, or the equivalent dose of the salt thereof.

In one embodiment therefore, the invention provides a miR-29 compound or a salt thereof for use in the treatment of tendon damage in a subject, wherein the treatment comprises a single administration by injection of a dose of at least 47 μg of the miR-29 compound, or the equivalent dose of the salt thereof, into the damaged tendon of the subject.

In another embodiment, the invention provides a miR-29 compound or a salt thereof for use in the treatment of Peyronie's disease in a subject, wherein the treatment comprises a single administration by injection of a dose of at least 47 μg of the miR-29 compound, or the equivalent dose of the salt thereof, into the affected area of the tunica albuginea of the subject.

In a further embodiment, the invention provides a miR-29 compound or a salt thereof for use in the treatment of Dupuytren's disease in a subject, wherein the treatment comprises a single administration by injection of a dose of at least 47 μg of the miR-29 compound, or the equivalent dose of the salt thereof, into the affected area of the palmar fascia of the subject.

In certain embodiments, the single dose of the miR-29 compound is at least 95 μg, at least 190 μg, at least 475 μg, at least 1420 μg, at least 1900 μg, at least 4270 μg, or the equivalent dose of the salt thereof.

For example, the single dose of the miR-29 compound may be at least 95 μg (or at least 190 μg, at least 475 μg, at least 1420 μg, at least 1900 μg, at least 4270 μg) and no more than 4740 μg of the miR-29 compound, or the equivalent dose of the salt thereof. Thus a single dose may be within a certain range as described above.

More generally, a single dose of the miR-29 compound may be at least or about, 10 μg, 15 μg, 20 μg, 25 μg, 30 μg, 35 μg, 40 μg, 45 μg, 47 μg, 50 μg, 55 μg, 60 μg, 75 μg, 80 μg, 95 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 350 μg, 360 μg, 380 μg, 400 μg, 420 μg, 440 μg, 460 μg, 470 μg, 475 μg, 480 μg, 500 μg, 550 μg, 600 μg, 650 μg, 700 μg, 750 μg, 800 μg, 850 μg, 900 μg, 950 μg, 1000 μg, 1100 μg, 1200 μg, 1300 μg, 1400 μg, 1420 μg, 1500 μg, 1600 μg, 1700 μg, 1800 μg, 1900 μg or 2000 μg of the miR-29 compound, or the equivalent dose of the salt thereof. In certain embodiments a higher dose may be used, such as between 4000 μg and 4500 μg, e.g., 4270 μg.

In certain embodiments, which can be combined with other embodiments described herein, a single dose of the miR-29 compound is no more than 190 μg, 200 μg, 220 μg, 240 μg, 260 μg, 280 μg, 300 μg, 320 μg, 340 μg, 350 μg, 360 μg, 380 μg, 400 μg, 420 μg, 440 μg, 460 μg, 470 μg, 475 μg, 480 μg, 500 μg, 550 μg, 600 μg, 650 μg, 700 μg, 750 μg, 800 μg, 850 μg, 900 μg, 950 μg, 1000 μg, 1100 μg, 1200 μg, 1300 μg, 1400 μg, 1420 μg, 1500 μg, 1600 μg, 1700 μg, 1800 μg, 1900 μg, 2000 μg, 2200 μg, 2400 μg, 2600 μg, 2800 μg, 3000 μg, 3200 μg, 3400 μg, 3600 μg, 3800 μg, 4000 μg, 4200 μg, 4270 μg, 4400 μg, 4600 μg, 4740 μg, 4800 μg or 5000 μg of the miR-29 compound, or the equivalent dose of the salt thereof. In a specific embodiment, a single dose of the miR-29 compound is no more than 190 μg, or the equivalent dose of the salt thereof. In a specific embodiments a single dose of the miR-29 compound is no more than 475 μg, or the equivalent dose of the salt thereof. In a specific embodiment, a single dose of the miR-29 compound is no more than 1420 μg, or the equivalent dose of the salt thereof. In a specific embodiment, a single dose of the miR-29 compound is no more than 1900 μg, or the equivalent dose of the salt thereof. In a specific embodiment, a single dose of the miR-29 compound is no more than 4270 μg, or the equivalent dose of the salt thereof.

Where tendon damage is treated, the tendon damage may be tendinopathy. The tendon damage may be chronic tendinopathy. The tendon damage may be lateral epicondylitis, medial epicondylitis, rotator cuff tendinopathy, common extensor origin tendinopathy, common flexor origin tendinopathy, gluteal tendinopathy (greater trochanteric pain syndrome, GTPS), patellar tendinopathy, jumper's knee, plantar fasciitis, Achilles tendinopathy, peroneal tendinopathy, supraspinatus syndrome, or a combination thereof. In one embodiment, the tendon damage is upper limb tendinopathy (including lateral epicondylitis, golfer's elbow, rotator cuff tendinopathy and De Quervain's disease). In a particular embodiment, the tendon damage is lateral epicondylitis.

The subject is typically a human. However, another preferred subject is a horse, particularly a horse that has been bred for equine sport.

In certain embodiments, the miR-29 compound or salt thereof is administered in a volume of 0.5 ml-5 ml, e.g., 0.5 ml-1.5 ml, such as 0.8 ml-1.2 ml, for example 0.9 ml-1.1 ml. For example, the volume may be about 0.5 ml, 0.75 ml, 1 ml, 1.25 ml, 1.5 ml, 1.75 ml, 2 ml, 2.5 ml, 3 ml, 3.5 ml, 4 ml, 4.5 ml or about 5 ml. In a specific embodiment, the volume is about 1 ml. For example, in one embodiment the miR-29 compound or salt thereof is administered in a volume of about 1 ml of a sterile aqueous solution.

The miR-29 compound or salt thereof may be administered at a concentration that corresponds to any of the dosages indicated above. For example, where the dosage is 190 μg of the miR-29 compound, the compound may be administered at a concentration of 190 μg/ml.

In certain embodiments, the miR-29 compound or salt thereof is any one of the specific miR-29 compounds of salts thereof as provided herein.

In a specific embodiment, the invention therefore provides a miR-29 compound or a salt thereof for use in therapy comprising a single intralesional administration by injection of a dose of the miR-29 compound or the salt thereof, wherein the guide strand is 5′-fUfAfGfCfAfCfCfAfUfCfUfGfAfAfAfUfCfGfGfUmUmA-3′, the passenger strand is 5′-mArCmUrGmArUmUrUmCrUmUrUmUrGmGrUmGrUmUrCdA-3′ and comprises a cholesterol moiety covalently attached to the 3′ end of the passenger strand (5′-mArCmUrGmArUmUrUmCrUmUrUmUrGmGrUmGrUmUrCdAchol-3′), and the dose of the miR-29 compound (including the weight of the cholesterol moiety) is between 50 μg and 5000 μg, for example between 200 μg and 4500 μg, such as between 200 μg and 1500 μg, between 200 μg and 500 μg, between 500 μg and 1500 μg, or between 1500 μg and 4500 μg (e.g., about 100 μg, 200 μg, 250 μg, 300 μg, 500 μg, 1000 μg, 1500 μg or about 4500 μg), or the equivalent dose of the salt thereof; wherein ‘f’ preceding a nucleoside=a 2′-deoxy-fluoro ribonucleoside (—F); ‘m’ preceding a nucleoside=a 2′-O-methyl ribonucleoside (—O-Me); ‘r’ preceding a nucleoside=a nucleoside with an unmodified ribose moiety (—OH); ‘d’ preceding a nucleoside=a 2′-deoxyribonucleoside (—H). In a preferred embodiment, the dose of the miR-29 compound (including the weight of the cholesterol moiety) is between about 200 μg and about 500 μg, or the equivalent dose of the salt thereof. For example, the dose of the miR-29 compound (including the weight of the cholesterol moiety) may be about 200 μg, or the equivalent dose of the salt thereof. The dose of the miR-29 compound (including the weight of the cholesterol moiety) may also be about 500 μg, or the equivalent dose of the salt thereof. In a specific embodiment, the dose of the miR-29 compound (including the weight of the cholesterol moiety) is about 200 μg, or the equivalent dose of the salt thereof.

In one embodiment therefore the guide strand of the miR-29 compound or salt thereof is 5′-fUfAfGfCfAfCfCfAfUfCfUfGfAfAfAfUfCfGfGfUmUmA-3′, the passenger strand of the miR-29 compound or salt thereof is 5′-mArCmUrGmArUmUrUmCrUmUrUmUrGmGrUmGrUmUrCdA-3′ and comprises a cholesterol moiety covalently attached to the 3′ end of the passenger strand, and the dose of the miR-29 compound including the weight of the cholesterol moiety is between 50 μg and 5000 μg, for example 200 μg, 500 μg, 1500 μg or 4500 μg, or the equivalent dose of the salt thereof.

This means that the dose of the miR-29 compound including the weight of the cholesterol moiety may be about 200 μg, or the equivalent dose of the salt thereof. The dose of the miR-29 compound including the weight of the cholesterol moiety may be about 500 μg, or the equivalent dose of the salt thereof. The dose of the miR-29 compound including the weight of the cholesterol moiety may be about 1500 μg, or the equivalent dose of the salt thereof. The dose of the miR-29 compound including the weight of the cholesterol moiety may be about 4500 μg, or the equivalent dose of the salt thereof. In a preferred embodiment, the dose of the miR-29 compound (including the weight of the cholesterol moiety) is between about 200 μg and about 500 μg, or the equivalent dose of the salt thereof. For example, the dose of the miR-29 compound (including the weight of the cholesterol moiety) may be about 200 μg, or the equivalent dose of the salt thereof. The dose of the miR-29 compound (including the weight of the cholesterol moiety) may also be about 500 μg, or the equivalent dose of the salt thereof. In a specific embodiment, the dose of the miR-29 compound (including the weight of the cholesterol moiety) is about 200 μg, or the equivalent dose of the salt thereof. The therapy may be for tissue fibrosis, such as Peyronie's disease, or Dupuytren's disease. The therapy may be for tendon damage. The tendon damage may be tendinopathy. The tendon damage may be chronic tendinopathy. The tendon damage may be lateral epicondylitis. The miR-29 compound may be administered as a salt, for example as a sodium salt. The carrier may be a sterile aqueous solution that is parenterally acceptable, for example PBS.

In another aspect, the invention provides a process for making a miR-29 compound in accordance with the invention. The invention also provides a process for making a pharmaceutical composition, comprising providing a miR-29 compound in accordance with the invention and combining the compound with a pharmaceutically acceptable carrier or diluent.

### MicroRNA-29 Compounds

A “microRNA-29” compound as used herein refers to an oligonucleotide that has the biological function of a natural endogenous miR-29, which includes miR-29a, miR-29b (which includes miR-29b-1 and miR-29b-2) and miR-29c. That function includes the targeting of mRNA by complementarily binding (hybridising) to the 3′ untranslated region (3′UTR) of any target mRNA that contains one or more binding sites for miR-29 (e.g., one or more binding sites for miR-29a), thereby causing its degradation and/or translational repression. Target mRNAs that harbour binding sites for a miR-29 typically encode certain extracellular matrix proteins, such as collagen, for example collagen 3 (col3a1). The miR-29 compounds described herein mimic the biological function of a natural miR-29, but may be modified relative to a natural miR-29 (e.g., natural miR-29a) so as to, e.g., enhance its activity, stability and/or cellular uptake, but without causing immunogenicity. Modifications may include nucleobase sequence modifications, and/or chemical modifications at one or more sugar moieties, internucleoside linkages, and/or nucleobases. Modifications also include conjugation, e.g., to a lipophilic moiety. In one embodiment, the compound has the biological function of human mature miR-29a, i.e., it is a mimic of human mature miR-29a.

The miR-29 compounds described herein are capable of reducing or inhibiting the expression of a protein that is encoded by an mRNA that harbours one or more binding sites for miR-29, e.g., one or more binding sites for miR-29a. For example, the miR-29 compounds described herein are capable of reducing or inhibiting collagen expression, for example collagen 3 (col3a1) expression. The miR-29 compounds may reduce collagen expression to a greater extent than native miR-29a. For example, the miR-29 compounds may reduce collagen expression by at least 10%, at least 20%, or at least 30% or more compared to native miR-29a. Suitable assays to assess reduction in collagen (col3a1) expression are known to the person skilled in the art, such as the luciferase assay or the qPCR assay described in Example 1 below. Protein levels may also be assessed by methods well known in the art (e.g., Western Blot).

The miR-29 compounds described herein are capable of cellular uptake, for example they can be taken up in vivo by target cells (such as tenocytes, or fibroblasts) without the use of transfection agents or other transfection vehicles such as liposomes. Conjugation of the miR-29 compound with a lipophilic moiety such as a cholesterol moiety enhances cellular uptake. Capability of cellular uptake can be assessed by methods known to the skilled person in the art, for example measuring miR-29 activity in the in vitro collagen 3 luciferase reporter assay.

The miR-29 compounds described herein are stable. A stable miR-29 compound substantially retains activity in the in vitro collagen 3 luciferase reporter assay after storage at 37° C. for three months.

The miR-29 compounds described herein are stable also in the sense that they have a longer in vivo half-life as a result of 2′ sugar modifications, compared to unmodified (natural) miR-29.

Double-stranded RNA can stimulate innate immune responses in mammals. For example, it has been described that miR-29a can bind to receptors of the Toll-like receptor (TLR) family in immune cells and trigger an inflammatory response (ref. 5). Immune stimulation could significantly limit the in vivo application of miRNA therapy. Compounds that can effectively mediate miRNA activity but avoid or minimize immune activation are therefore desirable. The miR-29 compounds provided herein are non-immunogenic when administered to a subject, e.g., a human subject. Suitable assays are known to the person skilled in the art to assess non-immunogenicity, such as the TNF-alpha assay using human macrophages as described in Example 1 below. Preferred compounds do not induce the expression of TNF-alpha, or they induce the expression of no more than 10 pg/ml TNF-alpha, as measured in the supernatants of cultured primary human macrophages 24 hours after treatment with the compound.

As the compounds described herein do not induce an undesirable immune response, the compounds do not need to be protected from recognition by the immune system by encapsulation into liposomes or similar means. The compounds described herein are therefore effective in vivo even when administered in a simple aqueous carrier, e.g., PBS. This significantly simplifies preparation and administration of the therapy and avoids the introduction of further foreign components into the subject which could raise safety concerns. The compounds described herein therefore have the advantage of effectively mimicking the function of natural miR-29 (e.g., miR-29a) but having a higher activity and stability yet being non-immunogenic. The Examples also show that the compounds can be anti-inflammatory.

The exemplary miR-29 compounds described in the Examples are therefore chemically synthesised mimics of miR-29 which have improved stability, activity and cellular uptake, while being non-immunogenic. The nucleobase sequence of the compounds is closest to that of miR-29a, however as noted above functional mimicry can extend to other miR-29 family members. The miR-29 compounds are stable in an aqueous carrier and remain active even after storage at 37° C. for three months. To increase stability and activity, certain miR-29 compounds have been chemically modified via the introduction of 2′-fluoro and 2′-O-methyl groups in the guide strand, and 2′-O-methyl groups in the passenger strand. The passenger strand may additionally contain a 3′ cholesterol group to increase cellular uptake.

Administration of the compounds to sites of collagen dysregulation, e.g., damaged tendon or fibrotic tissue, restores miR-29a function. The miR-29 compounds described herein have a well-defined mode of action. The compounds directly target the key pathways in collagen production associated with a range of pathogenic conditions, including tendon damage such as tendinopathy and other diseases that require a reduction or inhibition of collagen (in particular collagen 3) production, e.g., tissue fibrosis. Moreover, treatment with the miR-29 compounds described herein does not require invasive procedures and can be delivered at the point of initial diagnosis, thereby initiating recovery at the very earliest time.

As described in the Examples, in vitro and in vivo assays have demonstrated that miR-29 compounds described herein are able to significantly reduce the expression of collagen 3, for example in tenocytes and fibroblasts. The Examples also show that a single intra-tendon injection of miR-29 compound significantly reduced collagen 3 expression and improved tendon structure in the mouse tendon injury models.

In terms of safety, studies in rat and dog monitored a variety of features including: clinical observations, body weight, haematology, coagulation or clinical chemistry parameters and gross necropsy findings acutely and after a 14 day follow-up. These studies were also designed to show safety pharmacology effects and no changes were seen in the Irwin behavioural profile in rats nor any cardiovascular effects in telemeterised dogs. Respiratory parameters in the rats remained unaffected. The Examples also show that following local (intra-lesional) administration, negligible distribution in other tissues is observed and any systemic miR29a is rapidly degraded. Both in vitro and in vivo data in isolated human macrophages and the mouse tendon injury model have provided evidence that miR-29 compounds described herein are non-immunogenic. Furthermore, treatment with miR-29 compounds described herein did not show any off-target effects.

The inventors found that double-stranded miR-29 compounds (comprising a guide and a passenger strand) are more active than single-stranded miR-29 compounds (comprising a guide strand only; e.g., Teno36 in Example 1). The miR-29 compounds described herein are therefore typically double-stranded. The two strands are substantially complementary to each other such that they form a duplex.

### Length

The guide strand may be between 20 and 24 linked nucleosides in length. For example, the guide strand may be 20, 21, 22, 23 or 24 linked nucleosides in length. In one embodiment, the guide strand is between 20 and 23 linked nucleosides in length. In one embodiment, the guide strand is between 20 and 22 linked nucleosides in length. In one embodiment, the guide strand is between 21 and 23 linked nucleosides in length. In one embodiment, the guide strand is 21 or 22 linked nucleosides in length. In one embodiment, the guide strand is 22 or 23 linked nucleosides in length. In a preferred embodiment, the guide strand is 22 linked nucleosides in length.

The passenger strand may be between 19 and 23, or between 20 and 24 linked nucleosides in length. For example, the passenger strand may be 19, 20, 21, 22, 23 or 24 linked nucleosides in length. In one embodiment, the passenger strand is between 19 and 22 linked nucleosides in length. In one embodiment, the passenger strand is between 19 and 21 linked nucleosides in length. In one embodiment, the passenger strand is 20 or 21 linked nucleosides in length. In one embodiment, the passenger strand is 21 or 22 linked nucleosides in length. In a preferred embodiment, the passenger strand is 21 linked nucleosides in length.

### Overhang

The guide and passenger strands of the miR-29 compound form a duplex due to portions of sequence complementarity between the respective oligonucleotides of the guide and passenger strand. The guide and passenger strand may form a duplex such that the guide strand has a 3′ overhang, e.g., of two nucleosides, relative to the passenger strand, and the passenger strand has a 3′ overhang, e.g., of one nucleoside, relative to the guide strand. The guide and passenger strands may form a duplex such that the guide strand has a 3′ overhang, e.g., of two nucleosides, relative to the passenger strand.

### Mismatches

There may be mismatches between corresponding nucleobases of the guide strand and the passenger strand when aligned, i.e., when in a duplex. A mismatch exists where the corresponding nucleobases are not complementary. Complementary nucleobases are U and A, and C and G. For example, there may be 3 to 8 mismatches, for example 3, 4, 5, 6, 7 or 8 mismatches. In one embodiment, there are 3 to 6 mismatches. In one embodiment, there are 5 or 6 mismatches. In one embodiment, there are 6 mismatches. For example, the mismatches may be at positions 3, 10 to 12, 18 and 20 (numbering relative to the passenger strand). The inventors have surprisingly found that miR-29 compounds that contain mismatches between the guide and the passenger strand have a higher activity (e.g., as determined in the collagen 3 luciferase assay) compared to miR-29 compounds that have fewer or no mismatches.

### Conjugates

The miR-29 compounds described herein may comprise one or more conjugate groups covalently attached to the oligonucleotide(s) of the compound. An exemplary conjugate group is a cholesterol moiety, although other conjugate moieties that improve the pharmacological properties (e.g., the cellular uptake) of the oligonucleotide are known in the art and can be used instead of a cholesterol moiety. The conjugate groups are therefore typically lipophilic moieties.

In one embodiment, the miR-29 compound comprises a cholesterol moiety covalently attached to one or more oligonucleotide strands of the miR-29 compound. In one embodiment, the cholesterol moiety is covalently attached to the passenger strand. In one embodiment, the cholesterol moiety is covalently attached to the passenger strand, and the guide strand is unconjugated. In one embodiment, the cholesterol moiety is covalently attached to the 3′ end of the passenger strand. In any of these embodiments, the cholesterol moiety may be attached via a linker, e.g., a triethyleneglycol. Thus in one embodiment the cholesterol moiety is a triethyleneglycol cholesterol moiety. An exemplary cholesterol moiety is 3′phosphopropy-2-ol,2,2′-[Ethane-1,2-diylbis(oxy)]di(ethan-1-ol), 10-O-[1-propyl-3-N-carbamoylcholesteryl].

### Salts

The miR-29 compounds described herein can be provided as a salt. The term “compound” therefore includes a salt of the compound. The salt is a pharmaceutically acceptable salt, i.e., the salt retains the biological activity of the compound and does not impart toxicological effects thereto. Examples of pharmaceutically acceptable salts include sodium salts and potassium salt. In one embodiment, the miR-29 compound is a sodium salt.

### CWT-001

A particular miR-29 compound described in the Examples is CWT-001. CWT-001 is a double-stranded chemically modified mimic of at least miR-29a, comprising a guide (antisense) and a passenger (sense) strand at a 1:1 ratio. The nucleobase sequences and chemical modifications of the guide and passenger strands correspond to those of ‘Teno18’ (T18).

### CWT-001 Guide Strand

CWT-001 inhibits the expression of at least the same target genes as naturally-occurring miR-29a, including collagen 3. The sugar residues have been chemically modified via the introduction of 2′-fluoro and 2′-O-methyl groups, which increase stability and activity of the compound. The guide strand of CWT-001 has the same nucleobase sequence as the naturally-occurring guide strand of human mature miR-29a (“hsa-miR-29a-3p”), which is:

(SEQ ID NO: 1)
5′-UAGCACCAUCUGAAAUCGGUUA-3′

Each nucleoside is a 2′-substituted nucleoside, which means that the nucleoside comprises a sugar moiety that comprises at least one 2′-substituent group other than H or OH. The guide strand of CWT-001 has the following formula:

5′-fUfAfGfCfAfCfCfAfUfCfUfGfAfAfAfUfCfGfGfUmUmA-3′

wherein “f” denotes a nucleoside containing a fluorine at the 2′ position of the ribose ring, and “m” denotes a nucleoside containing an O-methyl group at the 2′ position of the ribose ring.

The internucleoside linkages are phosphodiester internucleoside linkages throughout.

The chemical name of the CWT-001 guide strand is:
RNA (5′Phospho (2′deoxy-2′Fluoro)U-(2′deoxy-2′Fluoro)A-(2′deoxy-2′Fluoro)G-(2′deoxy-2′Fluoro)C-(2′deoxy-2′Fluoro)A-(2′deoxy-2′Fluoro)C-(2′deoxy-2′Fluoro)C-(2′deoxy-2′Fluoro)A-(2′deoxy-2′Fluoro)U-(2′deoxy-2′Fluoro)C-(2′deoxy-2′Fluoro)U-(2′deoxy-2′Fluoro)U-(2′deoxy-2′Fluoro)G-(2′deoxy-2′Fluoro)A-(2′deoxy-2′Fluoro)A-(2′deoxy-2′Fluoro)A-(2′deoxy-2′Fluoro)U-(2′deoxy-2′Fluoro)C-(2′deoxy-2′Fluoro)G-(2′deoxy-2′Fluoro)G-(2′deoxy-2′Fluoro)U-(2′deoxy-2′OMethyl)U-(2′OMethyl)A), optionally as a salt, e.g., a sodium salt.

The structural formula of the CWT-001 guide strand as a free acid is shown below:

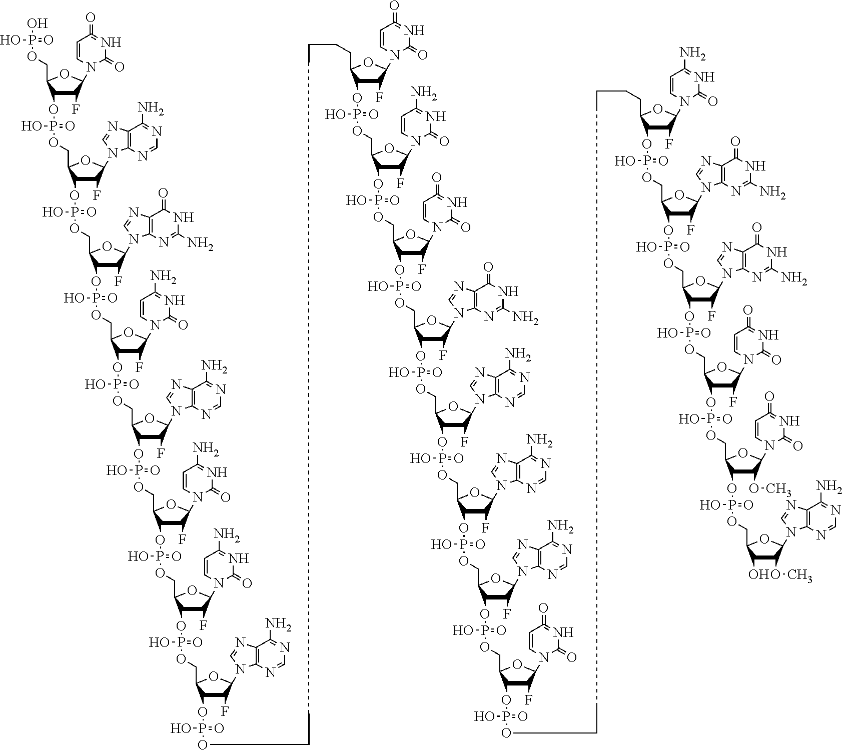

The structural formula of the CWT-001 guide strand in ionised form (i.e., the anion of a salt) is shown below:

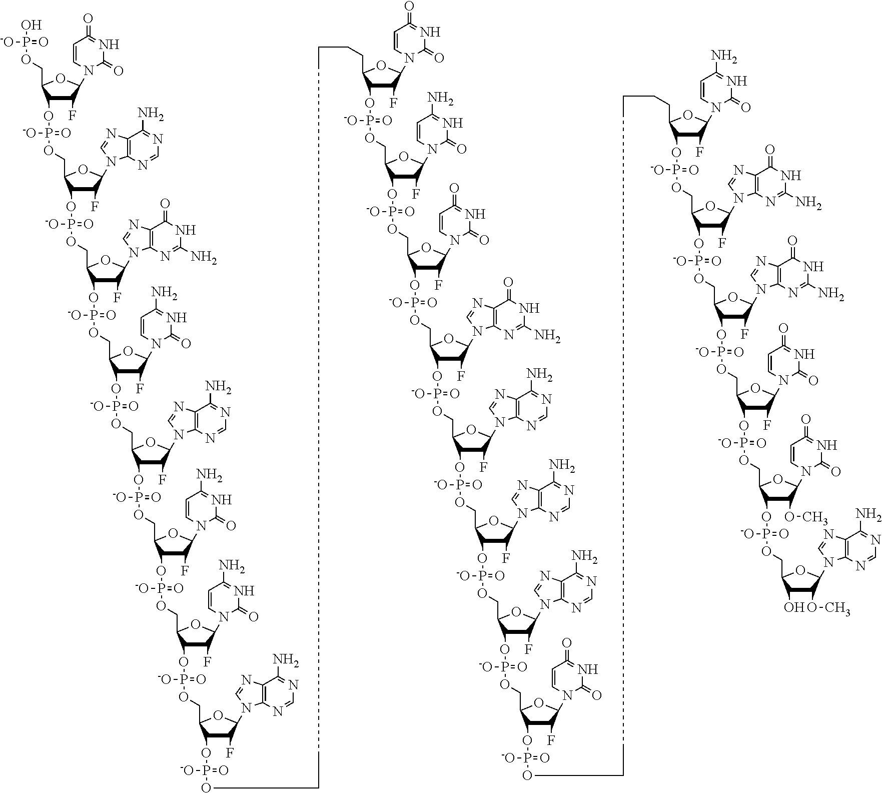

The structural formula of the CWT-001 guide strand, as a sodium salt, is shown below and in FIG. 1A:

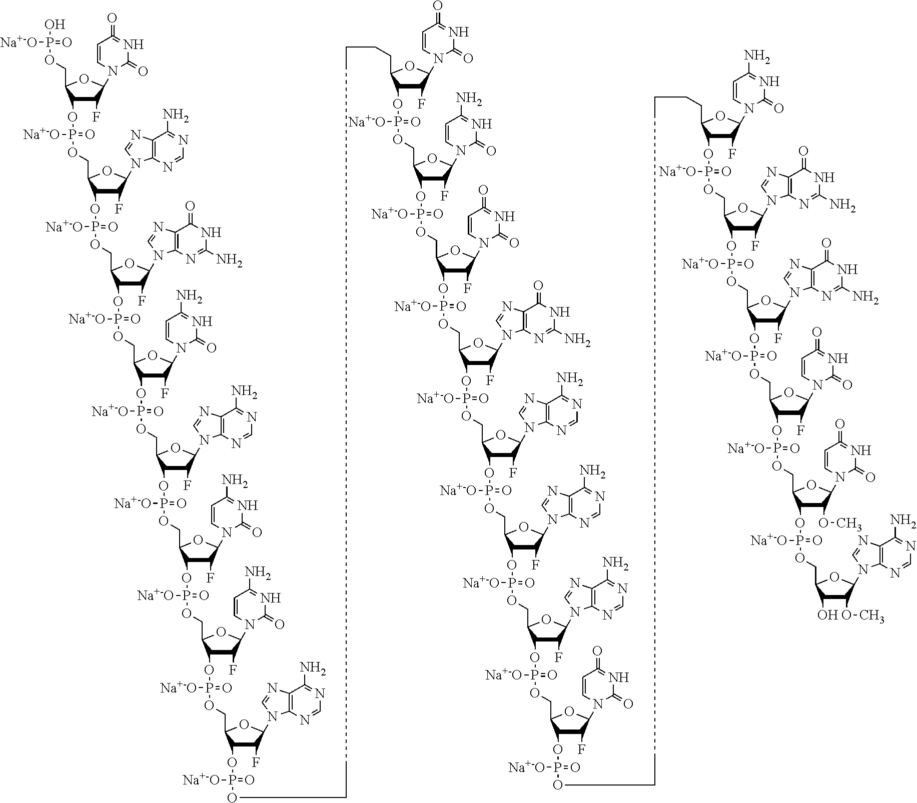

### CWT-001 Passenger Strand

The passenger strand of CWT-001 has the same nucleobase sequence as the naturally-occurring passenger strand of human mature miR-29a (“hsa-miR-29a-5p”) except that the G at the 3′ end is not present in CWT-001. The nucleobase sequence of the CWT-001 passenger strand therefore is:

(SEQ ID NO: 3)
5′-ACUGAUUUCUUUUGGUGUUCA-3′.

The passenger strand contains nucleosides comprising a modified sugar moiety (2′-O-methyl), nucleosides comprising an unmodified sugar moiety, and a nucleoside comprising a deoxy ribose sugar moiety. The passenger strand of CWT-001 has the following formula:

5′-mArCmUrGmArUmUrUmCrUmUrUmUrGmGrUmGrUmUrCdA-3′

wherein “m” denotes a nucleoside containing an O-methyl group at the 2′ position of the ribose ring, “r” denotes a ribonucleoside, i.e., containing an OH group at the 2′ position of the ribose ring, “d” denotes a deoxy ribose nucleoside.

The internucleoside linkages are phosphodiester internucleoside linkages throughout.

The passenger strand of CWT-001 comprises a cholesterol moiety, e.g., a triethyleneglycol cholesterol moiety, covalently attached to the 3′ end of the passenger strand. The cholesterol moiety improves the pharmacological properties of the miR-29a compound, e.g., the cholesterol moiety increases the cellular uptake of the oligonucleotide.

The chemical name of the CWT-001 passenger strand, as a sodium salt, is:
RNA ((2′OMethyl)A-C-((2′OMethyl)U-G-(2′OMethyl)A-U-(2′OMethyl)U-U-(2′OMethyl)C-U-(2′OMethyl)U-U-(2′OMethyl)U-G-(2′OMethyl)G-U-(2′OMethyl)G-U-(2′OMethyl)U-C-(2′Deoxy)A-3′phosphopropy-2-ol,2,2′-[Ethane-1,2-diylbis(oxy)]di(ethan-1-ol), 10-O-[1-propyl-3-N-carbamoylcholesteryl], optionally as a salt, e.g., a sodium salt.

Thus the passenger strand comprising a cholesterol moiety (e.g., a triethyleneglycol cholesterol moiety) covalently attached to the 3′ end of the passenger strand can also be denoted by the following formula:

5′-mArCmUrGmArUmUrUmCrUmUrUmUrGmGrUmGrUmUrCdA
chol-3.

The structural formula of the CWT-001 passenger strand as a free acid is shown below:

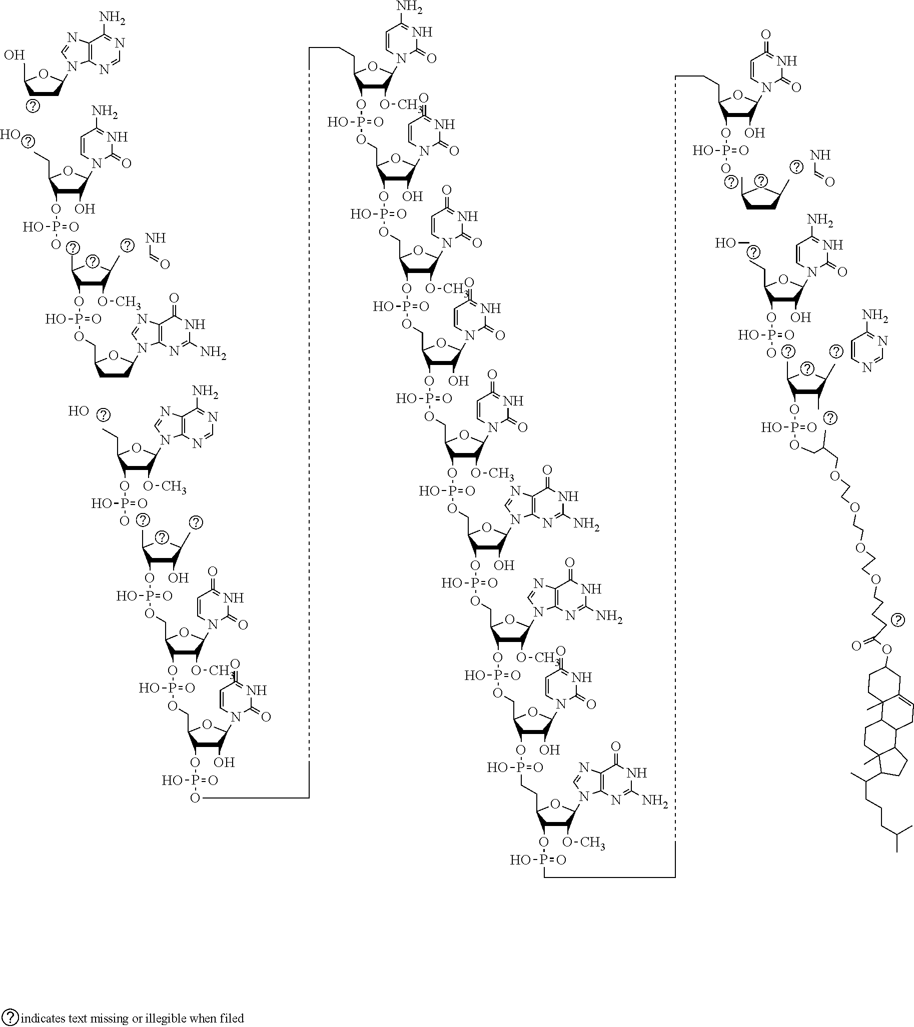

The structural formula of the CWT-001 passenger strand in ionised form (i.e., the anion of a salt) is shown below:

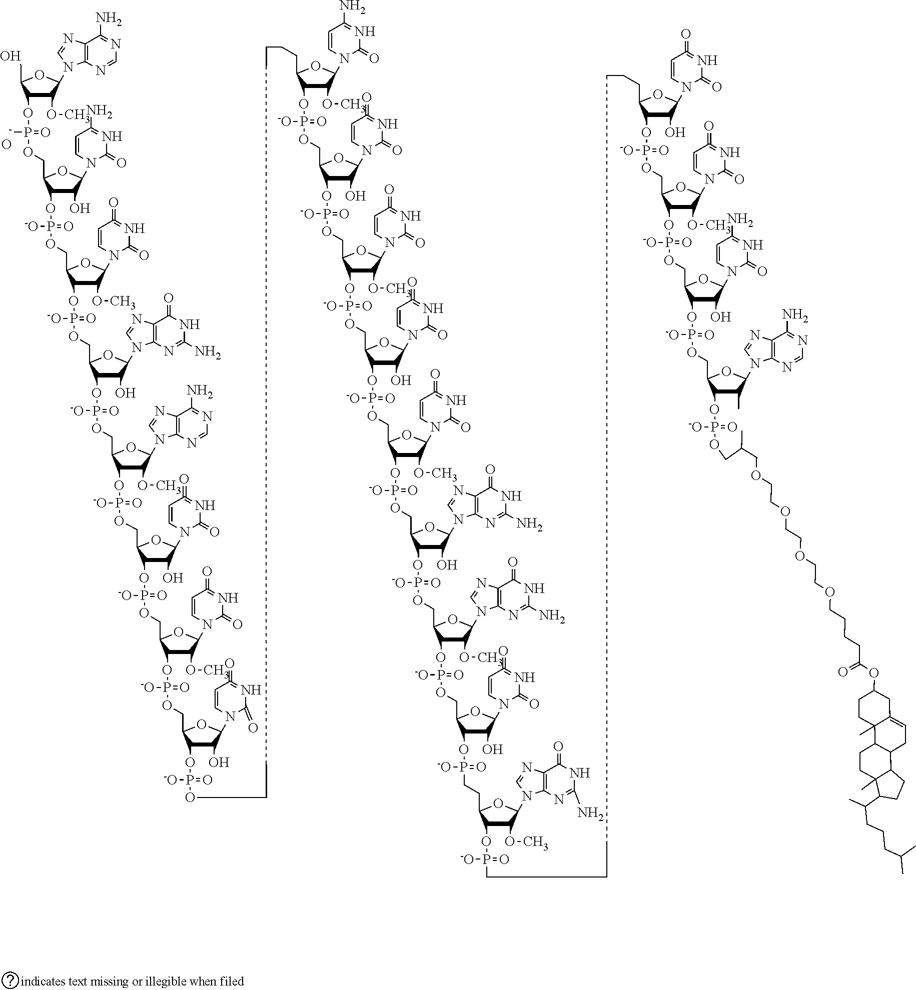

The structural formula of the CWT-001 passenger strand, as a sodium salt, is shown below and in FIG. 1B:

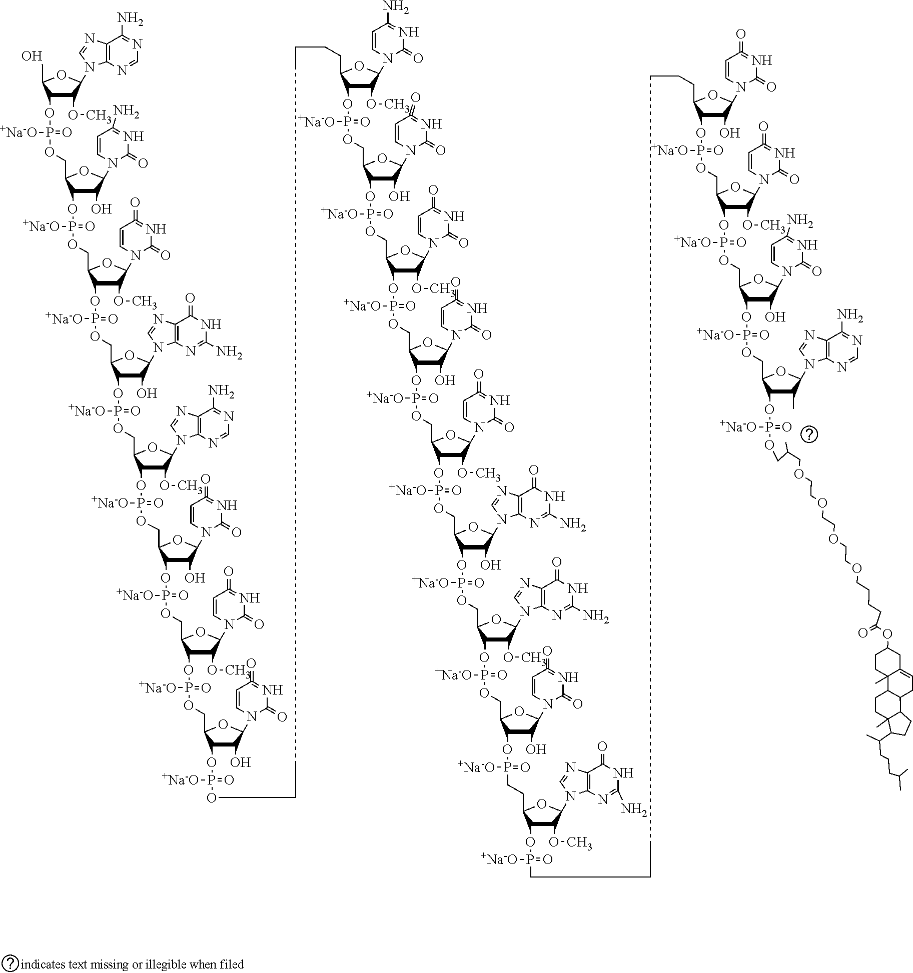

Further information on CWT-001 is provided in the following table A.

TABLE A

| | CWT-001 | CWT-001 |
|---|---|---|
| | antisense strand | sense strand |
| Structural | C₂₁₁H₂₄₄N₈₂O₁₃₄P₂₂F₂₀Na₂₂ | C₂₄₆H₃₃2N₆₇O₁₆₂P₂₁Na₂₁ |
| formula, | | |
| sodium salt | | |
| Molecular mass, | 7639.47 | 7952.02 |
| sodium salt | | |
| Molecular mass, | 7134.1 | 7470.1 |
| free acid | | |
| Molecular mass, | 7134.1 | 6714.2 |
| free acid | | |
| (excluding | | |
| cholesterol | | |
| moiety) | | |

Methods of Treatment and Therapeutic Uses of the microRNA-29 Compounds and Compositions

The compounds and compositions described herein are particularly suitable in the treatment of diseases that involve localised collagen dysregulation, characterised by a local overproduction of one or more types of collagen fibre. For example, the miR-29 compounds described herein reduce or inhibit the expression of collagen 3, for example as assessed in a luciferase assay. The miR-29 compounds described herein therefore may be useful in the treatment of diseases that require the reduction or inhibition of expression of one or more types of collagen fibres, for example collagen 3 (col3a1).

Three exemplary diseases are described below: tendon damage (in particular tendinopathy), Peyronie's disease and Dupuytren's disease, the latter two being examples of tissue fibrosis.

### Tendon Damage

Tendons are the connective tissue attaching muscle to bone. Tendon tissue comprises around 30% collagen and 2% elastin (wet weight) embedded in an extracellular matrix containing various types of cells, most notably tenocytes. The predominant collagen is type 1 collagen, which has a large diameter (40-60 nm) and links together to form tight fibre bundles. Type 3 collagen is also present and is smaller in diameter (10-20 nm), forming looser reticular bundles. A healthy tendon contains primarily well-ordered bundles of collagen 1 (col1) fibres (˜95%) interspersed with smaller amounts of biomechanically inferior collagen 3 (col3) fibres (˜5%). In healthy tenocytes, miR-29 (e.g., miR-29a) directly suppresses collagen 3 overproduction, neovascularisation, hyper-proliferation of tenocytes, and adhesion formation, by targeting Col3a1, VEGFA, AKT3 and TGF-B2, respectively. The sequence of miR-29a and its binding sites in these target mRNAs are conserved in mammalian species.

The biomechanical properties of tendon, especially its tensile strength, are related to cross-sectional area (i.e., thickness), collagen content, and the ratio between different types of collagen.

As noted above, a healthy tendon contains primarily well-ordered bundles of collagen 1 (col1) fibres (˜95%) interspersed with smaller amounts of biomechanically inferior collagen 3 (col3) fibres (˜5%). After acute injury, during tendinopathy, and during healing of tendon damage, a shift occurs in collagen synthesis, away from type 1 collagen toward type 3 collagen. A persistent increase in type 3 collagen synthesis leads to a long-term imbalance in collagen ratio. This has a significant and deleterious effect on the biomechanical properties of the tendon. In particular, it reduces the tensile strength of the tendon, reducing its ultimate failure strength and thus making it more prone to subsequent rupture.

Tendon damage may be caused by or associated with numerous factors including but not limited to external trauma, mechanical stress (including over-use), degeneration, inflammation, and combinations of these. Tendinopathy is the medical name for diseases associated with overuse and injury of the tendon.

Overuse tendinopathy is a complex multi-faceted pathology of the tendon, clinically diagnosed after gradual onset of activity-related pain, decreased function and sometimes with localized swelling of the tendon. Over-use, repetitive strain or trauma leads to the breakdown of collagen fibres in the tendon. In response to this damage, tenocytes initiate a repair response, where they initially produce high levels of collagen 3 which acts as a ‘patch’ to quickly restore tendon function. Over time collagen 3 will be gradually replaced by collagen 1. However, as collagen 3 is biomechanically inferior to collagen 1, the tendon is prone to further damage, causing more collagen 3 production. In tendinopathy, a vicious cycle of further damage and inferior healing results in the formation of lesions characterised by increased collagen 3 (˜30%), cellularity, vascularisation, inflammation and adhesion formation.

Conventional treatments for tendinopathy such as steroid injection are generally employed empirically to fight pain and inflammation but they do not modify the histological structure of the tendon. However, these treatments are not completely satisfactory and the recurrence of symptoms is common.

The examples that follow inter alia indicate that the miR-29 compounds provided herein are safe and effective in reducing or inhibiting collagen 3 expression in vivo, including in mouse models of tendinopathy. For example, using a mouse model, FIG. 4 shows that treatment with an exemplary miR-29 compound according to the invention (CWT-001) significantly improved tendon fibre alignment and collagen production via the suppression of collagen 3 expression in tendon disease. Additionally, the inventors have previously shown that an equine collagenase-induced tendinopathy (SDFT) model reflects human tendinopathy, with reduced expression of miR-29a after tendon injury and a concomitant increase in col3:col1 ratio. A single intralesional injection of a miR-29 compound reduced collagen 3 expression and lesion size, and improved tendon structure (see ref. 3). Together, these data provide a rationale for safe and effective human therapy using miR-29 compounds, including the miR-29 compounds provided herein. In light of the experiments described herein (see the Examples), doses in the μg and low mg range are expected to be well tolerated in animal species and can be administered in volumes that can easily and safely be injected (e.g., around 1 ml). In a phase 1, randomised, double-blind, placebo-controlled study in human subjects with lateral epicondylitis, doses of 200 μg, 500 μg and 1500 μg CWT-001 administered in a 1 ml volume were found to be safe and effective (see Example 7).

The miR-29 compounds and compositions of the invention may therefore be used in the treatment of tendinopathy, such as overuse tendinopathy. The subject may be a human subject. The subject may have sub-acute tendinopathy. Treatment with the compounds and compositions of this invention may reduce progression to chronic tendinopathy. The subject may have chronic tendinopathy. The subject may have active tendinopathy. The subject may have a partially-torn tendon. Treatment may reduce progression to a fully-torn tendon.

The methods of the invention may be applied to any damaged tendon. The main tendons affected by tendinopathy in humans are the Achilles tendon, the supraspinatus tendon, the common flexor tendons, the common extensor tendons, the patellar tendon and the gluteal tendons. The main tendon affected by tendinopathy in equine subjects is the superficial flexor tendon. In specific embodiments, one or more of these tendons are targets for treatment.

The tendon damage may therefore be lateral epicondylitis, medial epicondylitis, rotator cuff tendinopathy, common extensor origin tendinopathy, common flexor origin tendinopathy, gluteal tendinopathy (greater trochanteric pain syndrome, GTPS), patellar tendinopathy, jumper's knee, plantar fasciitis, Achilles tendinopathy, peroneal tendinopathy, supraspinatus syndrome, or a combination thereof. In one embodiment, the tendon damage is upper limb tendinopathy (including lateral epicondylitis, golfer's elbow, rotator cuff tendinopathy and De Quervain's disease). In a particular embodiment, the tendon damage is lateral epicondylitis.

Treatment with a miR-29 compound promotes the regeneration or repair of the afflicted tissue, e.g., in the tendon, tunica albuginea or plantar fascia.

Treatment with a miR-29 compound reduces the expression of collagen, e.g., collagen 3, at the site of treatment.

The methods of the invention may be applied at any stage of tendon damage, or at any stage of the healing process of a damaged tendon. In one embodiment, the miR-29 compounds and compositions of the invention are administered to the subject at the point of initial diagnosis.

The subject, for example the human subject, may have failed to respond to, or had an inadequate response to, or was intolerant to a prior treatment. This means that the subject's symptoms have persisted despite the treatment. A prior tendinopathy treatment may include one or more of: physical therapy, splinting, treatment with a nonsteroidal anti-inflammatory drug (NSAID), and local corticosteroid injection into the affected tendon. The subject may have failed conservative treatment, i.e., the symptoms persisted despite treatment, for a period of 3 months or more.

Efficacy of miR-29 treatment in tendon damage (e.g., tendinopathy, such as elbow tendinopathy) can be assessed on the basis of one or more of (see also Example 7):
reduction of pain, for example measured using Visual Analogue Scale (VAS), e.g., at 14 days, 28 days and/or 90 days following administration of a miR-29 compound or salt thereof;
improvement in disability and symptoms, for example measured using the Disabilities of the Arm, Shoulder, and Hand (Quick DASH) Score, and/or the American Shoulder and Elbow Surgeons Elbow (ASES-E), e.g., at 14 days, 28 days and/or 90 days following administration of a miR-29 compound or salt thereof;
improvement in pain and disability, for example measured by the Patient Rated Tennis Elbow Evaluation (PRTEE), e.g., at 14 days, 28 days and/or 90 days following administration of a miR-29 compound or salt thereof; and
improvement in the treated tendon(s) as measured by ultrasound assessment, e.g., at 14 days, 28 days and/or 90 days following administration of a miR-29 compound or salt thereof.

### Dupuytren's Disease

Dupuytren's disease (DD) affects the fibrous layer of tissue that lies underneath the skin in the palm and fingers, also known as the ‘fascia’ or ‘palmar fascia’. In subjects with DD, the fascia thickens, then tightens over time. This causes the fingers to be pulled inward, towards the palm, resulting in what is known as a Dupuytren's contracture. Subjects with DD may be unable to perform certain daily activities. Although nonsurgical (e.g., steroid or collagenase injection) and surgical treatment options are available to help slow the progression of the disease and improve motion in the affected fingers, these can have significant side effects or complications.

On a molecular level, DD is characterised by an abnormal fibroblast proliferation process and an overproduction of collagens, including collagen 3 (ref. 6). The miR-29 compounds described herein are thought to be efficacious in the treatment of Dupuytren's disease on the basis of their ability to inhibit particularly effectively and safely local collagen (e.g., collagen 3) expression in the fascia, therefore countering the formation of fibrotic tissue that characterises this disease.

Efficacy of miR-29 treatment in Dupuytren's disease can be assessed on the basis of improvement in disability and symptoms, for example measured using the Disabilities of the Arm, Shoulder, and Hand (Quick DASH) Score, symptoms and/or reduction of pain (VAS scoring), and clinical assessment of reduction in Dupuytren's nodule and cord formation (for example by improvement in the Tubiana grading system). See, e.g., ref. 7.

### Peyronie's Disease

Peyronie's disease (PD) is a progressive fibrotic disorder characterised by the formation of scars or plaques within the tunica albuginea (TA) of the penis. The condition is highly debilitating on both a physical as well as an emotional level due to penile pain and the development of penile deformities. Excessive local accumulation of collagen 3 and other components of the extracellular matrix are characteristic of the disease. Fibrotic tissue is associated with a shift in the normal collagen 3/1 ratio toward an increase in the content of collagen 3. For example, it has been shown that PD animals developed tunical and subtunical areas of fibrosis with a significant upregulation of collagen 3 protein. Furthermore, the collagen 3/1 ratio was higher in the PD group compared with control groups (P<0.05) (ref. 8). The miR-29 compounds described herein are thought to be efficacious in the treatment of Peyronie's disease on the basis of their ability to inhibit particularly effectively and safely local collagen 3 expression in the TA, therefore countering the formation of fibrotic tissue that characterises this disease.

Co-occurrence of Dupuytren's disease and Peyronie's disease is common (e.g., ref. 9) and both diseases are associated with collagen 3 overproduction (ref. 10). The data suggest that they share a common pathophysiology and are amenable to the same therapeutic regimens. This further supports the therapeutic efficacy of miR-29 mimics including those provided herein in the treatment of Peyronie's disease and Dupuytren's disease.

Efficacy of miR-29 treatment in Peyronie's disease can be assessed on the basis of improvement in disability, symptoms and/or reduction of pain, improvement or lack of progression of penile curvature and improvement in duplex ultrasonography.

### Subjects for Treatment

The subject is typically a human. However, the methods of the invention may extend to any other mammals, including other primates (especially great apes such as gorilla, chimpanzee and orangutan, but also Old World and New World monkeys) as well as rodents (including mice and rats), and other common laboratory, domestic and agricultural animals (including but not limited to rabbits, dogs, cats, horses, cows, sheep, goats, etc.). Another preferred subject is a horse, particularly a horse that has been bred for equine sport.

### Pharmaceutical Compositions and Administration

The miR-29 compounds or salts thereof described herein can be formulated in pharmaceutical compositions. These compositions may comprise, in addition to the miR-29 compound or salt thereof, a pharmaceutically acceptable excipient, carrier, buffer, stabiliser or other materials well known to those skilled in the art. Such materials are non-toxic and do not interfere with the efficacy of the miR-29 compound.

Thus in one embodiment the pharmaceutical composition comprises a miR-29 compound or a salt thereof and a pharmaceutically acceptable carrier or diluent, such as a sterile liquid carrier, e.g., an injectable solution. The pharmaceutically acceptable carrier or diluent may comprise saline. The pharmaceutically acceptable carrier or diluent may be a sterile aqueous solution, for example phosphate buffered saline (PBS). The pharmaceutically acceptable carrier or diluent may be a sterile aqueous solution, for example a sodium chloride solution such as isotonic saline (e.g., 0.9% w/v NaCl). In one embodiment, the pharmaceutical composition comprises a miR-29 compound or a salt thereof, and further comprises PBS and a sodium chloride solution such as isotonic saline (e.g., 0.9% w/v NaCl).

In view of the localised nature of the conditions to be treated, local administration, for example by local injection, is particularly suitable. The compounds and compositions of the invention are therefore typically administered locally at the site of affliction (intra-lesionally). For example, the injection may be delivered into the affected tendon. Injection into the affected tendon is also known as intratendinous administration.

For injection at the site of affliction, the miR-29 compound can be comprised in a pharmaceutical composition further comprising a parenterally acceptable aqueous solution. The solution is sterile. The solution is pyrogen-free and has suitable pH, isotonicity and stability. Those of skill in the art are well able to prepare suitable solutions using, for example, isotonic vehicles such as phosphate buffered saline, sodium chloride solution, Ringer's solution, Lactated Ringer's solution. Preservatives, stabilisers, buffers, antioxidants and/or other additives may be included, as required. For example, EDTA may added, e.g., at a final concentration of 0.001%-0.005%, e.g., 0.003% (100 PM). A particular formulation of the compounds of the invention is an injectable solution, for example a solution comprising a miR-29 compound or salt of the invention and PBS, optionally further comprising EDTA.

Injection into the tendon is typically guided by ultrasound, for example using the “peppering technique” (ref. 11). The “peppering technique” is an injection method whereby multiple small injections are performed by withdrawing, redirecting and reinserting the needle without emerging from the skin. In the injured/chronic tendon situation this may be advantageous in order to stimulate a healing response. Thus in one embodiment, a single dose of the miR-29 compound is administered into the tendon in the form of multiple needle injections at different sites within the damaged tendon.

Examples of routine techniques and protocols can be found in Remington's Pharmaceutical Sciences, 22nd Edition, 2012.

### Dosages and Treatment Regimens

The person skilled in the art is aware that dosages can be expressed in various way. Unless otherwise indicated, a dose of a miR-29 compound as indicated herein refers to the weight of the oligonucleotide portion of the guide and passenger strand (i.e., excluding any conjugate groups such as a cholesterol moiety), as a free acid. The reference compound is CWT-001, as a free acid, and excluding the triethyleneglycol cholesterol moiety. Equivalent doses of miR-29 compounds that may have a different molecular weight, of salts, and/or of conjugated forms, can be calculated by the skilled person. Equivalent doses may also be indicated in molar amounts (e.g., nmoles) or molar concentrations (e.g., nM). The skilled person knows how to convert between these different units.

In the Examples and Figures described below, the indicated dosages refer to the free acid of the cholesterol-conjugated form of the compounds. Therefore, where the Examples and Figures refer to 200 μg or 200 μg/ml, this corresponds to about 190 μg or 190 μg/ml of the compound as a free acid excluding the cholesterol moiety; where the Examples refer to 500 μg or 500 μg/ml, this corresponds to about 475 μg or 475 μg/ml of the compound as a free acid excluding the cholesterol moiety; where the Examples refer to 1500 μg or 1500 μg/ml, this corresponds to about 1420 μg or 1420 μg/ml of the compound as a free acid excluding the cholesterol moiety; where the Examples refer to 2000 μg or 2000 μg/ml, this corresponds to about 1900 μg or 1900 μg/ml of the compound as a free acid excluding the cholesterol moiety, etc. Where the cholesterol moiety is excluded, this excludes also any linkers (e.g., triethyleneglycol) between the cholesterol moiety and the oligonucleotide portions of the compound.

Administration is typically in a “therapeutically effective amount” or a “prophylactically effective amount” (as the case may be, although therapy may comprise prophylaxis), this being sufficient to show benefit to the subject. For example, a therapeutically effective amount is an amount of the miR-29 compound or salt thereof that when administered to a subject results in an improvement in disease symptoms, e.g., reduced pain or disability, improved functionality, and/or prevented or slower disease progression.

A miR-29 compound can be administered directly into the damaged tissue, i.e., intralesionally. Administration is typically by injection, for example using a needle.

In one embodiment, a therapeutic effect may be achieved with only a single administration of the doses described herein. A “single” administration means that only one dose of the compound is required to achieve a therapeutic effect. The subject therefore typically does not need to return for a second dose in order to experience an improvement in disease symptoms at the site of administration of the first dose.

### General

The practice of the present invention will employ, unless otherwise indicated, conventional methods of chemistry, biochemistry, molecular biology, immunology and pharmacology, within the skill of the art.

The term “comprising” encompasses “including” as well as “consisting”, e.g., a composition “comprising” X may consist exclusively of X or may include something additional, e.g., X+Y.

The term “about” in relation to a numerical value x is optional and means, for example, x±10%.

The word “substantially” does not exclude “completely”. Where necessary, the word “substantially” may be omitted from the definition of the invention.

References to a percentage sequence identity between two oligonucleotide sequences means that, when aligned, that percentage of nucleobases is the same in comparing the two sequences, across the full length of those sequences.

The following examples are provided to illustrate various embodiments of the present invention. The examples are illustrative and are not intended to limit the invention in any way.

### BRIEF DESCRIPTION OF THE DRAWINGS

FIG. 1: (A) Structure of the CWT-001 guide (antisense) strand, as a sodium salt. (B) Structure of the CWT-001 passenger (sense) strand, as a sodium salt.

FIG. 2: Luciferase activity in HEK293 cells co-transfected with luciferase plasmid containing human collagen 3 miR29-binding sites and miR29a compounds and controls as indicated (n=4). Luciferase activity is inversely proportional to miR29a activity.

FIG. 3: (A) Selected miR29 compounds were tested for potential immunogenicity by assessing their capacity to induce the production of pro-inflammatory cytokine TNF-alpha in primary human macrophages. Untreated cells (“untreated”) were used as negative control; cells treated with 2.5 μg/ml imiquimod (“IMQ 2.5”) were used as a positive control. (B) Selected miR29 compounds were tested for their ability to knock-down the expression of col3a1 in human tenocytes. Scrambled miR29a (“scr”), and untreated cells (“untr”) were used as a negative control. (C) Luciferase activity of HEK293 cells co-transfected with luciferase plasmid containing human collagen 3 miR29-binding sites and selected miR-29 compounds as indicated. Scrambled miR29a (“scr”) was used as a negative control. Significance was assessed using one-way ANOVA test. ***P<0.001 compared to Teno20, n=3. (D) Col3a1 transcript levels measured by quantitative PCR following treatment of tenocytes with cholesterol-conjugated miR-29 compounds as indicated. Values normalised to GAPDH and shown as relative expression compared to untreated samples. Student's t-test, *P<0.05 Teno18 compared to Teno33 at 5 days, n=3. (E) Selected miR-29 compounds were tested in a mouse tendon injury model. The compounds were injected directly into injured patellar tendons and the mice were allowed to recover for 1, 3 or 7 days. The absolute numbers of Col3a1 transcripts were measured by q-PCR. Significance was assessed using one-way ANOVA test. *P<0.05, **P<0.01 Teno18 compared to Teno20 at each time point, n=4.

FIG. 4: Histological and molecular analysis of mouse tendons following induction of tendon injury followed immediately by treatment with CWT-001. (A) Absolute copy number of col3a1 mRNA in mouse patellar tendon on days 1, 3 and 7 post-injection of CWT-001 or PBS (n=3). (B) Representative H&E stained sections of injured tendons after 7 days of treatment with CWT-001 (right) or PBS control (left).

FIG. 5: Levels of pro-inflammatory cytokine tumour necrosis factor alpha (TNF-α) in the supernatants of cultured primary human macrophages 24 hours after treatment with CWT-001, scrambled negative control or poly IC positive control, measured by ELISA.

FIG. 6: Expression levels of interferon-inducible genes OAS2 (A), INFa (B), IFTM1 (C), MX1 (D), IRF9 (E) and OAS1 (F) in cultured primary human macrophages 24 hours after treatment with CWT-001 (middle bar) or poly IC positive control (right bar), measured by qPCR and expressed as fold change relative to untreated control (left bar). “no TF”=no transfection reagent (cellular uptake was enhanced by the cholesterol moiety).

FIG. 7: Expression levels of interferon-inducible genes MX1 (A), OAS2 (B), IFITM1 (C) and IRF9 (D) in mouse tendon at 0, 1, 3 and 7 days after intra-tendon injection of CWT-001 or PBS negative control, measured by qPCR and expressed as absolute copy number of interferon response genes per 106 copies of 18S.

FIG. 8: Levels of miR29a in fresh human serum incubated at 37° C. with 0, 2, 20 and 100 μg/ml CWT-001 for 0, 6, 12 and 24 hours, measured by qPCR normalised to Cel39 spike in control and shown as relative expression compared to untreated samples (n=3).

FIG. 9: Levels of miR29a in fresh human serum incubated at 37° C. with 0, 2, 20 and 100 μg/ml CWT-001 for 0 minutes, 15 minutes, 30 minutes, 45 minutes, 1 hour, 6 hours, 12 hours and 24 hours, measured by qPCR normalised to Cel39 spike in control and expressed as fold-change relative to untreated samples (n=3).

FIG. 10: Levels of miR29a in mouse kidney (A), spleen (B), liver (C), serum (D), lung (E), muscle (F) and heart (G) harvested 0, 1, 4 and 7 days after intravenous injection of CWT-001, measured by qPCR. Organ miR29a expression levels were normalised to GAPDH and shown as relative fold-change compared to untreated controls, serum miR29a expression levels were normalised to Cel39 spike in control and expressed as fold-change relative to PBS control (n=4).

FIG. 11: Serum levels of miR29a in blood collected from mice 0 minutes, 15 minutes, 30 minutes, 1 hour, 2 hours, 4 hours, 6 hours, 24 hours and 72 hours after injection of CWT-001 into the patella tendon, measured by qPCR normalised to Cel39 spike in control and expressed as fold-change relative to PBS-treated control (n=4).

FIG. 12: Levels of miR29a in mouse serum (A), lung (B), skin proximal to tendon injection (C), kidney (D), spleen (E), muscle (F), liver (G), heart (H) and bone marrow (I) harvested 7 days after intravenous injection of 4, 20, 40 and 50 mg/ml of CWT-001, or PBS control, measured by qPCR. Organ miR29a expression levels were normalised to GAPDH and shown as relative fold-change compared to untreated controls, serum miR29a expression levels were normalised to Cel39 spike in control and expressed as fold-change relative to PBS control (n=4).

FIG. 13: Study design for a phase 1, randomised, double-blind, placebo-controlled study evaluating the safety, tolerability and pharmacokinetics of single ascending doses of CWT-001 injections in human subjects with lateral epicondylitis.

FIG. 14: Ultrasound images (A and B) illustrating probe placement during administration of CWT-001 to the common extensor tendon. The radial head (r), radiohumeral joint (j), lateral epicondyle, and common extensor tendon are visualised. The probe is placed along the lateral aspect of the elbow parallel to the longitudinal axis of the common extensor tendon.

FIG. 15: Comparison of levels of miR29a in healthy and Dupuytren's human fascia tissue, measured by qPCR (n=4 healthy, n=3 Dupuytren's).

FIG. 16: Levels of collagen 1 (A) and collagen 3 (B) in human Dupuytren's fibroblasts following transfection with 5 μM of a miR29a mimic, a miR29b mimic, a miR29c mimic, CWT-001, a miR29a antagomir or a scrambled miR29a control, measured by qPCR. miR29a expression levels normalised to GAPDH and shown as relative fold-change compared to scrambled controls (n=3). *P<0.05, **P<0.01, ***P<0.005 versus scrambled control. (Student's t-test).

FIG. 17: In a phase I randomised, double-blind study, human subjects with lateral epicondylitis received a single 200 μg (n=6) (A), 500 μg (n=6) (B) or 1500 μg (n=6) (C) dose of CWT-001 or matched placebo (n=2) (3:1) administered via intra-tendon injection. The subjects assessed their pain using 100 mm visual analogue scale (VAS) once per day for the first 14 days post treatment, rating the pain during the previous 24 hours. After Day 14 the subjects kept a weekly pain diary until day 90 post injection. The horizontal “MCID” line indicates the established minimal clinically importance difference (MCID) for VAS in the treatment of lateral epicondylitis. The mean change in VAS score for all subjects that received any dose of CWT-001 (n=18) was compared with the mean change in VAS score for all subjects that received the placebo (n=6) (D), and the mean percentage change in VAS score for all subjects that received any dose of CWT-001 was compared with the mean percentage change in VAS score for subjects that were treated with corticosteroid (n=50), platelet rich plasma (PRP) (n=50), or xylocaine (n=50), in a separate study (E).

FIG. 18: In a phase I randomised, double-blind study, human subjects with lateral epicondylitis received a single 200 μg (n=6) (A), 500 μg (n=6) (B) or 1500 μg (n=6) (C) dose of CWT-001 or matched placebo (n=2) (3:1) administered via intra-tendon injection. Subjects reported their physical function and symptoms using the QuickDASH index, and the change in QuickDASH score was plotted from pre-dose on Day 1, to Day 14, Day 28 and Day 90. The mean change in QuickDASH score for all subjects that received any dose of CWT-001 (n=18) was compared with the mean change in QuickDASH score for all subjects that received the placebo (n=6) (D). The horizontal “MCID” line indicates the established MCID for QuickDASH in patients with upper-limb musculoskeletal disorders.

FIG. 19: In a phase I randomised, double-blind study, human subjects with lateral epicondylitis received a single 200 μg (n=6) (A), 500 μg (n=6) (B) or 1500 μg (n=6) (C) dose of CWT-001 or matched placebo (n=2) (3:1) administered via intra-tendon injection. The patient rated tennis elbow evaluation (PRTEE) was used to measure perceived pain and disability in study subjects, and the change in PRTEE score was plotted from pre-dose on Day 1, to Day 14, Day 28 and Day 90. The mean change in PRTEE score for all subjects that received any dose of CWT-001 (n=18) was compared with the mean change in PRTEE score for all subjects that received the placebo (n=6) (D).

FIG. 20: In a phase I randomised, double-blind study, human subjects with lateral epicondylitis received a single 200 μg (n=6), 500 μg (n=6) or 1500 μg (n=6) dose of CWT-001 or matched placebo (n=2) (3:1) administered via intra-tendon injection. Ultrasound tissue characterisation (UTC) was used to visualise tendon structure and to quantify grey-scale tendon matrix changes into 4 different echotypes related to tendon integrity. Types I and II represent an organised matrix; Types III and IV represent a disorganised matrix (A). Representative ultrasound images of the lateral elbow are provided for a subject that had received the placebo at pre-injection (B), Day 28 (C) and Day 90 (D), and for a subject that had received 200 μg CWT-001 at pre-injection (E), Day 20 (F) and Day 90 (G). The percentages of UTC Type I, II, III and IV tendon tissue and the total percentage of reparative tendon (UTC Types I and II) vs degenerative tendon (UTC types III and IV) were quantified for the subjects that had received a 200 μg (H, I), 500 μg (J, K) or 1500 μg (L, M) dose of CWT-001 or matched placebo. The mean percentages of reparative and degenerative tendon for all subjects that received any dose of CWT-001 (n=18) was compared with the mean percentages of reparative and degenerative tendon for all subjects that received the placebo (n=6) (N). *P<0.05, **P<0.01.

### EXAMPLES

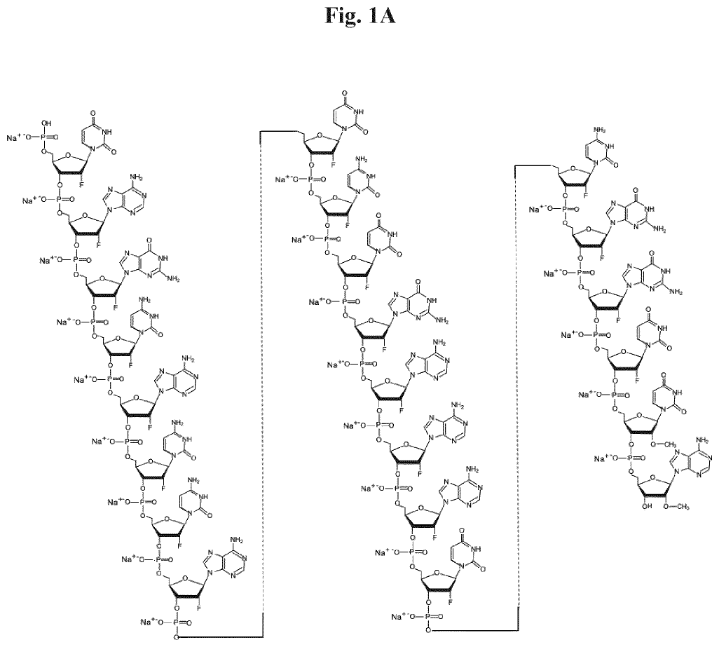

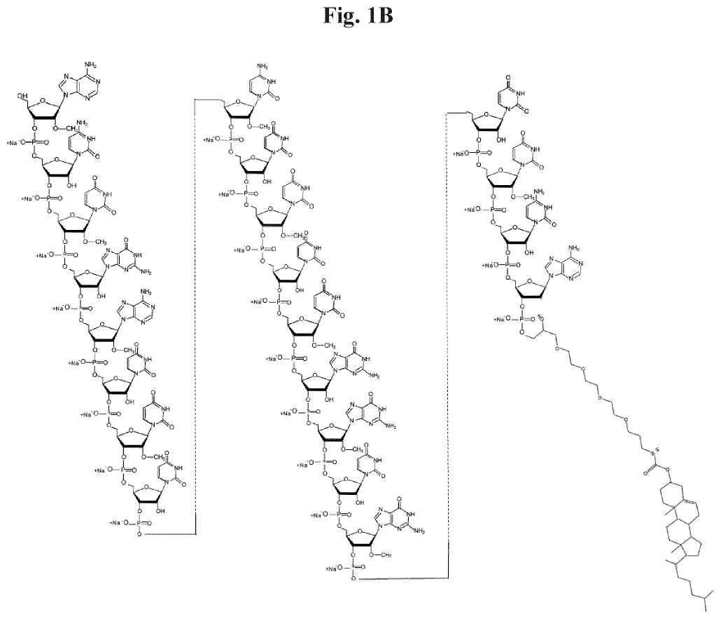

### Example 1: Evaluation of miR-29 Compounds and Primary Pharmacology Studies (FIGS. 1-3)

A library of miR-29 mimic compounds containing various chemical modifications (2′-fluoro ribose, 2′-O-Methyl ribose, deoxyribose, and/or phosphorothioate) in different combinations was created. All compounds with the exception of one (Teno36) are double-stranded compounds containing a guide strand and a passenger strand. The guide strand of each compound has a nucleobase sequence that is identical to the guide strand of natural human mature miR-29a (hsa-miR-29a-3p; SEQ ID NO: 1). The passenger strand has a nucleobase sequence that is either identical to the passenger strand of natural human mature miR-29a (hsa-miR-29a-5p; SEQ ID NO: 2), or it may lack the 3′ G nucleotide (SEQ ID NO: 3), and/or it may have one or more nucleotide substitutions relative to the sequence of the natural miR-29a passenger strand (SEQ ID NOs: 4-6), see Table 1b. A negative control passenger strand was also tested, representing the reverse sequence of SEQ ID NO: 2 (SEQ ID NO: 7). The nucleobase sequences together with any sugar and internucleoside linkage (backbone) modifications are shown in Table 1a. For each compound, the first row indicates the guide strand, the second row indicates the passenger strand. All sequences are shown in the 5′ to 3′ direction. The SEQ ID NOs indicate the nucleobase sequence (i.e., excluding the chemical modifications). Teno46 does not contain any chemical modifications and therefore corresponds to native mature miR-29a. Teno39 was previously described (ref. 2). The structure of an exemplary compound, Teno18, as a sodium salt and wherein the passenger strand is conjugated at the 3′ end to a cholesterol moiety, is shown in FIG. 1A (guide strand) and 1B (passenger strand).

TABLE 1a
miR-29 compounds

| | | Nucleo- |
|---|---|---|
| | | base |
| Compound | Formula | SEQ ID NO |
| Teno1 | /5Phos/rUrArGrCrArCrCrArUrCrUrGrArArArUrCrGrGmUmUmA | 1 |
| | mArCmUrGmArUmUrUmCrUmUrUmUrGmGrUmGrUmUrCmArG | 2 |
| Teno2 | /5Phos/rUrArGrCrArCrCrArUrCrUrGrArArArUrCrGrGmUmUmA | 1 |
| | mArCmCrGmArUmUrUmCrAmArUmUrGmGrUmGrCmUrAmG | 4 |
| Teno3 | /5Phos/rUrArGrCrArCrCrArUrCrUrGrArArArUrCrGrGmUmUmA | 1 |
| | rGrArCrUrUrGrUrGrGrUrUrUrUrCrUrUrUrArGrUrCrA | 7 |
| Teno4 | /5Phos/rUrArGrCrArCrCrArUrCrUrGrArArArUrCrGrGmUmUmA | 1 |
| | mArCmUrGmArUmUrUmCrUmUrUmUrGmGrUmGrUmUrCA | 3 |
| Teno5 | /5Phos/rUrArGrCrArCrCrArUrCrUrGrArArArUrCrGrGmUmUmA | 1 |
| | mArCmUrGmArUmUrUmCrUmUrUmUrGmGrUmGrUmUrCmAG | 2 |
| Teno6 | /5Phos/rUrArGrCrArCrCrArUrCrUrGrArArArUrCrGrGmUmUmA | 1 |
| | mAmCrUmGrAmUrUmUrCmUrUmUrUmGrGmUrGmUrUmCmAmG | 2 |
| Teno7 | /5Phos/rUrArGrCrArCrCrArUrCrUrGrArArArUrCrGrGmUmUmA | 1 |
| | mAmCrCmGrAmUrUmUrCmArAmUrUmGrGmUrGmCrUmAmG | 4 |
| Teno8 | /5Phos/rUrArGrCrArCrCrArUrCrUrGrArArArUrCrGrGrU*rU*rA | 1 |
| | mArCmUrGmArUmUrUmCrUmUrUmUrGmGrUmGrUmUrCmArG | 2 |
| Teno9 | /5Phos/rUrArGrCrArCrCrArUrCrUrGrArArArUrCrGrGrU*rU*rA | 1 |
| | mArCmCrGmArUmUrUmCrAmArUmUrGmGrUmGrCmUrAmG | 4 |
| Teno10 | /5Phos/rUrArGrCrArCrCrArUrCrUrGrArArArUrCrGrGrU*rU*rA | 1 |
| | rGrArCrUrUrGrUrGrGrUrUrUrUrCrUrUrUrArGrUrCrA | 7 |
| Teno11 | /5Phos/rUrArGrCrArCrCrArUrCrUrGrArArArUrCrGrGrU*rU*rA | 1 |
| | mArCmUrGmArUmUrUmCrUmUrUmUrGmGrUmGrUmUrCA | 3 |
| Teno12 | /5Phos/rUrArGrCrArCrCrArUrCrUrGrArArArUrCrGrGrU*rU*rA | 1 |
| | mArCmUrGmArUmUrUmCrUmUrUmUrGmGrUmGrUmUrCmAG | 2 |
| Teno13 | /5Phos/rUrArGrCrArCrCrArUrCrUrGrArArArUrCrGrGrU*rU*rA | 1 |
| | mAmCrUmGrAmUrUmUrCmUrUmUrUmGrGmUrGmUrUmCmAmG | 2 |
| Teno14 | 5Phos/rUrArGrCrArCrCrArUrCrUrGrArArArUrCrGrGrU*rU*rA | 1 |
| | mAmCrCmGrAmUrUmUrCmArAmUrUmGrGmUrGmCrUmAmG | 4 |
| Teno15 | /5Phos/fUfAfGfCfAfCfCfAfUfCfUfGfAfAfAfUfCfGfGfUmUmA | 1 |
| | mArCmUrGmArUmUrUmCrUmUrUmUrGmGrUmGrUmUrCmArG | 2 |
| Teno16 | /5Phos/fUfAfGfCfAfCfCfAfUfCfUfGfAfAfAfUfCfGfGfUmUmA | 1 |
| | mArCmCrGmArUmUrUmCrAmArUmUrGmGrUmGrCmUrAmG | 4 |
| Teno17 | /5Phos/fUfAfGfCfAfCfCfAfUfCfUfGfAfAfAfUfCfGfGfUmUmA | 1 |
| | rGrArCrUrUrGrUrGrGrUrUrUrUrCrUrUrUrArGrUrCrA | 7 |
| Teno18 | /5Phos/fUfAfGfCfAfCfCfAfUfCfUfGfAfAfAfUfCfGfGfUmUmA | 1 |
| | mArCmUrGmArUmUrUmCrUmUrUmUrGmGrUmGrUmUrCA | 3 |
| Teno19 | /5Phos/fUfAfGfCfAfCfCfAfUfCfUfGfAfAfAfUfCfGfGfUmUmA | 1 |
| | mArCmUrGmArUmUrUmCrUmUrUmUrGmGrUmGrUmUrCmAG | 2 |
| Teno20 | /5Phos/fUfAfGfCfAfCfCfAfUfCfUfGfAfAfAfUfCfGfGfUmUmA | 1 |
| | mAmCrUmGrAmUrUmUrCmUrUmUrUmGrGmUrGmUrUmCmAmG | 2 |
| Teno21 | /5Phos/fUfAfGfCfAfCfCfAfUECEUEGEAfAfAfUECEGEGEUmUmA | 1 |
| | mAmCrCmGrAmUrUmUrCmArAmUrUmGrGmUrGmCrUmAmG | 4 |
| Teno22 | /5Phos/rUrArGrCrArCrCrArUrCrUrGrArArArUrCrGrGrUrUrA | 1 |
| | mArCmUrGmArUmUrUmCrUmUrUmUrGmGrUmGrUmUrCmArG | 2 |
| Teno23 | /5Phos/rUrArGrCrArCrCrArUrCrUrGrArArArUrCrGrGrUrUrA | 1 |
| | mArCmCrGmArUmUrUmCrAmArUmUrGmGrUmGrCmUrAmG | 4 |
| Teno24 | /5Phos/rUrArGrCrArCrCrArUrCrUrGrArArArUrCrGrGrUrUrA | 1 |
| | rGrArCrUrUrGrUrGrGrUrUrUrUrCrUrUrUrArGrUrCrA | 7 |
| Teno25 | /5Phos/rUrArGrCrArCrCrArUrCrUrGrArArArUrCrGrGrUrUrA | 1 |
| | mArCmUrGmArUmUrUmCrUmUrUmUrGmGrUmGrUmUrCA | 3 |
| Teno26 | /5Phos/rUrArGrCrArCrCrArUrCrUrGrArArArUrCrGrGrUrUrA | 1 |
| | mArCmUrGmArUmUrUmCrUmUrUmUrGmGrUmGrUmUrCmAG | 2 |
| Teno27 | /5Phos/rUrArGrCrArCrCrArUrCrUrGrArArArUrCrGrGrUrUrA | 1 |
| | mAmCrUmGrAmUrUmUrCmUrUmUrUmGrGmUrGmUrUmCmAmG | 2 |
| Teno28 | /5Phos/rUrArGrCrArCrCrArUrCrUrGrArArArUrCrGrGrUrUrA | 1 |
| | mAmCrCmGrAmUrUmUrCmArAmUrUmGrGmUrGmCrUmAmG | 4 |
| Teno29 | /5Phos/fUrArGfCrAfCfCrAfUfCfUrGrArArAfUfCrGrGfUfUmA | 1 |
| | mArCmUrGmArUmUrUmCrUmUrUmUrGmGrUmGrUmUrCmArG | 2 |
| Teno30 | /5Phos/fUrArGfCrAfCfCrAfUfCfUrGrArArAfUfCrGrGfUfUmA | 1 |
| | mArCmCrGmArUmUrUmCrAmArUmUrGmGrUmGrCmUrAmG | 4 |
| Teno31 | /5Phos/fUrArGfCrAfCfCrAfUfCfUrGrArArAfUfCrGrGfUfUmA | 1 |
| | rGrArCrUrUrGrUrGrGrUrUrUrUrCrUrUrUrArGrUrCrA | 7 |
| Teno32 | /5Phos/fUrArGfCrAfCfCrAfUfCfUrGrArArAfUfCrGrGfUfUmA | 1 |
| | mArCmUrGmArUmUrUmCrUmUrUmUrGmGrUmGrUmUrCA | 3 |
| Teno33 | /5Phos/fUrArGfCrAfCfCrAfUfCfUrGrArArAfUfCrGrGfUfUmA | 1 |
| | mArCmUrGmArUmUrUmCrUmUrUmUrGmGrUmGrUmUrCmAG | 2 |
| Teno34 | /5Phos/fUrArGfCrAfCfCrAfUfCfUrGrArArAfUfCrGrGfUfUmA | 1 |
| | mAmCrUmGrAmUrUmUrCmUrUmUrUmGrGmUrGmUrUmCmAmG | 2 |
| Teno35 | /5Phos/fUrArGfCrAfCfCrAfUfCfUrGrArArAfUfCrGrGfUfUmA | 1 |
| | mAmCrCmGrAmUrUmUrCmArAmUrUmGrGmUrGmCrUmAmG | 4 |
| Teno36 | /5Phos/fUfAfGCfAfCfCfAfUfCfUfGfAfAfAfUfCfGfGfUmUmA | 1 |
| | No passenger strand | n/a |
| Teno37 | /5Phos/rUrArGrCrArCrCrArUrCrUrGrArArArUrCrGrGmUmUmA | 1 |
| | mAmCrCmGrAmUrUmUrCmArGmArUmGrGmUrGmCrUA | 6 |
| Teno38 | /5Phos/rUrArGrCrArCrCrArUrCrUrGrArArArUrCrGrGmUmUmA | 1 |
| | mAmCrCmGrAmUrUmUrCmArGmArUmGrGmUrGmCrUmAmG | 5 |
| Teno39 | /5Phos/rUrArGrCrArCrCrArUrCrUrGrArArArUrCrGrGmUmUmA | 1 |
| | mAmCrCmGrAmUrUmUrCmArGmArUmGrGmUrGmCrUmAG | 5 |
| Teno40 | /5Phos/rUrArGrCrArCrCrArUrCrUrGrArArArUrCrGrGrU*rU*rA | 1 |
| | mAmCrCmGrAmUrUmUrCmArGmArUmGrGmUrGmCrUA | 6 |
| Teno41 | /5Phos/rUrArGrCrArCrCrArUrCrUrGrArArArUrCrGrGrU*rU*rA | 1 |
| | mAmCrCmGrAmUrUmUrCmArGmArUmGrGmUrGmCrUmAmG | 5 |
| Teno42 | /5Phos/rUrArGrCrArCrCrArUrCrUrGrArArArUrCrGrGrU*rU*rA | 1 |
| | mAmCrCmGrAmUrUmUrCmArGmArUmGrGmUrGmCrUmAG | 5 |
| Teno43 | /5Phos/fUfAfGfCfAfCfCfAfUfCfUfGfAfAfAfUfCfGfGfUmUmA | 1 |
| | mAmCrCmGrAmUrUmUrCmArGmArUmGrGmUrGmCrUA | 6 |
| Teno44 | /5Phos/fUfAfGfCfAfCfCfAfUfCfUfGfAfAfAfUfCfGfGfUmUmA | 1 |
| | mAmCrCmGrAmUrUmUrCmArGmArUmGrGmUrGmCrUmAmG | 5 |
| Teno45 | /5Phos/fUfAfGfCfAfCfCfAfUfCfUfGfAfAfAfUfCfGfGfUmUmA | 1 |
| | mAmCrCmGrAmUrUmUrCmArGmArUmGrGmUrGmCrUmAG | 5 |
| Teno46 | /5Phos/rUrArGrCrArCrCrArUrCrUrGrArArArUrCrGrGrUrUrA | 1 |
| (native) | rArCrUrGrArUrUrUrCrUrUrUrUrGrGrUrGrUrUrCrArG | 2 |
| Teno47 | /5Phos/fUrArGfCrAfCfCrAfUfCfUrGrArArAfUfCrGrGfUfUmA | 1 |
| | mAmCrCmGrAmUrUmUrCmArGmArUmGrGmUrGmCrUA | 6 |
| Teno48 | /5Phos/fUrArGfCrAfCfCrAfUfCfUrGrArArAfUfCrGrGfUfUmA | 1 |
| | mAmCrCmGrAmUrUmUrCmArGmArUmGrGmUrGmCrUmAmG | 5 |
| Teno49 | /5Phos/fUrArGfCrAfCfCrAfUfCfUrGrArArAfUfCrGrGfUfUmA | 1 |
| | mAmCrCmGrAmUrUmUrCmArGmArUmGrGmUrGmCrUmAG | 5 |

/5Phos/ = 5′ phosphate
mA = 2′-O-methyl adenosine ribonucleoside;
mC = 2′-O-methyl cytosine ribonucleoside;
mG = 2′-O-methyl guanine ribonucleoside;
mU = 2′-O-methyl uracil ribonucleoside;
rA = adenosine ribonucleoside;
rC = cytosine ribonucleoside;
rG = guanine ribonucleoside;
rU = uracil ribonucleoside;
fA = 2′-deoxy-fluoro adenosine ribonucleoside;
fC = 2′-deoxy-fluoro cytosine ribonucleoside;
fG = 2′-deoxy-fluoro guanine ribonucleoside;
fU = 2′-deoxy-fluoro uracil ribonucleoside;
*rA = 5′-O-phosphorothioate adenosine ribonucleoside;
*rC = 5′-O-phosphorothioate cytosine ribonucleoside;
*rG = 5′-O-phosphorothioate guanine ribonucleoside;
*rU = 5′-O-phosphorothioate uracil ribonucleoside;
A = adenosine deoxyribonucleoside;
C = cytosine deoxyribonucleoside;
G = guanine deoxyribonucleoside;
U = uracil deoxyribonucleoside.

Thus the following passenger strand nucleobase sequences were tested, see Table 1b. SEQ ID NO: 2 represents the native human miR-29a passenger strand sequence. SEQ ID NO: 3 differs from SEQ ID NO: 2 at one position, as shown in underlining. SEQ ID NO: 4 differs from SEQ ID NO: 2 at five positions. SEQ ID NO: 5 differs from SEQ ID NO: 2 at six positions. SEQ ID NO: 6 differs from SEQ ID NO: 2 at seven positions. SEQ ID NO: 7 is the reverse sequence of SEQ ID NO: 2 and represents a negative control.

TABLE 1b
Passenger strand nucleobase sequences

| Sequence | SEQ ID NO |
|---|---|
| ACUGAUUUCUUUUGGUGUUCAG | 2 |
| ACUGAUUUCUUUUGGUGUUCA- | 3 |
| ACCGAUUUCAAUUGGUGCU-AG | 4 |
| ACCGAUUUCAGAUGGUGCU-AG | 5 |
| ACCGAUUUCAGAUGGUGCU-A- | 6 |
| GACUUGUGGUUUUCUUUAGUCA | 7 |
| (reverse of SEQ ID NO: 2) | |

Table 1c below shows the combination of 2′ sugar modifications, nucleobase sequence (as SEQ ID NOs), and the internucleoside linkages in each compound. Teno1-Teno49 contain one of five types of guide strand (indicated as G1-G5 below), and one of 11 types of passenger strand (indicated as P1-P11 below). Teno36 is an exception as it does not contain a passenger strand. All sequences are shown in the 5′ to 3′ direction.

Taking Teno1 as an example, Table 1c shows that it contains the “G1” guide strand, which has the nucleobase sequence of SEQ ID NO: 1, wherein three nucleosides at the 3′ end are 2′-O-methyl ribonucleosides and the remaining nucleosides are unmodified ribonucleosides, and all internucleoside linkages are phosphodiester. The passenger strand is “P1”, having the nucleobase sequence of SEQ ID NO: 2 and wherein all nucleosides are alternately 2′-O-methyl and unmodified ribonucleosides, and all internucleoside linkages are phosphodiester. Teno2 contains the same guide strand as Teno1 (“G1”), but a different passenger strand (“P2”), etc.

TABLE 1c
MiR-29 compounds-grouped by guide strand

| | | Nucleo- | | | |
|---|---|---|---|---|---|
| | | base | | | |
| Compound | Modifications | SEQ ID NO | Sugars | Linkages | # |
| Teno1 | rrrrrrrrrrrrrrrrrrrmmm | 1 | rₙm₃ | oₙ | G1 |
| | mrmrmrmrmrmrnrmrmrmrmr | 2 | (mr)ₙ | oₙ | P1 |
| Teno2 | rrrrrrrrrrrrrrrrrrrmmm | 1 | rₙm₃ | oₙ | G1 |
| | mrmrmrmrmrmrmrmrmrmrm | 4 | (mr)ₙm | oₙ | P2 |
| Teno3 | rrrrrrrrrrrrrrrrrrrmmm | 1 | rₙm₃ | oₙ | G1 |
| | rrrrrrrrrrrrrrrrrrrrrr | 7 | rₙ | oₙ | P3 |
| Teno4 | rrrrrrrrrrrrrrrrrrrmmm | 1 | rₙm₃ | oₙ | G1 |
| | mrmrmrmrmrmrmrmrmrmrd | 3 | (mr)ₙd | oₙ | P4 |
| Teno5 | rrrrrrrrrrrrrrrrrrrmmm | 1 | rₙm₃ | oₙ | G1 |
| | mrmrmrmrmrmrmrmrmrmrmd | 2 | (mr)ₙmd | oₙ | P5 |
| Teno6 | rrrrrrrrrrrrrrrrrrrmmm | 1 | rₙm₃ | oₙ | G1 |
| | mmrmrmrmrmrmrmrmrmrmmm | 2 | m(mr)ₙm₃ | oₙ | P6 |
| Teno7 | rrrrrrrrrrrrrrrrrrrmmm | 1 | rₙm₃ | oₙ | G1 |
| | mmrmrmrmrmrmrmrmrmrmm | 4 | m(mr)ₙm₂ | oₙ | P7 |
| Teno37 | rrrrrrrrrrrrrrrrrrrmmm | 1 | rₙm₃ | oₙ | G1 |
| | mmrmrmrmrmrmrmrmrmrd | 6 | m(mr)d | oₙ | P8 |
| Teno38 | rrrrrrrrrrrrrrrrrrrmmm | 1 | rₙm₃ | oₙ | G1 |
| | mmrmrmrmrmrmrmrmrmrmm | 5 | m(mr)ₙm₂ | oₙ | P9 |
| Teno39 | rrrrrrrrrrrrrrrrrrrmmm | 1 | rₙm₃ | oₙ | G1 |
| | mmrmrmrmrmrmrmrmrmrmd | 5 | m(mr)ₙmd | oₙ | P10 |
| Teno8 | rrrrrrrrrrrrrrrrrrrr*r*r | 1 | rₙ | oₙs₂ | G2 |
| | mrmrmrmrmrmrmrmrmrmrmr | 2 | (mr)ₙ | oₙ | P1 |
| Teno9 | rrrrrrrrrrrrrrrrrrrr*r*r | 1 | rₙ | oₙs₂ | G2 |
| | mrmrmrmrmrmrmrmrmrmrm | 4 | (mr)ₙm | oₙ | P2 |
| Teno10 | rrrrrrrrrrrrrrrrrrrr*r*r | 1 | rₙ | oₙs₂ | G2 |
| | rrrrrrrrrrrrrrrrrrrrrr | 7 | rₙ | oₙ | P3 |
| Teno11 | rrrrrrrrrrrrrrrrrrrr*r*r | 1 | rₙ | oₙs₂ | G2 |
| | mrmrmrmrmrmrmrmrmrmrd | 3 | (mr)ₙd | oₙ | P4 |
| Teno12 | rrrrrrrrrrrrrrrrrrrr*r*r | 1 | rₙ | oₙs₂ | G2 |
| | mrmrmrmrmrmrmrmrmrmrmd | 2 | (mr)ₙmd | oₙ | P5 |
| Teno13 | rrrrrrrrrrrrrrrrrrrr*r*r | 1 | rₙ | oₙs₂ | G2 |
| | mmrmrmrmrmrmrmrmrmrmmm | 2 | m(mr)ₙm₃ | oₙ | P6 |
| Teno14 | rrrrrrrrrrrrrrrrrrrr*r*r | 1 | rₙ | oₙs₂ | G2 |
| | mmrmrmrmrmrmrmrmrmrmm | 4 | m(mr)ₙm₂ | oₙ | P7 |
| Teno40 | rrrrrrrrrrrrrrrrrrrr*r*r | 1 | rₙ | oₙs₂ | G2 |
| | mmrmrmrmrmrmrmrmrmrd | 6 | m(mr)d | oₙ | P8 |
| Teno41 | rrrrrrrrrrrrrrrrrrrr*r*r | 1 | rₙ | oₙs₂ | G2 |
| | mmrmrmrmrmrmrmrmrmrmm | 5 | m(mr)ₙm₂ | oₙ | P9 |
| Teno42 | rrrrrrrrrrrrrrrrrrrr*r*r | 1 | rₙ | oₙs₂ | G2 |
| | mmrmrmrmrmrmrmrmrmrmd | 5 | m(mr)ₙmd | oₙ | P10 |
| Teno15 | ffffffffffffffffffffmm | 1 | rₙm₂ | oₙ | G3 |
| | mrmrmrmrmrmrmrmrmrmrmr | 2 | (mr)ₙ | oₙ | P1 |
| Teno16 | ffffffffffffffffffffmm | 1 | fₙm₂ | oₙ | G3 |
| | mrmrmrmrmrmrmrmrmrmrm | 4 | (mr)ₙm | oₙ | P2 |
| Teno17 | ffffffffffffffffffffmm | 1 | fₙm₂ | oₙ | G3 |
| | rrrrrrrrrrrrrrrrrrrrrr | 7 | rₙ | oₙ | P3 |
| Teno18 | ffffffffffffffffffffmm | 1 | fₙm₂ | oₙ | G3 |
| | mrmrmrmrmrmrmrmrmrmrd | 3 | (mr)ₙd | oₙ | P4 |
| Teno19 | ffffffffffffffffffffmm | 1 | fₙm₂ | oₙ | G3 |
| | mrmrmrmrmrmrmrmrmrmrmd | 2 | (mr)ₙmd | oₙ | P5 |
| Teno20 | ffffffffffffffffffffmm | 1 | fₙm₂ | oₙ | G3 |
| | mmrmrmrmrmrmrmrmrmrmmm | 2 | m(mr)ₙm₃ | oₙ | P6 |
| Teno21 | ffffffffffffffffffffmm | 1 | fₙm₂ | oₙ | G3 |
| | mmrmrmrmrmrmrmrmrmrmm | 4 | m(mr)ₙm₂ | oₙ | P7 |
| Teno43 | ffffffffffffffffffffmm | 1 | fₙm₂ | oₙ | G3 |
| | mmrmrmrmrmrmrmrmrmrd | 6 | m(mr)d | oₙ | P8 |
| Teno44 | ffffffffffffffffffffmm | 1 | fₙm₂ | oₙ | G3 |
| | mmrmrmrmrmrmrmrmrmrmm | 5 | m(mr)ₙm₂ | oₙ | P9 |
| Teno45 | ffffffffffffffffffffmm | 1 | fₙm₂ | oₙ | G3 |
| | mmrmrmrmrmrmrmrmrmrmd | 5 | m(mr)ₙmd | oₙ | P10 |
| Teno36 | ffffffffffffffffffffmm | 1 | fₙm₂ | oₙ | G3 |
| | No passenger strand | — | — | — | — |
| Teno22 | rrrrrrrrrrrrrrrrrrrrrr | 1 | rₙ | oₙ | G4 |
| | mrmrmrmrmrmrmrmrmrmrmr | 2 | (mr)ₙ | oₙ | P1 |
| Teno23 | rrrrrrrrrrrrrrrrrrrrrr | 1 | rₙ | oₙ | G4 |
| | mrmrmrmrmrmrmrmrmrmrm | 4 | (mr)ₙm | oₙ | P2 |
| Teno24 | rrrrrrrrrrrrrrrrrrrrrr | 1 | rₙ | oₙ | G4 |
| | rrrrrrrrrrrrrrrrrrrrrr | 7 | rₙ | oₙ | P3 |
| Teno25 | rrrrrrrrrrrrrrrrrrrrrr | 1 | rₙ | oₙ | G4 |
| | mrmrmrmrmrmrmrmrmrmrd | 3 | (mr)ₙd | oₙ | P4 |
| Teno26 | rrrrrrrrrrrrrrrrrrrrrr | 1 | rₙ | oₙ | G4 |
| | mrmrmrmrmrmrmrmrmrmrmd | 2 | (mr)ₙmd | oₙ | P5 |
| Teno27 | rrrrrrrrrrrrrrrrrrrrrr | 1 | rₙ | oₙ | G4 |
| | mmrmrmrmrmrmrmrmrmrmmm | 2 | m(mr)ₙm₃ | oₙ | P6 |
| Teno28 | rrrrrrrrrrrrrrrrrrrrrr | 1 | rₙ | oₙ | G4 |
| | mmrmrmrmrmrmrmrmrmrmm | 4 | m(mr)ₙm₂ | oₙ | P7 |
| Teno46 | rrrrrrrrrrrrrrrrrrrrrr | 1 | rₙ | oₙ | G4 |
| (native) | rrrrrrrrrrrrrrrrrrrrrr | 2 | rₙ | oₙ | P11 |
| Teno29 | frrfrffrfffrrrrffrrffm | 1 | (f, r)ₙm | oₙ | G5 |
| | mrmrmrmrmrmrmrmrmrmrmr | 2 | (mr)ₙ | oₙ | P1 |
| Teno30 | frrfrffrfffrrrrffrrffm | 1 | (f, r)ₙm | oₙ | G5 |
| | mrmrmrmrmrmrmrmrmrmrm | 4 | (mr)ₙm | oₙ | P2 |
| Teno31 | frrfrffrfffrrrrffrrffm | 1 | (f, r)ₙm | oₙ | G5 |
| | rrrrrrrrrrrrrrrrrrrrrr | 7 | rₙ | oₙ | P3 |
| Teno32 | frrfrffrfffrrrrffrrffm | 1 | (f, r)ₙm | oₙ | G5 |
| | mrmrmrmrmrmrmrmrmrmrd | 3 | (mr)ₙd | oₙ | P4 |
| Teno33 | frrfrffrfffrrrrffrrffm | 1 | (f, r)ₙm | oₙ | G5 |
| | mrmrmrmrmrmrmrmrmrmrmd | 2 | (mr)ₙmd | oₙ | P5 |
| Teno34 | frrfrffrfffrrrrffrrffm | 1 | (f, r)ₙm | oₙ | G5 |
| | mmrmrmrmrmrmrmrmrmrmmm | 2 | m(mr)ₙm₃ | oₙ | P6 |
| Teno35 | frrfrffrfffrrrrffrrffm | 1 | (f, r)ₙm | oₙ | G5 |
| | mmrmrmrmrmrmrmrmrmrmm | 4 | m(mr)ₙm₂ | oₙ | P7 |
| Teno47 | frrfrffrfffrrrrffrrffm | 1 | (f, r)ₙm | oₙ | G5 |
| | mmrmrmrmrmrmrmrmrmrd | 6 | m(mr)d | oₙ | P8 |
| Teno48 | frrfrffrfffrrrrffrrffm | 1 | (f, r)ₙm | oₙ | G5 |
| | mmrmrmrmrmrmrmrmrmrmm | 5 | m(mr)ₙm₂ | oₙ | P9 |
| Teno49 | frrfrffrfffrrrrffrrffm | 1 | (f, r)ₙm | oₙ | G5 |
| | mmrmrmrmrmrmrmrmrmrmd | 5 | m(mr)ₙmd | oₙ | P10 |

m = 2′-O-methyl ribonucleotide (-O-Me);
f = 2′-deoxy-fluoro ribonucleotide (-F);
d = 2′-deoxyribonucleotide (-H);
r = unmodified ribonucleotide (-OH);
o = 5′-O-phosphodiester internucleoside linkage;
* or s = 5′-O-phosphorothioate internucleoside linkage;
n = n number of nucleosides.

Table 1d groups the compounds according to their passenger strand.

TABLE 1d
MIR-29 compounds - grouped by passenger strand

| | | Nucleo- | | | |
|---|---|---|---|---|---|
| | | base | | | |
| Compound | Modifications | SEQ ID NO | Sugars | Linkages | # |
| Teno1 | rrrrrrrrrrrrrrrrrrrmmm | 1 | rₙm₃ | oₙ | G1 |
| | mrmrmrmrmrmrmrmrmrmrmr | 2 | (mr)ₙ | oₙ | P1 |
| Teno8 | rrrrrrrrrrrrrrrrrrrr*r*r | 1 | rₙ | oₙs₂ | G2 |
| | mrmrmrmrmrmrmrmrmrmrmr | 2 | (mr)ₙ | oₙ | P1 |
| Teno15 | ffffffffffffffffffffmm | 1 | fₙm₂ | oₙ | G3 |
| | mrmrmrmrmrmrmrmrmrmrmr | 2 | (mr)ₙ | oₙ | P1 |
| Teno22 | rrrrrrrrrrrrrrrrrrrrrr | 1 | rₙ | oₙ | G4 |
| | mrmrmrmrmrmrmrmrmrmrmr | 2 | (mr)ₙ | oₙ | P1 |
| Teno29 | frrfrffrfffrrrrffrrffm | 1 | (f, r)ₙm | oₙ | G5 |
| | mrmrmrmrmrmrmrmrmrmrmr | 2 | (mr)ₙ | oₙ | P1 |
| Teno2 | rrrrrrrrrrrrrrrrrrrmmm | 1 | rₙm₃ | oₙ | G1 |
| | mrmrmrmrmrmrmrmrmrmrm | 4 | (mr)ₙm | oₙ | P2 |
| Teno9 | rrrrrrrrrrrrrrrrrrrr*r*r | 1 | rₙ | oₙs₂ | G2 |
| | mrmrmrmrmrmrmrmrmrmrm | 4 | (mr)ₙm | oₙ | P2 |
| Teno16 | ffffffffffffffffffffmm | 1 | fₙm₂ | oₙ | G3 |
| | mrmrmrmrmrmrmrmrmrmrm | 4 | (mr)ₙm | oₙ | P2 |
| Teno23 | rrrrrrrrrrrrrrrrrrrrrr | 1 | rₙ | oₙ | G4 |
| | mrmrmrmrmrmrmrmrmrmrm | 4 | (mr)ₙm | oₙ | P2 |
| Teno30 | frrfrffrfffrrrrffrrffm | 1 | (f, r)ₙm | oₙ | G5 |
| | mrmrmrmrmrmrmrmrmrmrm | 4 | (mr)ₙm | oₙ | P2 |
| Teno3 | rrrrrrrrrrrrrrrrrrrmmm | 1 | rₙm₃ | oₙ | G1 |
| | rrrrrrrrrrrrrrrrrrrrrr | 7 | rₙ | oₙ | P3 |
| Teno10 | rrrrrrrrrrrrrrrrrrrr*r*r | 1 | rₙ | oₙs₂ | G2 |
| | rrrrrrrrrrrrrrrrrrrrrr | 7 | rₙ | oₙ | P3 |
| Teno17 | ffffffffffffffffffffmm | 1 | fₙm₂ | oₙ | G3 |
| | rrrrrrrrrrrrrrrrrrrrrr | 7 | rₙ | oₙ | P3 |
| Teno24 | rrrrrrrrrrrrrrrrrrrrrr | 1 | rₙ | oₙ | G4 |
| | rrrrrrrrrrrrrrrrrrrrrr | 7 | rₙ | oₙ | P3 |
| Teno31 | frrfrffrfffrrrrffrrffm | 1 | (f, r)ₙm | oₙ | G5 |
| | rrrrrrrrrrrrrrrrrrrrrr | 7 | rₙ | oₙ | P3 |
| Teno4 | rrrrrrrrrrrrrrrrrrrmmm | 1 | rₙm₃ | oₙ | G1 |
| | mrmrmrmrmrmrmrmrmrmrd | 3 | (mr)ₙd | oₙ | P4 |
| Teno11 | rrrrrrrrrrrrrrrrrrrr*r*r | 1 | rₙ | oₙs₂ | G2 |
| | mrmrmrmrmrmrmrmrmrmrd | 3 | (mr)ₙd | oₙ | P4 |
| Teno18 | ffffffffffffffffffffmm | 1 | fₙm₂ | oₙ | G3 |
| | mrmrmrmrmrmrmrmrmrmrd | 3 | (mr)ₙd | oₙ | P4 |
| Teno25 | rrrrrrrrrrrrrrrrrrrrrr | 1 | rₙ | oₙ | G4 |
| | mrmrmrmrmrmrmrmrmrmrd | 3 | (mr)ₙd | oₙ | P4 |
| Teno32 | frrfrffrfffrrrrffrrffm | 1 | (f, r)ₙm | oₙ | G5 |
| | mrmrmrmrmrmrmrmrmrmrd | 3 | (mr)ₙd | oₙ | P4 |
| Teno5 | rrrrrrrrrrrrrrrrrrrmmm | 1 | rₙm₃ | oₙ | G1 |
| | mrmrmrmrmrmrmrmrmrmrmd | 2 | (mr)ₙmd | oₙ | P5 |
| Teno12 | rrrrrrrrrrrrrrrrrrrr*r*r | 1 | rₙ | oₙs₂ | G2 |
| | mrmrmrmrmrmrmrmrmrmrmd | 2 | (mr)ₙmd | oₙ | P5 |
| Teno19 | ffffffffffffffffffffmm | 1 | fₙm₂ | oₙ | G3 |
| | mrmrmrmrmrmrmrmrmrmrmd | 2 | (mr)ₙmd | oₙ | P5 |
| Teno26 | rrrrrrrrrrrrrrrrrrrrrr | 1 | rₙ | oₙ | G4 |
| | mrmrmrmrmrmrmrmrmrmrmd | 2 | (mr)ₙmd | oₙ | P5 |
| Teno33 | frrfrffrfffrrrrffrrffm | 1 | (f, r)ₙm | oₙ | G5 |
| | mrmrmrmrmrmrmrmrmrmrmd | 2 | (mr)ₙmd | oₙ | P5 |
| Teno6 | rrrrrrrrrrrrrrrrrrrmmm | 1 | rₙm₃ | oₙ | G1 |
| | mmrmrmrmrmrmrmrmrmrmmm | 2 | m(mr)ₙm₃ | oₙ | P6 |
| Teno13 | rrrrrrrrrrrrrrrrrrrr*r*r | 1 | rₙ | oₙs₂ | G2 |
| | mmrmrmrmrmrmrmrmrmrmmm | 2 | m(mr)ₙm₃ | oₙ | P6 |
| Teno20 | ffffffffffffffffffffmm | 1 | fₙm₂ | oₙ | G3 |
| | mmrmrmrmrmrmrmrmrmrmmm | 2 | m(mr)ₙm₃ | oₙ | P6 |
| Teno27 | rrrrrrrrrrrrrrrrrrrrrr | 1 | rₙ | oₙ | G4 |
| | mmrmrmrmrmrmrmrmrmrmmm | 2 | m(mr)ₙm₃ | oₙ | P6 |
| Teno34 | frrfrffrfffrrrrffrrffm | 1 | (f, r)ₙm | oₙ | G5 |
| | mmrmrmrmrmrmrmrmrmrmmm | 2 | m(mr)ₙm₃ | oₙ | P6 |
| Teno7 | rrrrrrrrrrrrrrrrrrrmmm | 1 | rₙm₃ | oₙ | G1 |
| | mmrmrmrmrmrmrmrmrmrmm | 4 | m(mr)ₙm₂ | oₙ | P7 |
| Teno14 | rrrrrrrrrrrrrrrrrrrr*r*r | 1 | rₙ | oₙs₂ | G2 |
| | mmrmrmrmrmrmrmrmrmrmm | 4 | m(mr)ₙm₂ | oₙ | P7 |
| Teno21 | ffffffffffffffffffffmm | 1 | fₙm₂ | oₙ | G3 |
| | mmrmrmrmrmrmrmrmrmrmm | 4 | m(mr)ₙm₂ | oₙ | P7 |
| Teno28 | rrrrrrrrrrrrrrrrrrrrrr | 1 | rₙ | oₙ | G4 |
| | mmrmrmrmrmrmrmrmrmrmm | 4 | m(mr)ₙm₂ | oₙ | P7 |
| Teno35 | frrfrffrfffrrrrffrrffm | 1 | (f, r)ₙm | oₙ | G5 |
| | mmrmrmrmrmrmrmrmrmrmm | 4 | m(mr)ₙm₂ | oₙ | P7 |
| Teno37 | rrrrrrrrrrrrrrrrrrrmmm | 1 | rₙm₃ | oₙ | G1 |
| | mmrmrmrmrmrmrmrmrmrd | 6 | m(mr)d | oₙ | P8 |
| Teno40 | rrrrrrrrrrrrrrrrrrrr*r*r | 1 | rₙ | oₙs₂ | G2 |
| | mmrmrmrmrmrmrmrmrmrd | 6 | m(mr)d | oₙ | P8 |
| Teno43 | ffffffffffffffffffffmm | 1 | fₙm₂ | oₙ | G3 |
| | mmrmrmrmrmrmrmrmrmrd | 6 | m(mr)d | oₙ | P8 |
| Teno47 | frrfrffrfffrrrrffrrffm | 1 | (f, r)ₙm | oₙ | G5 |
| | mmrmrmrmrmrmrmrmrmrd | 6 | m(mr)d | oₙ | P8 |
| Teno38 | rrrrrrrrrrrrrrrrrrrmmm | 1 | rₙm₃ | oₙ | G1 |
| | mmrmrmrmrmrmrmrmrmrmm | 5 | m(mr)ₙm₂ | oₙ | P9 |
| Teno41 | rrrrrrrrrrrrrrrrrrrr*r*r | 1 | rₙ | oₙs₂ | G2 |
| | mmrmrmrmrmrmrmrmrmrmm | 5 | m(mr)nm₂ | oₙ | P9 |
| Teno44 | ffffffffffffffffffffmm | 1 | fₙm₂ | oₙ | G3 |
| | mmrmrmrmrmrmrmrmrmrmm | 5 | m(mr)nm₂ | oₙ | P9 |
| Teno48 | frrfrffrfffrrrrffrrffm | 1 | (f, r)ₙm | oₙ | G5 |
| | mmrmrmrmrmrmrmrmrmrmm | 5 | m(mr)ₙm₂ | oₙ | P9 |
| Teno39 | rrrrrrrrrrrrrrrrrrrmmm | 1 | rₙm₃ | oₙ | G1 |
| | mmrmrmrmrmrmrmrmrmrmd | 5 | m(mr)ₙmd | oₙ | P10 |
| Teno42 | rrrrrrrrrrrrrrrrrrrr*r*r | 1 | rₙ | oₙs₂ | G2 |
| | mmrmrmrmrmrmrmrmrmrmd | 5 | m(mr)ₙmd | oₙ | P10 |
| Teno45 | ffffffffffffffffffffmm | 1 | fₙm₂ | oₙ | G3 |
| | mmrmrmrmrmrmrmrmrmrmd | 5 | m(mr)ₙmd | oₙ | P10 |
| Teno49 | frrfrffrfffrrrrffrrffm | 1 | (f, r)ₙm | oₙ | G5 |
| | mmrmrmrmrmrmrmrmrmrmd | 5 | m(mr)ₙmd | oₙ | P10 |
| Teno36 | ffffffffffffffffffffmm | 1 | fₙm₂ | oₙ | G3 |
| | No passenger strand | — | — | — | — |
| Teno46 | rrrrrrrrrrrrrrrrrrrrrr | 1 | rₙ | oₙ | G4 |
| (native) | rrrrrrrrrrrrrrrrrrrrrr | 2 | rₙ | oₙ | P11 |

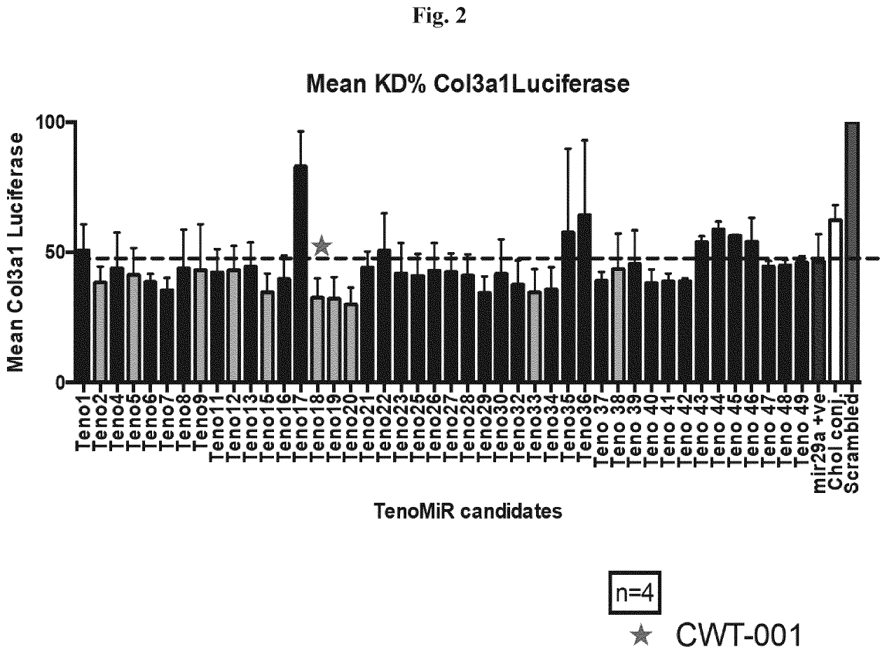

The compounds were tested for activity relative to native human mature miR29a using an in vitro collagen 3 luciferase reporter assay. The basis of this assay is a luciferase reporter gene containing the miR29a binding sites from collagen 3. In this assay luciferase activity is inversely proportional to miR29a activity. This means that high miR29a activity more strongly suppresses collagen 3 expression, which results in a lower luciferase signal. The luciferase assays were performed in HEK293 cells, after transfection of the miR-29 compounds with transfection agent. Luciferase activity of the miR-29 compounds is compared to cells treated with a commercially available human native miR29a positive control (“mir29a+ve”) and scrambled miR29a negative control (“scrambled”) (n=4). Results are shown in FIG. 2.

The reference to “Chol. conj.” in FIG. 2 refers to the commercially available miR29a positive control which was conjugated to a cholesterol moiety at the 3′ end of the passenger strand, and added to the HEK293 cells without the use of any transfection agent. FIG. 2 indicates that the cholesterol-conjugated compound is able to enter the cells and suppress collagen 3 expression. The higher luciferase signal with the cholesterol-conjugated compound as compared to unconjugated miR29a positive control is thought to reflect the effect of transfection agent which was used for the transfection of unconjugated miR29a positive control, whereas transfection agent was absent when cholesterol-conjugated compound was transfected.

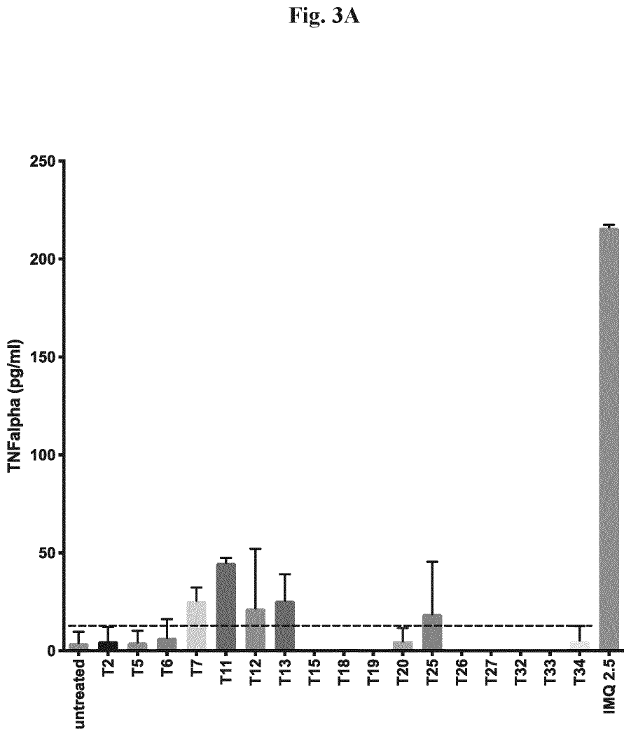

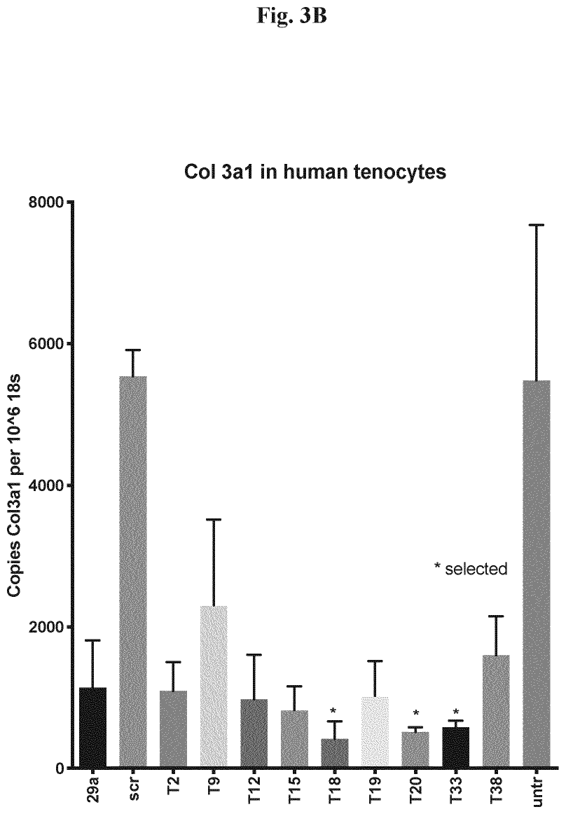

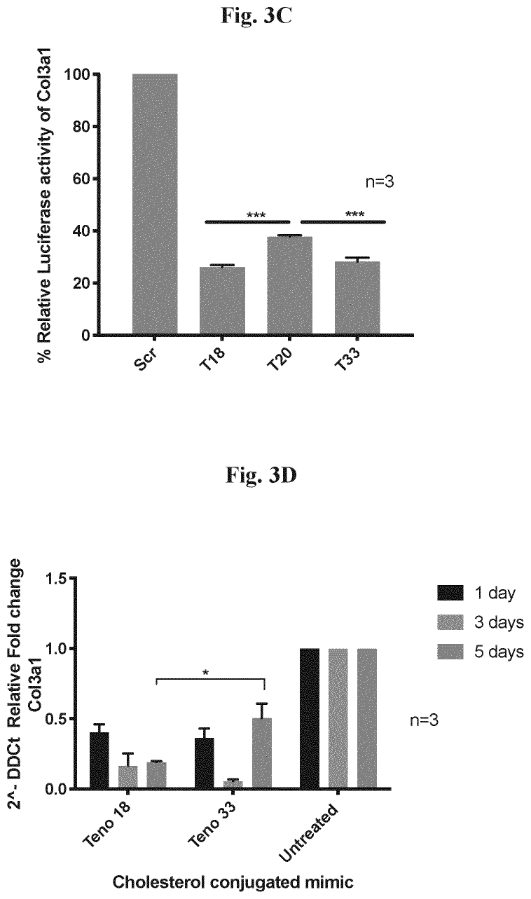

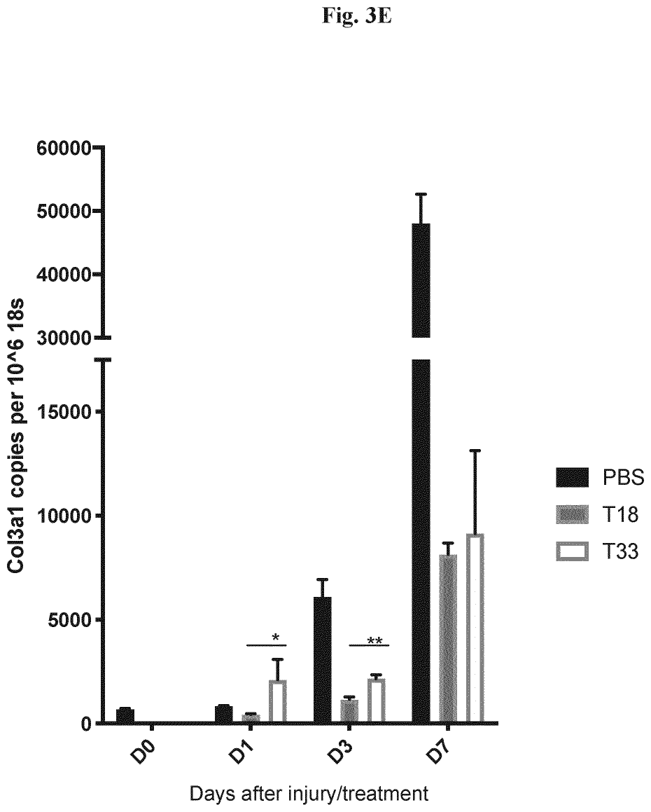

A selection of compounds (Teno2, Teno5, Teno6, Teno7, Teno1l, Teno12, Teno13, Teno15, Teno18, Teno19, Teno20, Teno25, Teno26, Teno27, Teno32, Teno33 and Teno34) was tested for potential immunogenicity by testing their capacity to induce the production of pro-inflammatory cytokine TNF-alpha in primary human macrophages. Cultured primary human macrophages, which as key components of the innate immune system are sensitive to ds-RNA molecules, were treated with 0.35 pg/ml miR-29 compound for 24 hrs (n=4) and the levels of the pro-inflammatory cytokine tumour necrosis factor-alpha (TNFα) measured by ELISA. Imiquimod (IMQ), a TLR7/8 agonist, was used as a positive control (2.5 μg/ml). Results are shown in FIG. 3A. Compounds that induced the expression of more than 10 pg/ml TNF-alpha were not taken forward. However, Teno12 was taken forward as it represented a miR-29 compound with the lowest immunogenicity amongst those tested that contained a guide strand with phosphorothioate internucleoside linkages (Teno1l, Teno12 and Teno13).

A selection of compounds (Teno2, Teno9, Teno12, Teno15, Teno18, Teno19, Teno20, Teno33 and Teno38) was therefore tested for the ability to inhibit the expression of col3a1 in human tenocytes. The results are shown in FIG. 3B. This led to the selection of three miR-29 compounds: Teno18, Teno20 and Teno33.

| | | Nucleo- |
|---|---|---|
| | | base |
| Compound | Formula | SEQ ID NO |
| Teno18 | /5Phos/fUfAfGfCfAfCfCfAfUfCfUfGfAfAfAfUfCfGfGfUmUmA | 1 |
| | mArCmUrGmArUmUrUmCrUmUrUmUrGmGrUmGrUmUrCA | 3 |
| Teno20 | /5Phos/fUfAfGfCfAfCfCfAfUfCfUfGfAfAfAfUfCfGfGfUmUmA | 1 |
| | mAmCrUmGrAmUrUmUrCmUrUmUrUmGrGmUrGmUrUmCmAmG | 2 |
| Teno33 | /5Phos/fUrArGfCrAfCfCrAfUfCfUrGrArArAfUfCrGrGfUfUmA | 1 |
| | mArCmUrGmArUmUrUmCrUmUrUmUrGmGrUmGrUmUrCmAG | 2 |

The stability of Teno18, Teno20 and Teno33 was confirmed by measuring their activity in the in vitro collagen 3 luciferase reporter assay after storage at 37° C. for three months. All compounds were stable. Teno18 and Teno33 showed the highest activity after storage, followed by Teno20 (FIG. 3C).

Based on the above data, Teno18 and Teno33 were taken forward for in vivo activity testing in human tenocytes and a mouse tendon injury model. To enhance cellular uptake in vivo, a cholesterol moiety was conjugated to the 3′ end of the passenger strand of each compound (e.g., see FIG. 1B for Teno18). Both Teno18 and Teno33 effectively knocked down col3a1 expression in human tenocytes (FIG. 3D) and in a mouse tendon injury model (FIG. 3E). Treatment with Teno18 knocked down col3a1 expression most effectively at the day 5 time point in human tenocytes (FIG. 3D), and at the 1 day and 3 day time points in the mouse tendon injury model (FIG. 3E).

Teno18 was therefore selected as the clinical candidate for further in vivo testing, described in the Examples that follow.

The drug substance (cholesterol-conjugated Teno18, designated CWT-001) is synthesised using routine methods (solid phase synthesis) and provided as a lyophilised powder. The drug product can be provided as a sterile solution containing 5 mg/mL CWT-001, typically at pH 7.3, in clear glass vials, which is diluted with saline for injection prior to administration. Alternatively, CWT-001 can be provided as a sterile solution at the final concentration for administration (e.g., about 200 μg/ml, 500 μg/ml, 1500 μg/ml, 2000 μg/ml, 4500 μg/ml or 5000 μg/ml, such as about 200 μg/ml, 500 μg/ml, 1500 μg/ml, or 4500 μg/ml). In order to optimise the stability of the drug substance, EDTA can be added, e.g., at a final concentration of 0.001%-0.005%, e.g., about 0.003% (100 PM).

The drug product vials can be stored and transported at −20° C. and are intended for single use. Frozen vials appear cloudy white. Upon thawing to room temperature, the active pharmaceutical ingredient (API) will solubilise and become a clear colourless solution.

The structure of the CWT-001 drug substance (as a sodium salt) is shown in FIG. 1. FIG. 1A shows the guide strand, and FIG. 1B shows the passenger strand, conjugated to a cholesterol moiety at the 3′ end.

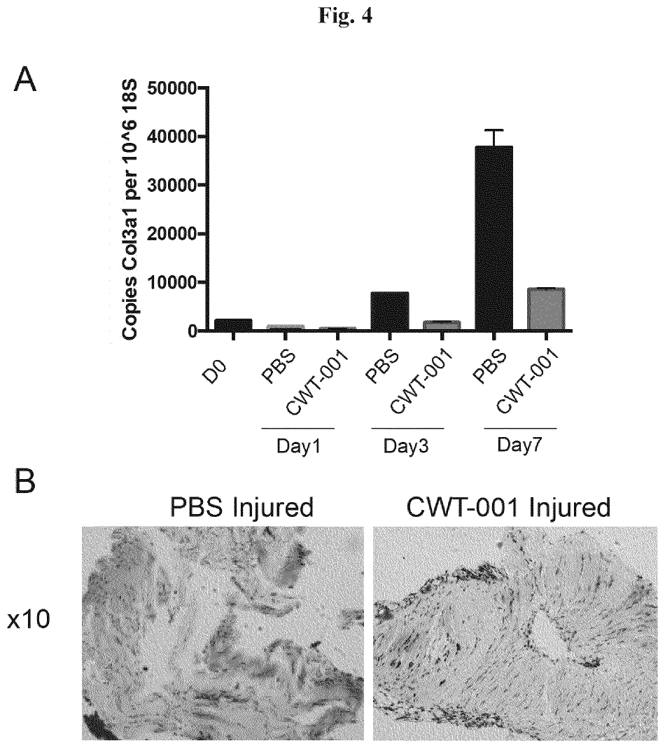

### Example 2: In Vivo Primary Pharmacology Studies (FIG. 4)

As rodents have similar limb anatomy as humans, including tendons that are prone to tendinopathy (Achilles, patellar and supraspinatus tendons), a validated mouse tendon injury model (ref. 12) was selected to test if treatment with CWT-001 significantly reduced collagen 3 expression and improved tendon healing. In these experiments, tendon injury was induced using a 0.75 mm diameter biopsy needle followed immediately by injection of 0.35 μg/ml CWT-001 into the site of injury. Mice were allowed to recover for 1, 3 or 7 days and tendons removed for histological and molecular analysis.

Treatment with CWT-001 significantly improved tendon fibre alignment and collagen production via the suppression of collagen 3 expression in tendon disease (FIGS. 4A and 4B).

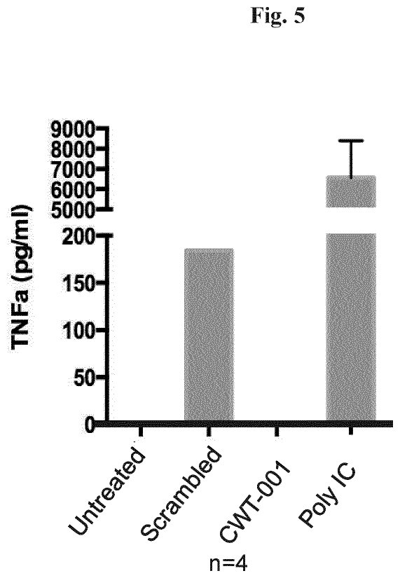

### Example 3: Secondary Pharmacology (FIGS. 5-7)

### Example 3a—Bioinformatics Analysis of CWT-001's Potential Off-Target Effects

MicroRNAs by their nature interact with large numbers of target mRNA species in most cases via base-pairing of a 6-8 nucleotide seed sequence. The sequence of CWT-001's guide strand is identical to that of natural miR29a meaning that CWT-001 will target the same mRNAs as the natural molecule via a RISC-dependent mechanism. To address the possibility of potential hybridisation-dependent off-target effects, a bioinformatics analysis of both guide and passenger strands was conducted as recommended by the Oligonucleotide Safety Working Group guidance (see ref. 13). This analysis generated a list of 52 genes detailed below, which could theoretically hybridise with CWT-001's guide and passenger strands. Mutations in 13 of these genes were associated with known human diseases, as also detailed below.

A1) In Silico Evaluation of Potential Hybridization-Dependent Liabilities for the CWT-001 Guide Strand in Precursor microRNA Sequences

Q-PCR analysis was performed on the expression levels of miRNAs in primary human tenocytes after treatment with CWT-001. Potential cross-hybridization targets were identified as hsa-miR29a, hsa-miR29c, hsa-mir-1302-4, hsa-mir-3657, hsa-mir-3657 and hsa-mir-4647. The top hits in the sense (+) orientation is the target microRNA miR29a and miR29c (miR29b was not identified in light of the bioinformatics parameters used for this analysis). Potential cross-hybridization targets are in the complementary (−) orientation. The top complementary microRNA sequences are poorly characterized microRNA hsa-mir-3657 and hsa-mir-4647. Both these microRNAs may represent in silico artefacts with no function. Moreover, both these sequences share a limited region of full interrupted identity (10 bp) and are unlikely to have enough affinity to be strongly modulated by steric inhibition by miR29a mimics at physiological concentrations in vivo.

### A2) in Silico Evaluation of Potential Hybridization-Dependent Liabilities for the CWT-001 Guide Strand in NCBI's Reference Sequence (RefSeq) RNA Sequences

Q-PCR analysis was performed on expression levels of potential off-target genes in primary human tenocytes after treatment with CWT-001. Potential cross-hybridization targets were identified as LAMA2, PDE7B, LRRC73, NETO1, FHL5, LINC00309, DOCK8, FILIPi, CUL9, CNOT8, LOC100132077, SCP2 and PROX2. Briefly, in the complementary orientation, suitable for cross-hybridization, multiple unique genes were identified with uninterrupted complementarity of 13-14 bp to the sequence. There were no RNA with uninterrupted complementarity over 14 bp, limiting the potential for cross-hybridization. Mutations in three genes (LAMA2, DOCK8 and SCP2) are associated with known human diseases.

B1) in Silico Evaluation of Potential Hybridization-Dependent Liabilities for CWT-001 Passenger Strand in Precursor microRNA Sequences

qPCR analysis was performed on the expression levels of miRNAs in primary human tenocytes after treatment with CWT-001. Potential cross-hybridization targets were identified as hsa-miR29a, hsa-mir-5186, hsa-mir-548at, hsa-mir-1283-2, hsa-mir-454, hsa-mir-4782, hsa-mir-515-1, hsa-mir-515-2, hsa-mir-5691. The top complementary microRNA sequences are hsa-mir-548at, hsa-mir-1283-2 and hsa-mir-515 family. All these sequences share a limited region of full interrupted identity (11 bp or less) and are unlikely to have enough affinity to be strongly modulated by steric inhibition by CWT-001 at physiological concentrations in vivo.

### B2) in Silico Evaluation of Potential Hybridization-Dependent Liabilities for CWT-001 Passenger Strand in NCBI's Reference Sequence (RefSeq) RNA Sequences

qPCR analysis was performed on the expression levels of potential off-target genes in primary human tenocytes after treatment with CWT-001. Potential cross-hybridization targets were identified as PDCD10, ITGB6, LPGAT1, BPTF, CTTNBP2, EIF4A3, KIAA1210, SLC7A11-AS1, THSD4, TNFAIP3, ANKLE2, ARL4A, ATG14, BCL11A, CCDC83, CDH4, CLVS1, DKFZp434L192, ETV7, FBXO45, KCNJ1, LINC01973, MCF2L2, METTL5, NBN, POC1B, RAVER2, RPA2, ZNF441, ZNF667 and OPCML. Mutations in nine genes (ANKLE2, BCL11A, BPTF, EIFA3, ITGB6, KCNJ1, NBN, PDCD10, POC1B, & TNFAIP3) are associated with known human diseases.

### C) Study in Human Tenocytes

To identify if any of the potential predicted interactions occurred, measurement of the expression levels of potential off-target mRNAs by qPCR in human tenocytes treated with 100 μg/ml CWT-001 for 24 hours was conducted. Of the 52 potential off-target genes only 12 were expressed in tenocytes and of these only four of the genes known to be associated with human disease (ANKLE2, SCP2, BPTF and EIFA3) were expressed in tenocytes. Significantly, treatment with CWT-001 did not alter the expression of any of the genes highlighted as being potential off-targets of CWT-001.

The data shows that treatment with CWT-001 does not produce off-target effects in primary human tenocytes.

### Example 3b—Immunogenicity Assessment of CWT-001 in Primary Human Macrophages

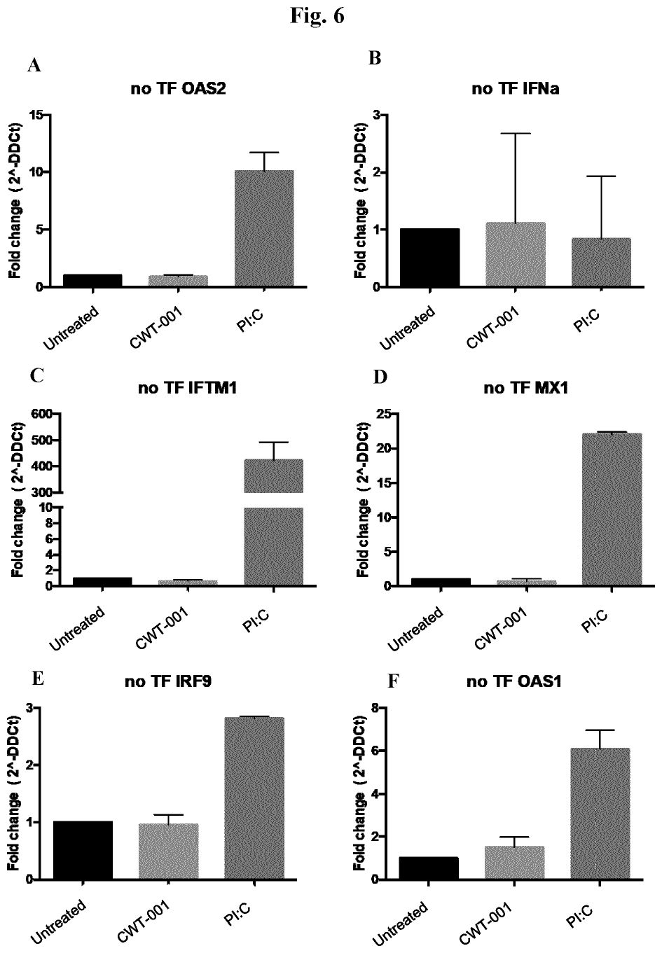

Double-stranded RNA (ds-RNA) may elicit an immune response (ref. 14). To test whether CWT-001 might elicit an immune response, cultured primary human macrophages, which as a key component of the innate immune system are sensitive to ds-RNA molecules, were treated with 0.35 μg/ml CWT-001 for 24 hrs (n=4) and the levels of the pro-inflammatory cytokine tumour necrosis factor-alpha (TNFα) measured by ELISA. The results showed no induction of TNFα in CWT-001 treated cells (FIG. 5). qPCR analysis of a panel of interferon-inducible genes (OAS2, INFα, IFTM1, MX1, IRF9 and OAS1) was additionally performed. Samples were normalised to 18S rRNA levels and expressed as fold change compared to untreated control (n=3). The qPCR analysis showed no change in expression in CWT-001-treated macrophages (FIG. 6).

Taken together, these data demonstrate that CWT-001 does not induce immunogenicity in human macrophages.

### Example 3c—Immunogenicity Assessment of CWT-001 in Mice

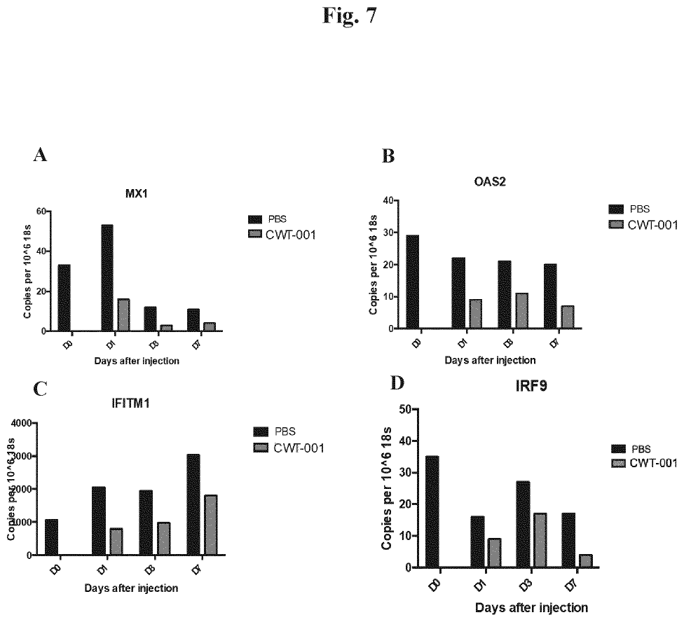

Immunogenicity of CWT-001 was tested in mice following CWT-001 injections into the patellar tendon. Animals were injected intra-tendon with 50 μl of 0.35 μg/ml of CWT-001 or PBS (n=3). The expression of known Interferon responsive genes MX1, OAS2, IFITM1 and IRF9 were measured by qPCR on days 1, 3 and 7 and compared to PBS controls. The resulting data showed no increase in Interferon responsive gene expression, suggesting that CWT-001 is non-immunogenic (FIG. 7). In fact, the data suggest that CWT-001 has anti-inflammatory activity, as reflected by lower expression levels of interferon-responsive genes in mice treated with CWT-001 compared to PBS control.

These data show that CWT-001 does not induce immunogenicity in a mouse tendon injury model, and appears to have anti-inflammatory activity.

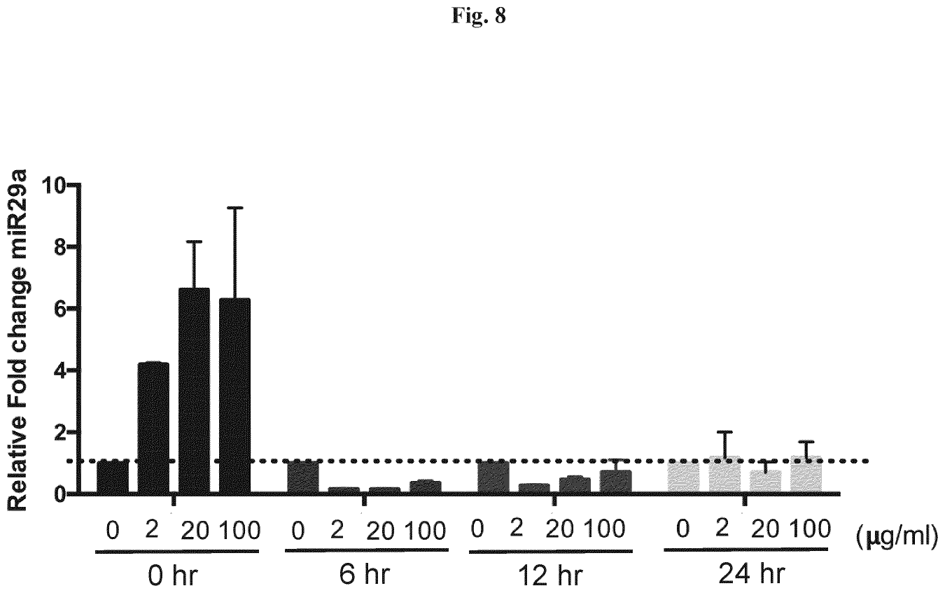

### Example 4: Pharmacokinetic/Toxicokinetics (FIGS. 8-12)

### Example 4a—Detection of CWT-001 in Preclinical Models

A two-step reverse transcription quantitative PCR (RT-QPCR) method for the detection of CWT-001 in rat and dog RNA was validated. The assay was linear between 108-103 copies in the absence of matrix RNA. Intra- and inter-assay accuracy and precision assessments testing on 7 occasions per matrix by 3 analysts demonstrated the robustness of the method in the range established.

RNA QC Pool samples for each matrix were prepared as endogenous controls to assess endogenous baseline levels. During sample analysis, RNA QC Pool samples are used for data normalisation of the QC samples and to determine the expected Ct values and determined copies numbers. RNA QC Pool samples are set to be within the following values:
Ct 24.771-26.697 and/or 6.67E+03-3.31E+04 copies/reaction rat plasma
Ct 25.195-27.496 and/or 1.38E+03-2.79E+04 copies/reaction dog plasma
Ct 23.284-24.352 and/or 5.45E+04-8.81E+04 copies/reaction for rat tendon
Ct 23.135-24.339 and/or 5.49E+04-9.88E+04 copies/reaction for dog tendon

Stability of CWT-001 in spiked tendon samples for rat and dog stored in a freezer set to maintain −80° C. was assessed and showed stability for up to 1 month.

The methods used in these studies were considered fit for extracting and measuring CWT-001 in rat and dog RNA.

### Example 4b—CWT-001 Detectability and Stability in Human Serum

In the event that CWT-001 entered the systemic circulation, the duration of CWT-001's detectability against background levels of endogenous miR29a in human serum was determined. CWT-001 was diluted to concentrations equivalent to those predicted immediately following intravenous (IV) delivery in an adult human (1:5,000 in 1 ml of serum, based on average adult blood volume of 5 litres). Samples of 0, 2, 20 and 100 μg/ml CWT-001 were incubated with fresh human serum at 37° C. for 0, 6, 12 and 24 hrs. MiR29a levels were measured by qPCR normalised to Cel39 spike in control and shown as relative expression compared to untreated samples (n=3) (FIG. 8). An increase in miR29a attributable to CWT-001 was observed only in samples that were purified less than 1 minute after addition of CWT-001, with miR29a levels returning to near untreated levels after 6 hrs. This data shows that CWT-001 is rapidly degraded to undetectable levels in human serum in vitro.

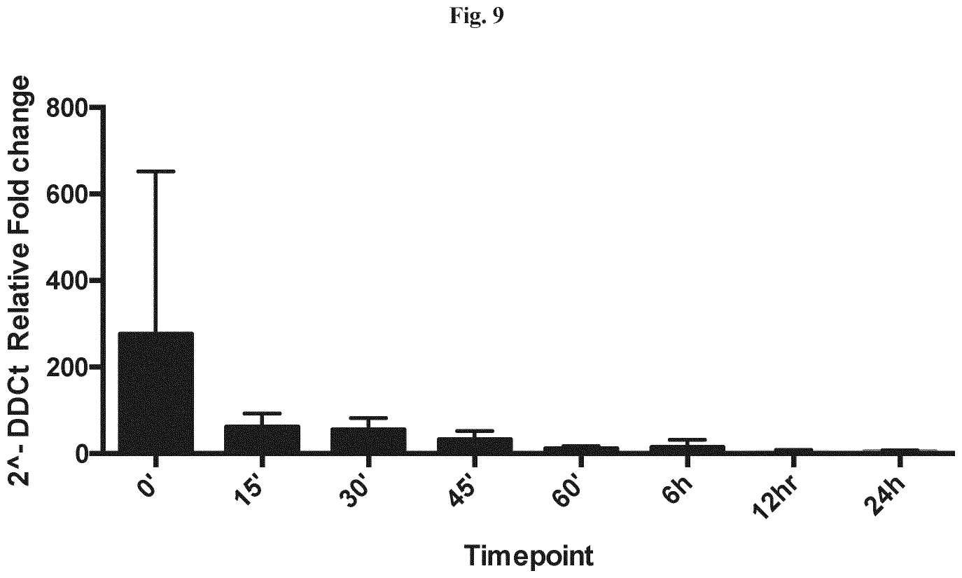

To further define the kinetics of CWT-001 in human serum samples, additional studies were performed focusing on earlier time points (0, 15, 30 and 45 minutes; 1, 6, 12 and 24 hrs). MiR29a levels were measured by qPCR, values normalised to Cel39 spike in control and shown as relative fold change compared to untreated samples (n=3) (FIG. 9). The data showed that CWT-001 levels, when measured immediately after addition to the serum, increased to a mean level of 275 times that of background, before dropping to 61 times background after 15 minutes. By 1 hour, levels had fallen to 11 times background before falling to background levels at 12 hours.

### Example 4c—Pharmacokinetics and Distribution of CWT-001 after IV Administration

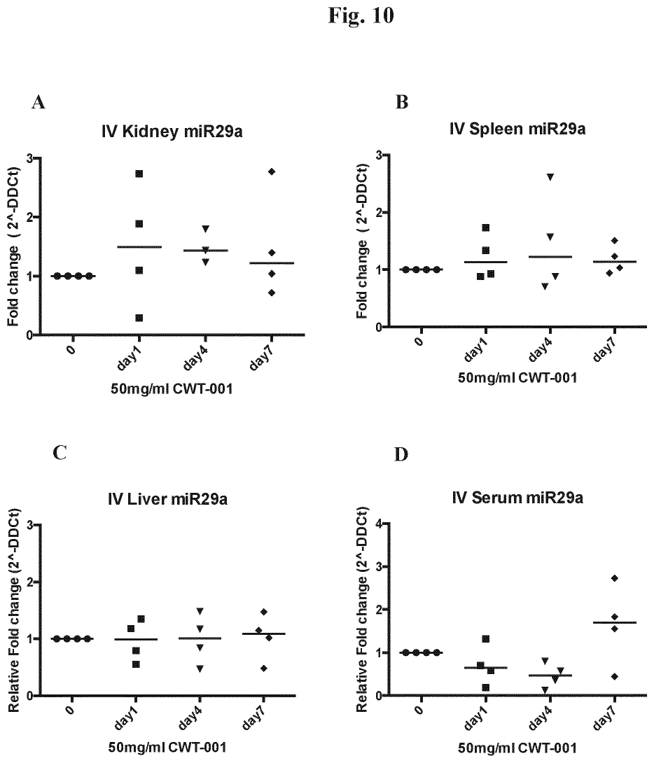

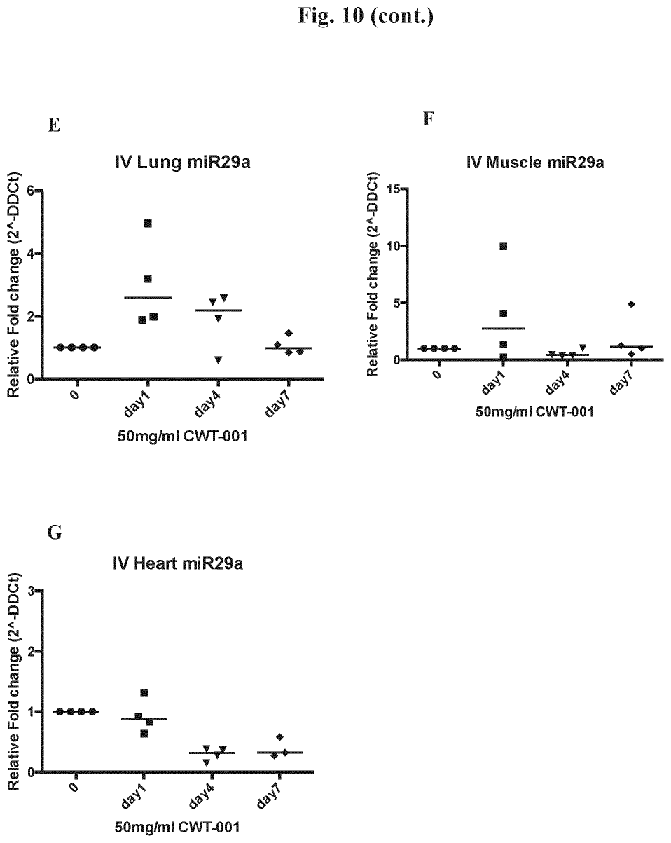

To explore the in vivo distribution of CWT-001 following intravenous injection, mice were injected with 50 μl of 50 mg/ml CWT-001, and serum, heart, liver, kidney, spleen and muscle were collected after 0, 1, 4 and 7 days. CWT-001 levels were measured using a miR29a qPCR assay, values were normalised to GAPDH and shown as relative fold-change compared to untreated controls. Serum samples were normalised to Cel39 spiked in control and shown as fold-change relative to untreated control (n=4) (FIG. 10). This data showed little or no increase in miR29a levels in serum or organs compared to the saline control. A small increase in miR29a levels in the lungs was observed on day 1, which had returned to baseline by day 4.

These data show that CWT-001 has negligible distribution in tissues following intravenous administration, even when administered at 25 times of the level of a typical clinical dose (i.e., 25 times of 50 μl of 2 mg/ml, which is the mouse equivalent of a human clinical dose of 1 ml of 2 mg/ml CWT-001).

### Example 4d—Systemic Exposure of CWT-001 in Circulation after Intra-Tendon Injection

Measurement of CWT-001's systemic exposure was conducted in mice injected with 25 μl of 2 mg/ml CWT-001 or PBS control into the patella tendon of mice (n=4 per group) and blood was collected at 0, 15 and 30 minutes and 1, 2, 4, 6, 24 and 72 hrs. CWT-001 levels were measured using a miR29a qPCR assay, values normalised to Cel39 spike in control and expressed as fold-change relative to PBS control (n=4).

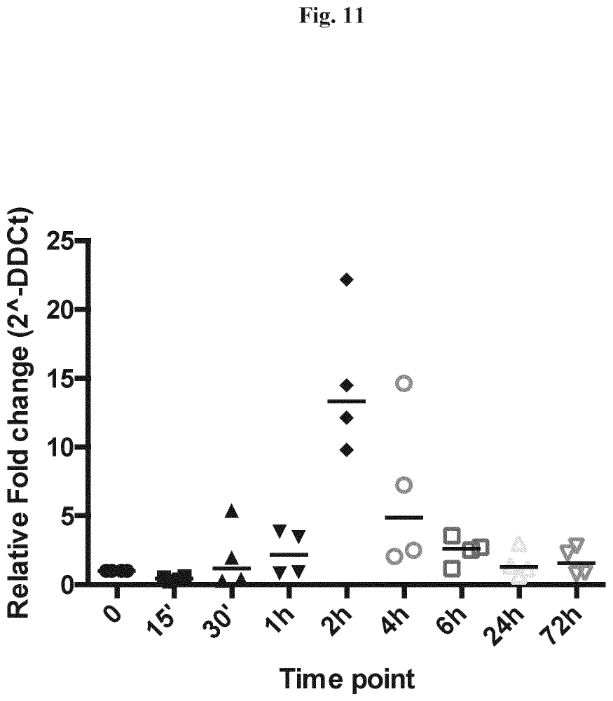

Following intra-tendon administration, an increase in miR29a levels in blood at 2 and 4 hours post-injection was seen. miR29a levels returned to background levels at 6 hours (FIG. 11). This data suggests that while CWT-001 does enter the circulation after intra-tendon injection, it is rapidly cleared, presumably by degradation by serum nucleases and excretion through the kidneys. The absolute levels of endogenous miR29a observed systemically are very low (e.g., as compared to the concentrations present in cells). Therefore, the relative increase in systemic miR29 levels of 5-14 fold observed at 2, and 4 hours post CWT-001 injection (FIG. 11) translates into still very low absolute systemic miR29a levels. Furthermore, as noted above, most of the serum CWT-001 is rapidly cleared. Less than 1% of the CWT-001 is ultimately taken up by cells via the serum. Overall, this means that only very low absolute levels of administered CWT-001 will enter non-target cells via systemic exposure. The observed systemic increase in CWT-001 is therefore unlikely to exert any pharmacodynamic action in cells apart from the target cells within the tissue into which CWT-001 is administered (e.g., the tendon).

This data demonstrates that CWT-001 has limited systemic distribution following intra-tendon injection.

### Example 4e—Distribution of CWT-001 after Intra-Tendon Administration

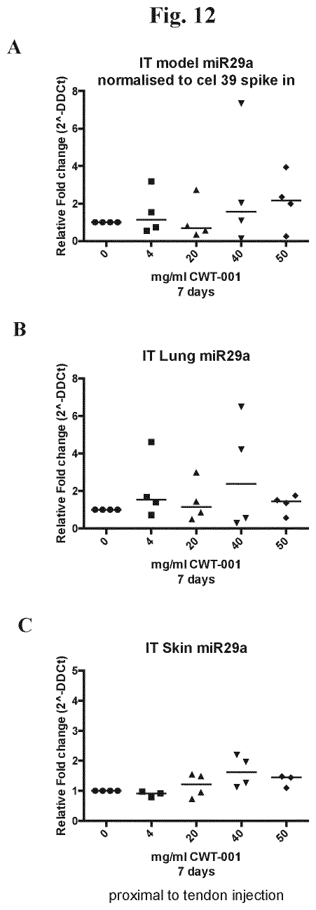

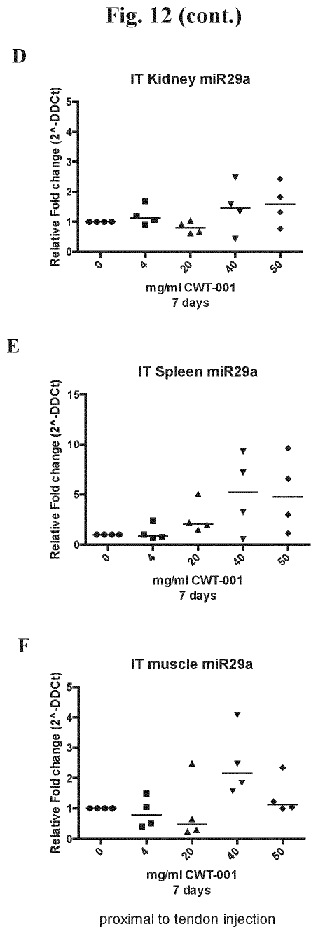

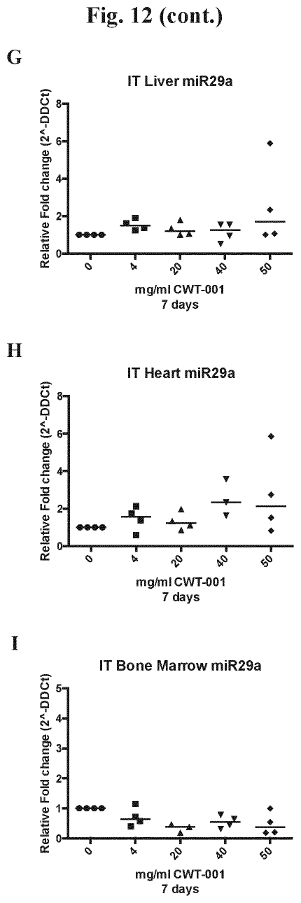

To understand CWT-001 tissue distribution after intra-tendon injection, patellar tendons of mice were injected with 50 μl of 4, 20, 40 and 50 mg/ml of CWT-001 or PBS control. Organs and peripheral blood were collected after 7 days. CWT-001 levels were measured using a miR29a qPCR assay, values normalised to GAPDH and shown as relative fold-change compared to untreated controls. Serum samples were normalised to Cel39 spiked in control and shown as fold-change relative to placebo (n=4). The resulting data (FIG. 12) showed no significant increases in miR29a levels in any organs at any concentrations compared to placebo. A trend towards significance was noted at the two highest doses in the spleen, which are higher than any typical equivalent human clinical doses.

These data show that CWT-001 has negligible distribution in tissues even at the highest doses, which are up to 25 times the highest typical equivalent human clinical dose.

### Example 4f—Toxicokinetics in Rats Following Intra-Tendon Administration in a Single Dose Toxicity Study

Toxicokinetic assessment was incorporated into the design of the single dose toxicity study of CWT-001 by intra-tendinous injection in rats with a 14 day recovery period (Example 5c).

Rats were treated with a 30 μL injection of saline control, 10 mg/ml or 50 mg/ml (i.e., 0, 0.3 or 1.5 mg total dose) of CWT-001. Blood samples were collected from the jugular vein from 3 animals per sex per group at 0.5, 1, 2, 4, 8 and 24 hours post dose and plasma prepared. Plasma samples were analysed for the concentration of CWT-001 using a validated analytical procedure (RT-QPCR).

Toxicokinetic parameters were estimated using a non-compartmental approach consistent with the intra-tendon route of administration in this study. A summary of the toxicokinetic parameters is provided in Table 2 below.

TABLE 2
Toxicokinetic Parameters of CWT-001 in Male and Female Rat Plasma

| | | | | Cₘₐₓ/D | | AUC (0-t)/D |
|---|---|---|---|---|---|---|
| | Dose | Tₘₐₓ | Cₘₐₓ (SE) | (copies/mL)/ | AUC (0-t) (SE) | (copies · h/mL)/ |
| Gender | (mg) | (h) | (copies/mL) | (mg) | (copies · h/mL) | (mg) |
| Male | 0.3 | 0.5 | 9.93E+09 | 3.31E+10 | 1.56E+10 | 5.20E+10 |
| | | | (2.88E+09) | | (5.58E+09) | |
| | 1.5 | 0.5 | 3.43E+10 | 2.29E+10 | 5.37E+10 | 3.58E+10 |
| | | | (9.73E+09) | | (8.29E+09) | |
| Female | 0.3 | 0.5 | 1.01E+09 | 3.36E+09 | 1.80E+09 | 6.00E+09 |
| | | | (1.01E+09) | | (7.73E+08) | |
| | 1.5 | 0.5 | 1.32E+10 | 8.80E+09 | 1.70E+10 | 1.13E+10 |
| | | | (3.70E+09) | | (1.10E+09) | |

n = 9 for males and female, 3 animals per sex per timepoint were sampled; one composite TK profile/sex/dose.

Plasma CWT-001 concentration vs time profiles displayed a rapid absorption phase (Tₘ. was 0.5 hours in all profiles). Plasma CWT-001 concentrations decreased in a multi-phasic manner up to 4 hours post-dose, after which, in females, concentrations slightly increased up to 8 hours (last quantifiable concentration, 0.3 mg) or remained at a relatively steady state until the end of the sampling period (24 hours) (1.5 mg). In males, concentrations at both doses remained at a steady state after 4 hours until the end of the sampling period (24 hours). With an increase in dose from 0.3 mg to 1.5 mg, systemic exposure to CWT-001 increased in a generally less than dose-proportional manner in females, and greater than dose-proportional manner in males. Systemic exposure to CWT-001 was greater in males than in females at both 0.3 mg and 1.5 mg dose levels.

### Example 4g—Toxicokinetics in Dogs Following Intra-Tendon Administration in a Single Dose Toxicity Study

Toxicokinetic assessment was incorporated into the design of the single dose toxicity study of CWT-001 by intra-tendinous injection in dogs with a 14 day recovery period (Example 5d).

Dogs were treated with a 250 μL injection of saline control, 10 mg/ml or 50 mg/ml of CWT-001 (i.e., 0, 2.5 or 12.5 mg total dose). Blood samples were collected from the jugular vein from 3 animals per sex per group at 0.5, 1, 2, 4, 6 and 48 hours post dose and plasma prepared. Plasma samples were analysed for the concentration of CWT-001 using a validated analytical procedure (RT-QPCR).

Toxicokinetic parameters were estimated using a non-compartmental approach consistent with the intra-tendon route of administration in this study. A summary of the toxicokinetic parameters is provided in the following Table.

TABLE 3
Individual and Mean Toxicokinetic Parameters
of CWT-001 in Male and Female Dog Plasma

| | Dose | | | | Cₘₐₓ/D | | AUC (0-t)/D |
|---|---|---|---|---|---|---|---|
| | Level | | Tₘₐₓ | Cₘₐₓ | (copies/mL)/ | AUC (0-t) | (copies · h/mL)/ |
| Gender | (mg) | Animal | (h) | (copies/mL) | (mg) | (copies · h/mL) | (mg) |
| Male | 2.5 | n | 3 | 3 | 3 | 3 | 3 |
| | | Mean* | 1 | 2.49E+09 | 9.97E+08 | 4.63E+09 | 1.85E+09 |
| | | SD | — | 1.09E+09 | 4.37E+08 | 2.89E+09 | 1.16E+09 |
| | 12.5 | n | 5 | 5 | 5 | 4 | 4 |
| | | Mean* | 0.5 | 5.23E+09 | 4.19E+08 | 7.87E+09 | 6.30E+08 |
| | | SD | — | 2.16E+09 | 1.73E+08 | 5.33E+09 | 4.26E+08 |
| Female | 2.5 | n | 3 | 3 | 3 | 1 | 1 |
| | | Mean* | 0.5 | 1.26E+09 | 5.06E+08 | 1.44E+09 | 5.78E+08 |
| | | SD | — | 2.64E+08 | 1.05E+08 | — | — |
| | 12.5 | n | 5 | 5 | 5 | 5 | 5 |
| | | Mean* | 0.5 | 7.33E+09 | 5.86E+08 | 8.94E+09 | 7.16E+08 |
| | | SD | — | 6.57E+09 | 5.26E+08 | 3.55E+09 | 2.84E+08 |

“—” Value not applicable.
*Median reported for Tₘₐₓ.
^{a} AUC up to 6 hours; value calculated is used for the calculation of descriptive statistics to aid interpretation.
ND = Not determined, less than three consecutive concentrations were quantified.

Plasma CWT-001 concentration versus time profiles displayed a rapid absorption phase (median Tₘₐₓ of 0.5 or 1 hours). This was followed by a bi-phasic decline in concentrations firstly in a rapid manner up to 1 or 2 hours post dose, and secondly at a slower rate up to the last quantifiable concentration (except for females dosed at 12.5 mg, which showed a slight increase in concentrations between 4 and 6 hours post dose). With an increase in dose from 2.5 mg to 12.5 mg, systemic exposure to CWT-001 increased in a generally dose-proportional manner in females, and less than dose-proportionally in males. Systemic exposure to CWT-001 was greater in males than females at a dose level of 2.5 mg, but comparable between sexes at 12.5 mg.

### Example 5: Toxicology

### Example 5a—Tolerability Study of CWT-001 by a Single Intra-Tendinous Injection in Rats

The objectives of this study were to determine the potential toxicity of CWT-001 when given to rats by intra-tendinous injection, with the aim of establishing and confirming a maximum feasible dose (MFD). The study design is set out in Table 4 below.

TABLE 4
Experimental Design of Rat Single Intra-
Tendinous Injection Tolerability Study

| | | | | Dose | | |
|---|---|---|---|---|---|---|
| | | Dose | Dose | Concen- | | |
| Group | | Level | Volume | tration | Animal Numbers | |
| No. | Treatment | (mg) | (μL) | (mg/mL) | Males | Females |
| 1 | CWT-001 | 0.3 | 30 | 10 | — | 1501-1503 |
| 2 | CWT-001 | 0.75 | 30 | 25 | — | 2501-2503 |
| 3 | CWT-001 | 1.5 | 30 | 50 | — | 3501-3503 |
| 4 | CWT-001 | 1.5 | 30 | 50 | 4001-4003 | — |

— = Not applicable
PBS (138 mM sodium chloride; 8 mM sodium phosphate) pH 6.8-7.6 was used to dissolve the test item

Each animal received a single intra-tendinous administration (30 μL) of CWT-001 into the right Achilles tendon using a 0.5 mL insulin syringe. Animals were anaesthetised using isoflurane, and the tendon was exposed using a small surgical incision to the hindlimb to allow placement of CWT-001. Animals were sutured closed after the dose was administered.

The highest dose level (1.5 mg) represented the maximum feasible dose that could be administered by this route, limited by the solubility of the test item and the volume that could reasonably be injected into the tendon. The following parameters and end points were evaluated in this study: clinical observations, body weights and gross necropsy findings. There were no clinical signs, changes in body weight or gross necropsy findings that were considered to be related to CWT-001.

In conclusion, administration of CWT-001 by single intra-tendinous administration was well tolerated in Han Wistar rats up to 1.5 mg with no clinical observations, changes in body weight or gross necropsy findings observed.

### Example 5b—Tolerability Study of CWT-001 by a Single Intra-Tendinous Injection in Dogs

The objectives of this study were to determine the potential toxicity of CWT-001 when given as a single intra-tendinous injection to dogs and to provide data to support dose level selection for a follow-on 14 day toxicity study. The study design is set out in Table 5 below.

TABLE 5
Experimental Design of Dog Single Intra-
Tendinous Injection Tolerability Study

| | | | | Dose | | |
|---|---|---|---|---|---|---|
| | | Dose | Dose | Concen- | | |
| Group | | Level | Volume | tration | Animal Numbers | |
| No. | Test Item | (mg) | (μL) | (mg/mL) | Male | Female |
| 1 | CWT-001 | 12.5 | 250 | 50 | 1001 | 1501 |

Vehicle = PBS (138 mM sodium chloride; 8 mM sodium phosphate)

Each animal received a single intra-tendinous administration (250 μL) of CWT-001 into the right Achilles tendon using a syringe. Animals were anaesthetised using isoflurane, and the tendon was exposed using a small surgical incision to the hindlimb to allow placement of CWT-001. Animals were sutured closed after the dose was administered.

The dose level used (12.5 mg) represented the maximum feasible dose that could be administered by this route, limited by the solubility of the test item and the volume that could reasonably be injected into the tendon. The following parameters and end points were evaluated in this study: clinical observations, body weights, food consumption, clinical pathology parameters (haematology, coagulation and clinical chemistry), gross necropsy findings and organ weights.

There were no CWT-001 clinical observations and no changes in body weight, food consumption, haematology, coagulation or clinical chemistry parameters. Additionally, there were no gross necropsy findings or organ weight changes that were considered to be related to CWT-001. In conclusion, administration of CWT-001 by single intra-tendinous administration in beagle dogs, was well tolerated at 12.5 mg with no clinical observations noted, no changes in body weights, food consumption or clinical pathology parameters, and no gross pathology or organ weight findings.

### Example 5c—a Single Dose Toxicity Study of CWT-001 by Intra-Tendinous Injection in Rats with a 14 Day Recovery Period

The objectives of this study were to determine the potential toxicity of CWT-001 when given via a single intra-tendon injection to rats and to evaluate the potential reversibility of any findings over a 14 day recovery period following the injection. In addition, the toxicokinetic characteristics of CWT-001 were determined. The study design is set out in Table 6 below.

TABLE 6
Experimental Design of Single Dose Toxicity Study by Intra-
Tendinous Injection in the Rat with 14 day Recovery Period

| | | Dose | Dose | Dose | Number of Animals | | | |
|---|---|---|---|---|---|---|---|---|
| Group | | Level | Volume | Concentration | Main Study | | Recovery/Satellite | |
| No. | Treatment | (mg) | (μL) | (mg/mL) | Males | Females | Males | Females |
| 1 | Control | 0 | 30 | 0 | 10 | 10 | 5 | 5 |
| 2 | CWT-001 | 0.3 | 30 | 10 | 10 | 10 | 4 | — |
| 4 | CWT-001 | 1.5 | 30 | 50 | 10 | 10 | 5 | 5 |

Control = PBS (100 mM sodium chloride; 20 mM sodium phosphate) pH 6.8-7.6
— = Not applicable.

The following parameters and end points were evaluated in this study: clinical observations, body weights, food consumption, ophthalmology, behavioural observations (Irwin scores), respiratory measurements, clinical pathology parameters (haematology, coagulation, clinical chemistry, and urinalysis), bioanalysis and toxicokinetic parameters, gross necropsy findings, organ weights and histopathological examinations.

The highest dose level (1.5 mg) represented the maximum feasible dose that could be administered by this route, limited by the solubility of the test item and the volume that could reasonably be injected into the tendon of a rat (see Example 5a). All formulation analysis results were found to be within or equal to the acceptance criteria of ±10% (individual values within or equal to ±15%) of their theoretical concentrations.

There were no unscheduled deaths throughout the course of this study. There were no clinical signs, changes in body weights or food consumption, ophthalmoscopy findings, behavioural observations, changes in respiratory parameters, organ weight changes or gross or histopathological findings that were considered to be related to CWT-001. At 0.3 or 1.5 mg, there was a slight but dose dependent decrease in red cell mass (red blood cells, haematocrit and haemoglobin) and an increase in reticulocytes on Day 3 in both males and females. All changes showed recovery by Day 15.

In conclusion, administration of CWT-001 by single intratendinous administration was well tolerated in Han Wistar rats up to 1.5 mg and was associated with transient slightly lower red blood cell mass and increases in reticulocytes on Day 3, which showed recovery by Day 15. There were no other in life or any histopathology findings related to CWT-001.

Based on the results of this study which was dosed at the maximum feasible dose, 1.5 mg CWT-001 was considered to be the No Observed Adverse Effect Level (NOAEL) in rats.

### Example 5d—a Single Dose Toxicity Study of CWT-001 by Intra-Tendinous Injection in Dogs with a 14 Day Recovery Period

The objectives of this study were to determine the potential toxicity of CWT-001 when given as a single intra-tendinous injection to dogs, and to evaluate the potential reversibility of any findings. In addition, the toxicokinetic characteristics of CWT-001 were determined.

Each animal received a single dose of CWT-001, with main study animals being euthanised on Day 3 and recovery animals on Day 15. The study design is set out in Table 7 below.

TABLE 7
Experimental Design of Single Dose Toxicity Study by Intra-
Tendinous Injection in the Dog with 14 day Recovery Period

| | | | | | | Number of Animals | | | |
|---|---|---|---|---|---|---|---|---|---|
| | | Dose | Dose | Dose | Adjusted Dose | Main | | | |
| Group | | Level | Volume | Concentration | Concentration | Study | | Recovery | |
| No. | Treatment | (mg) | (μL) | (mg/mL) | (mg/mL) | M | F | M | F |
| 1 | Control | 0 | 250 | 0 | 0 | 3 | 3 | 5 | 5 |
| 2 | CWT-001 | 2.5 | 250 | 10 | 14.2 | 3 | 3 | — | — |
| 3 | CWT-001 | 12.5 | 250 | 50 | 71 | 3 | 3 | 5 | 5 |

Control Item = PBS (138 mM sodium chloride; 8 mM sodium phosphate) pH 6.8-7.6
M = Males,
F = Females,
— = Not applicable

The following parameters and end points were evaluated in this study: clinical observations, body weights, food consumption, ophthalmology, cardiovascular data, clinical pathology parameters (haematology, coagulation, clinical chemistry, and urinalysis), bioanalysis (plasma and tendon) and toxicokinetic parameters, gross necropsy findings, organ weights, and histopathological examinations. The highest dose level (12.5 mg) represented the maximum feasible dose that could be administered by this route, limited by the solubility of the test item and the volume that could reasonably be injected into the tendon of a dog (see Example 5b). All formulation analysis results were found to be within or equal to the acceptance criteria of ±10% (individual values within or equal to ±15%) of their theoretical concentrations.

There were no unscheduled deaths throughout the course of this study. There were no clinical signs, changes in body weights or food consumption, ophthalmoscopy findings, electrocardiology findings, clinical pathology findings (haematology, coagulation, clinical chemistry and urinalysis), organ weight changes or gross or histopathological findings that were considered to be related to CWT-001.

In conclusion, administration of CWT-001 by single intra-tendinous administration was well tolerated in beagle dogs up to 12.5 mg and was only associated with findings related to the administration procedure. There were no in life or histopathology findings related to CWT-001. Based on the results of this study which was dosed at the maximum feasible dose, 12.5 mg CWT-001 was considered to be the No Observed Adverse Effect Level (NOAEL) in dogs.

### Example 6: Safety Pharmacology

Safety Pharmacology endpoints were incorporated into the design of the rat and dog single dose toxicity studies (Examples 5c and 5d, respectively) to assess central nervous system, respiratory and cardiovascular effects

### Example 6a—Central Nervous System (Modified Irwin Observations) in Rats

The first 6 male main study animals from each group were observed at 2, 24 and 48 hours post dose. The following parameters were included in the observations:
Occurrence of vocalisation, stereotypies, aggressiveness, abnormal gait, straub tail, tremor, twitches, convulsions, body posture, sedation, catalepsy, ptosis, exophthalmos, salivation, lacrimation, piloerection, abnormal respiration, defecation, urination and death.
Increase or decrease of spontaneous activity, touch response, body tonus and pupil size. Increase of sniffing, grooming, scratching and rearing.
Decreased pinna reflex, abnormal traction response and abnormal grip strength. Any additional symptoms observed were also noted.
Frequencies of animals exhibiting symptoms and the severity of symptoms were recorded.
Pupil size: measured using a guidance chart to estimate the size in millimetres (pre-dose pupil size data was recorded on the day of dosing).
Body temperature: measured by using a probe inserted approximately 2 cm past the anal sphincter. Pre-dose body temperature was also recorded. To reduce measurement variability a temperature habituation trial was conducted by inserting the temperature probe prior to the recording of the predose measurements, however, no body temperature data was collected during this trial.

There were no findings noted during the Modified Irwin Observations that were considered to be related to administration of CWT-001. There were occasional instances of decreased grip strength observed in some animals at some time points but as these findings were also observed in the control group and at a single time point, they are deemed unrelated to administration of CWT-001. There were no changes in pupil size or body temperature that were related to administration of CWT-001.

### Example 6b—Respiratory Measurements in Rats

On the test day, animals were removed from their home cage and placed in a whole body plethysmograph chamber for approximately 45 minutes, with the last 15 minutes of this period classified as the control period. The animals were then removed from the chambers for dosing of control or test item. Measurements were then taken from 2-4, 24 and 51 hours post dose, animals were placed in the chamber to acclimate for at least 30 minutes before any measurements. The effects of chamber temperature and humidity on tidal volume were compensated for within the plethysmograph chamber. Ventilatory parameters were determined from the respiratory signal. Sampling frequency was fixed at 500 Hz.

There were no changes in respiratory parameters that were considered to be related to administration of CWT-001. Incidental statistically significant variations in respiratory parameters were present sporadically during the study, however these were deemed to be due to variability in the control group at the specific time point, or were minimal and/or transient changes, therefore considered unrelated to administration of CWT-001.

### Example 6c—Cardiovascular Assessment in Dogs

Cardiovascular assessments were measured pre-treatment (prior to Day 1) for a period at least 26 hours, on Day 2 of the dosing period for at least 20 hours (acquiring data between 24 hours to 44 hours post dose) and on Day 14 of the recovery period. The ECG and blood pressure of each animal was recorded by Jacketed External Telemetry (JE™) System in conjunction with PA-C10-TOX-LA transmitter (Data Sciences International (DSI™), St Pauls, MN, USA). Prior to the first recording session, all animals were acclimatised to the JET jackets and observed individually to ensure that they tolerated the procedure. On the days of data acquisition, the animals were removed from their home pens and taken to a procedure room for placement of the ECG leads and fitting of the jacket (and collar if required). The areas required for electrode placement were shaved and prepared appropriately. Arterial blood pressure, heart rate and lead II ECG variables were acquired continuously using a 1 minute logging rate. For pre-treatment and recovery, ECG and BP recordings were carried out for at least 26 hours. For Day 2, ECG and BP recordings were carried out for at least 20 hours (acquiring data between 24 to 44 hours post dose), (T=0 equates to end of administration of the test item). Animals were not subject to restraint except where dosing procedures were performed. Prior to the initiation of the dosing phases of the study, pretreatment telemetry data acquisitions of at least 26 hours duration were recorded from all animals. Sections of these pretreatment signals were reviewed, as deemed necessary, for any ECG morphology abnormalities, quality of signal and normality of heart rate, arterial blood pressure and ECG values (including QTca calculations [QT corrected for heart rate]).

The following parameters were reported:
Systolic (SBP), diastolic (DBP) and mean arterial blood pressure (MAP), and heart rate (HR) derived from the blood pressure waveform.
ECG parameters (RR, PR, QRS, QT corrected for individual animals, QTca intervals QA interval [derived from the blood pressure and ECG waveforms]) were analysed. Any lead II ECG abnormalities or arrhythmias observed were printed and reviewed.

There were no changes in cardiovascular parameters or waveform morphology throughout the course of the study that were considered to be related to administration of CWT-001. There were occasional or transient observations in individual animals that were not considered to be test item related since they were also observed in control animals or considered to be due to normal inter-animal variability.

### Example 6d—Reproductive and Developmental Toxicity

The single dose toxicity studies in the rat and dog did not reveal any treatment related organ weight or histopathological changes of reproductive organs including the testes and ovaries.

### Example 6e—Local Tolerance

Local tolerance has been assessed in the single dose toxicity studies in both rat and dog and did not reveal any unexpected effects at the site of injection.

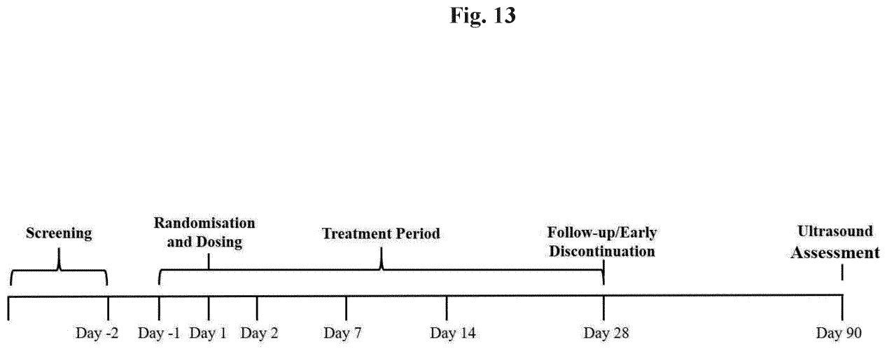

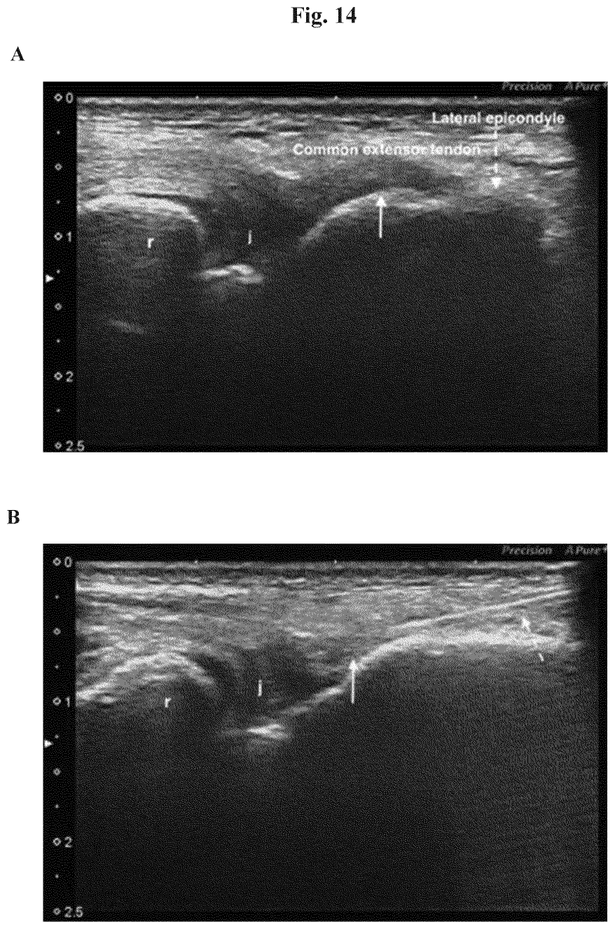

### Example 7: Administration to Human Subjects with Lateral Epicondylitis (FIGS. 13 and 14)

The results from the examples described above indicate that the miR-29 compounds provided herein are safe and effective in reducing or inhibiting collagen 3 expression in vivo, including in mouse models of tendinopathy. Additionally, the inventors have previously shown that an equine collagenase-induced tendinopathy (SDFT) model reflects human tendinopathy, with reduced expression of miR-29a after tendon injury and a concomitant increase in col3:col1 ratio. A single intralesional injection of a miR-29 compound (100 nM, which translates to around 2 μg/ml in a total volume of 1.5 ml, i.e., a total dose of 3 μg) reduced collagen 3 expression and lesion size, and improved tendon structure (see ref. 3). Together, these data provide a rationale for safe and effective human therapy using miR-29 compounds, including the miR-29 compounds provided herein.

A phase 1, randomised, double-blind, placebo-controlled study evaluating the safety, tolerability and pharmacokinetics of single ascending doses of CWT-001 injections in subjects with lateral epicondylitis was conducted (see FIG. 13 for study design).

The current 2nd line (following physiotherapy) standard of care treatment of lateral epicondylitis remains local injection of corticosteroid to the area of tendon pain, even though the long-term benefit remains questionable. A local injection of CWT-001 to the area of tendon damage of the common extensor origin of the elbow is therefore in keeping with current standard of care but shows improved safety and efficacy. An ultrasound guided injection to the area of tendon damage is used, as ultrasound examination is often used as the imaging modality of choice not only to assess the status of the tendons but also to guide injection therapy (ref. 15).

The primary objective of the study is to determine the safety and tolerability of single ascending doses of CWT-001 in human subjects with lateral epicondylitis.

The secondary objective of the study is to determine the single dose pharmacokinetics (PK) of CWT-001 administration in human subjects with lateral epicondylitis, and to assess the efficacy of CWT-001 administration in human subjects with lateral epicondylitis.

The primary endpoint of the study is the comparison of safety data between CWT-001 versus placebo as measured by incidence of adverse events (AEs), clinical laboratory abnormalities, changes in vital signs (blood pressure, temperature, respiratory rate, and pulse rate), 12-lead electrocardiogram (ECG) parameters, physical examinations and skin score assessment at 14 days post injection.

The secondary endpoints of the study are:
The plasma PK concentration of data as shown by maximum plasma concentration (Cₘₐₓ), time to Cₘₐₓ (tₘₐₓ), area under the plasma vs. concentration-time curve (AUC).
Efficacy of a single dose of CWT-001 on elbow pain as measured using Visual Analogue Scale (VAS) from pre-dose on Day 1 to Day 14, Day 28 and Day 90.
Efficacy of a single dose of CWT-001 on disability and symptoms measured using the Disabilities of the Arm, Shoulder, and Hand (DASH) Score from pre-dose on Day 1 to Day 14, Day 28 and Day 90.
Efficacy of a single dose of CWT-001 on pain and disability as measured by the Patient Rated Tennis Elbow Evaluation (PRTEE) from pre-dose on Day 1 to Day 14, Day 28 and Day 90.
Efficacy of a single dose of CWT-001 on lateral elbow tendons as measured by change from baseline ultrasound assessment from pre-dose on Day 1 to Day 28 and Day 90.

### Dose Rationale

In light of the experiments described above, miR-29 doses in the μg and low mg range are expected to be effective and well tolerated in animal species, and can be administered in volumes that can easily and safely be injected (e.g., around 1 ml). These dose levels also confirm the mechanism of action targeting collagens, e.g., in improving tendon healing in the animal models used. Single dose extended toxicity studies via intra-tendinous administration were conducted with CWT-001 in the rat (up to 1.5 mg, see Example 5a) and dog (up to 12.5 mg, see Example 5b) and the no-observed-adverse-effect level for each study was the maximum feasible dose. Preclinical assessments of single intra-tendon administration of CWT-001 did not result in adverse findings at levels that were 41 and 25 times the respective doses in rat and dog compared to a human clinical dose of 2 mg, for example.

Subjects were divided into cohorts (8 subjects per cohort) and randomised to receive a single 1 ml dose of CWT-001 (a 5 mg/mL solution of CWT-001 in PBS and EDTA was diluted to the various final concentrations using normal saline, i.e. 0.9% w/v NaCl) or matched placebo (normal saline, i.e. 0.9% w/v NaCl) (3:1) administered via intra-tendon injection. The starting dose of CWT-001 is 200 μg/mL. The dose was escalated to 500 μg/mL and then 1500 μg/mL. An additional dose of (up to) 4500 μg/mL was planned to be administered if no improvement in VAS pain assessment was seen, however this was not necessary.

In each cohort, no more than 2 subjects on the first dosing day (1 active; 1 placebo) were dosed, such that no more than 1 subject received an active CWT-001 dose for the first time at each dose level. Following assessment of the safety and tolerability of the previous dosed sentinel subjects, dosing was continued in the remaining subjects (6 subjects; 5 active, 1 placebo) at the same dose level in each cohort.

Doses were administered in an escalating manner, following satisfactory review of all safety, tolerability, plasma PK, and efficacy data (where available) from lower doses. Doses were determined based on the ongoing evaluation of the safety, tolerability, plasma PK, and efficacy data (where available). All doses were given as a 1 ml injection under ultrasound guidance into the affected area.

Ultrasonography was applied using a 5-7.5 MHz linear array transducer on a LOGIQ E9 Ultrasound System. The probe was placed along the lateral aspect of the elbow parallel to the longitudinal axis of the common extensor tendon. The radial head, radiohumeral joint, lateral epicondyle, and common extensor tendon was visualised. The skin was sterilised and the USG probe enclosed in a sterile cover. After visualisation of the tip of the needle at the exact site (hypointense area), 1 mL of CWT-001 or placebo was injected (see FIG. 14).

CWT-001 was administered to the tendon using the “peppering technique” (ref. 11). The “peppering technique” is an injection method whereby after the needle is inserted into the tender area, multiple small injections are performed by withdrawing, redirecting and reinserting the needle without emerging from the skin. In the injured/chronic tendon situation this may be preferable to stimulate a healing response.

The study population included 24 subjects. Each subject met all the inclusion criteria and none of the exclusion criteria for this study.

Subjects meeting the following criteria that were included in the study:
1. Subject is male or female, of any ethnic origin.
2. Subject is aged between 18 to 70 years, inclusive, with no evidence of skeletal immaturity.
3. Subject has a body mass index (BMI) of 18 to 35 kg/m², inclusive.
4. Subject is >50 kg.
5. Subject has a clinical diagnosis of lateral epicondylitis.
6. Aside from lateral epicondylitis and any conditions listed under the exclusion criteria below, the subject is otherwise healthy as determined by a responsible physician, based on medical history, physical examinations, concomitant medication, vital signs, 12-lead ECGs and clinical laboratory evaluations. Laboratory values may be re-tested once at the discretion of the Investigator.
7. Subject's symptoms can be reproduced with resisted supination or wrist dorsiflexion (as confirmed by tenderness at lateral epicondyle and positive pick up back of chair sign).
8. Subject's symptoms have persisted for at least 6 weeks to 6 months, despite conservative treatment that includes one or a combination of:
   a. Physical therapy
   b. Splinting
c. NSAIDs
9. Subject is independent, ambulatory, and can comply with all post-injection evaluations and visits.
10. Male subjects must use a condom during the trial and for 3 months after their final dose of trial medication, if their partner is a woman of childbearing potential. In addition, their female partner of childbearing potential must use an additional method of highly effective contraception (contraceptive methods that can achieve a failure rate of less than 1% per year when used consistently and correctly are considered as highly effective birth control methods) from 1 month prior to dosing until 3 months following dosing.
11. Female subjects of childbearing potential must agree to use a highly effective method of contraception from 1 month pre-dose in combination with male partner's use of a condom during the trial and for 3 months post-dose. Subjects must have a negative pregnancy test at screening and Day 1.
12. Provision of giving written informed consent, which includes compliance with the requirements and restrictions listed in the consent form.

Subjects with any of the following were excluded from study participation:
1. Subject has undergone previous corticosteroid injection therapy to the affected elbow in less than 6 months prior to enrolment.
2. Subjects unwilling or unable to discontinue use of pain medication (opiate or NSAID) from at least 1 week prior to IMP administration.
3. Subject has received previous PRP injection to the affected elbow.
4. Subject uses or has recent use of medications known to affect the skeleton (e.g., glucocorticoid usage >5 mg/day, fluoroquinolone antibiotics).
5. Subject has undergone surgical intervention to the affected elbow for the treatment of lateral epicondylitis.
6. Subject has a positive medical history of any of the following:
   a. Medial epicondylitis
   b. Radial tunnel syndrome
   c. Carpal tunnel syndrome
   d. Septic or gouty arthritis in the affected elbow within the previous 2 years prior to enrolment
   e. Cervical radiculopathy within the previous 6 months prior to enrolment
   f. Trauma to the affected elbow within the past 6 weeks
   g. Neuromuscular or primary/secondary muscular deficiency, which limits the ability to perform functional measurement (e.g., grip strength test)
   h. Coincidental rheumatologic, inflammatory diseases, including, but not limited to axial spondyloarthritis, psoriatic arthritis and rheumatoid arthritis
   i. Osteoarthritis of the affected elbow
   j. Fibromyalgia judged sufficient to compromise the evaluation of response
7. Subject currently has an acute infection at the injection site.
8. History of lymphoproliferative disease or any known malignancy or history of malignancy of any organ system (except for basal cell carcinoma, squamous cell carcinoma or actinic keratoses of the skin that have been treated with no evidence of recurrence in the past 5 years, carcinoma in situ of the cervix or non-invasive malignant colon polyps that have been removed), treated or untreated within the past 5 years prior to baseline regardless of whether there is evidence of local recurrence or metastases.
9. Subject is physically or mentally compromised (e.g., currently being treated for a psychiatric disorder, senile dementia, Alzheimer's disease, etc) to the extent that the Investigator judges the subject to be unable or unlikely to remain compliant.
10. Subject has received an investigational therapy or approved therapy for investigational use within 30 days of injection procedure or during the follow-up phase of the study.

Subjects should abstain from strenuous exercise for 48 hours prior to each visit, and while in the CRU.

Subjects are also not allowed to operate any motor vehicle for at least 24 hours after injection of the investigational medicinal product (IMP).

The subjects were required to adhere to the following restrictions:
Subjects should abstain from alcohol for 48 hours prior to each visit until after discharge from the clinical research unit (CRU).
Subjects should abstain from tobacco and nicotine-containing products (including e-cigarettes) for 90 days prior to dosing, throughout the study until completion of the study.
Subjects should refrain from consuming poppy seeds 48 hours prior to screening and each visit to avoid a positive result on the drugs of abuse screen.

Subjects were fasted overnight for 8 hours pre-dose. In addition, subjects are advised to fast at least 4 hours prior to any visit where clinical laboratory evaluations were carried out.

The ultrasound assessment at screening was conducted at any time during the 4-week screening period and includes a scan of the contra-lateral elbow. At least 1 week prior to dosing, subjects were required to have discontinued any use of opiate or NSAID medications they have been using for pain management. From Day −7 to Day −1, subjects were allowed to take paracetamol (less than or equal to 4 g/day) or use an ice pack to manage pain.

Subjects attended the CRU on Day −2/−1 and pre-dose on Day 1 to confirm eligibility and undergo safety and efficacy baseline assessments. Following confirmation of eligibility, subjects were randomised and dosed with CWT-001 or placebo. After the injection, the subject was rested for 30 minutes. Subjects underwent post-dose safety and PK assessments and were discharged from the CRU on the same day. Subjects returned to the CRU on Day 2 for PK sampling assessments, and for safety and PK assessments on Day 7. Subjects returned to the CRU on Day 14, Day 28 and Day 90 for safety and efficacy assessments (further details provided below). Subjects underwent ultrasound assessment on Day 28 and Day 90. The duration of the study for each subject is approximately 16 weeks.

The impact of tendinopathy on various aspects of patient health related quality of life were assessed by the following instruments which are accepted, validated patient reported outcome tools in upper limb tendinopathy (ref. 16): subjects' assessment of tendinopathy pain intensity (VAS), QuickDASH score, ASES-E score, PRTEE score, and ultrasound assessment of tendon at days 28 and 90 post-injection.

Stopping criteria include:
A ‘serious’ adverse reaction in 1 subject, i.e., a serious adverse event (SAE, defined below) that is considered at least possibly related to IMP administration.
‘Severe’ non-serious adverse reaction, i.e., a severe non-serious AE considered as at least possibly related to IMP administration, in 2 subjects in the same cohort, independent of within, or not within, the same system-organ class (SOC).
If there is an unacceptable tolerability profile based on the nature, frequency and intensity of observed AEs and/or clinical safety monitoring.

An AE is any untoward medical occurrence in a study subject which either emerges, or worsens from screening, during the clinical study, regardless of its potential relationship to the medicinal product.

An AE, therefore, can be any unfavourable or unintended sign, including a clinically-significant abnormal laboratory finding, symptom, or disease.

An SAE is an untoward medical occurrence that, at any dose:
Results in death,
Is life-threatening,
   Note: The term ‘life-threatening’ in the definition of ‘serious’ refers to an event in which the subject was at risk of death at the time of the event. It does not refer to an event, which hypothetically might have caused death, if it were more severe.
Requires hospitalisation or prolongation of existing hospitalisation,
   Note: In general, hospitalisation signifies that the subject has been detained (usually involving at least an overnight stay) at the hospital or emergency ward for observation and/or treatment that would not have been appropriate in the physician's office or out setting. Complications that occur during hospitalisation are AEs. If complication prolongs hospitalisation or fulfils any other serious criteria, the event is serious. When in doubt whether ‘hospitalisation’ occurred or was necessary, the AE should be considered serious.
Results in persistent or significant disability or incapacity.
   Note: The term ‘disability’ means a substantial disruption of a person's ability to conduct normal life functions. This definition is not intended to include experiences of relatively minor medical significance such as uncomplicated headache, nausea, vomiting, diarrhoea, influenza and accidental trauma (e.g., sprained ankle) which may interfere or prevent everyday life functions but do not constitute a substantial disruption.
Is a congenital abnormality or birth defect,
Is an important medical event.
   Note: Medical or scientific judgement should be exercised in deciding whether reporting is appropriate in other situations, such as important medical events that may not be immediately life threatening, or result in death or hospitalisation, but may jeopardise the subject or may require medical or surgical intervention to prevent one of the other outcomes listed in the above definition. These should also be considered serious. Examples of such events are invasive or malignant cancers, intensive treatment in an emergency room or at home for allergic bronchospasm, blood dyscrasias or convulsions that do not result in hospitalisation, or development of drug dependency or drug abuse.
   Note: The terms “serious” and “severe” are not synonymous. The term “severe” is often used to describe the intensity (severity) of a specific event (as in mild, moderate or severe myocardial infarction); the event itself, however, may be of relatively minor medical significance (such as severe headache). This is not the same as “serious”, which is based on subject/event outcome or action criteria described above and are usually associated with events that pose a threat to a subject's life or functioning. A severe AE does not necessarily need to be considered serious. For example, persistent nausea of several hours duration may be considered severe nausea but not an SAE. On the other hand, a stroke resulting in only a minor degree of disability may be considered mild but would be defined as an SAE based on the above criteria. Seriousness (not severity) serves as a guide for defining regulatory reporting obligations None of the subjects met any of the stopping criteria during the study.

### Pharmacokinetic Assessments

Pharmacokinetics for CWT-001 was assessed by measuring serial plasma concentrations of miR29a.

A baseline PK blood sample is collected within 30 minutes pre-dose. Blood samples for determination of plasma concentrations of miR29a were taken at the timepoints stated in Table 9 below. Plasma concentration-time data are analysed by noncompartmental methods. Nominal times were used for interim decisions, however actual sampling times were used for final calculations. Approximate sampling volumes are provided in Table 8.

TABLE 8
Sampling summary

| Sample | Sample | Number of Samples | Total |
|---|---|---|---|
| Type | Purpose | (Volume Per Sample) | Volume (mL) |
| Blood | Haematology | 5 (2.0 mL) | 10.0 mL |
| Blood | Biochemistry | 5 (5.0 mL) | 25.0 mL |
| Blood | Coagulation | 5 (4.5 mL) | 22.5 mL |
| Blood | Pharmacokinetic | 12 (3.0 mL) | 36.0 mL |
| | Total Blood: | | 93.5 mL |

Additional samples were drawn if needed for safety reasons. Up to 2 additional pharmacokinetic samples may be taken if needed, in order to define the pharmacokinetic profile.

Pharmacokinetic plasma samples are collected and miR29a is quantified using a validated quantitative real time PCR method developed at Thermo Fisher.

TABLE 9
Schedule of Assessments

| | Screening | | Treatment Period | | | | FU | EOS |
|---|---|---|---|---|---|---|---|---|
| | −4 Weeks | −1 Week | Week 1 | | | Week 2 | Week 4^{a} | Week 13 |
| Schedule of | Day −28 to | Day −2/ | | | Day 7 | Day 14 | Day 28 | Day 90 |
| Assessments | Day −3 | Day −1 | Day 1 | Day 2 | (±1 day) | (±2 days) | (±2 days) | (±2 days) |

Pharmacokinetic Assessments

| | | | |
|---|---|---|---|
| Blood Sampling¹ | Predose, 0.5, 1, | 24 and | X |
| | 2, 3, 4, 6, 8, 12 | 36 hours | |
| | hours post-dose | post-dose | |

Efficacy Assessments

| | | | | | |
|---|---|---|---|---|---|
| Elbow Pain/Tendon | | X^{m} | X | X | X |
| Pain Questionnaire | | | | | |
| Disabilities of the | | X^{m} | X | X | X |
| Arm, Shoulder and | | | | | |
| Hand Questionnaire | | | | | |
| (DASH) | | | | | |
| American Should | | X^{m} | X | X | X |
| and Elbow | | | | | |
| Surgeons Elbow | | | | | |
| (ASES-E) | | | | | |
| Patient Rated | | X^{m} | X | X | X |
| Tennis Elbow | | | | | |
| Evaluation | | | | | |
| (PRTEE) | | | | | |
| Ultrasound | Xⁿ | | | X | X |
| Assessment of | | | | | |
| Lateral Elbow | | | | | |
| Tendons | | | | | |

Abbreviations: EOS—end-of-study; FU—follow-up; PK—pharmacokinetic.
^{a}The follow-up visit takes place 28 days after dosing, or at early discontinuation (if applicable).

### Efficacy Assessment—Visual Analogue Scale

The subject assessed his/her pain in a diary once per day for the first 14 days post treatment rating the pain during the previous 24 hours. After Day 14 the subject kept a weekly pain diary until day 90 post injection. The subject's assessment of pain was performed using 100 mm visual analogue scale (VAS) ranging from “no pain” to “unbearable pain” after the question “Please indicate with a vertical mark (|) through the horizontal line the most pain you had from your tendinopathy during the last 24 hours”. A VAS is a validated measurement instrument that tries to measure a characteristic or attitude that is believed to range across a continuum of values and cannot easily be directly measured. It is often used in epidemiologic and clinical research to measure the intensity or frequency of various symptoms (ref. 17).

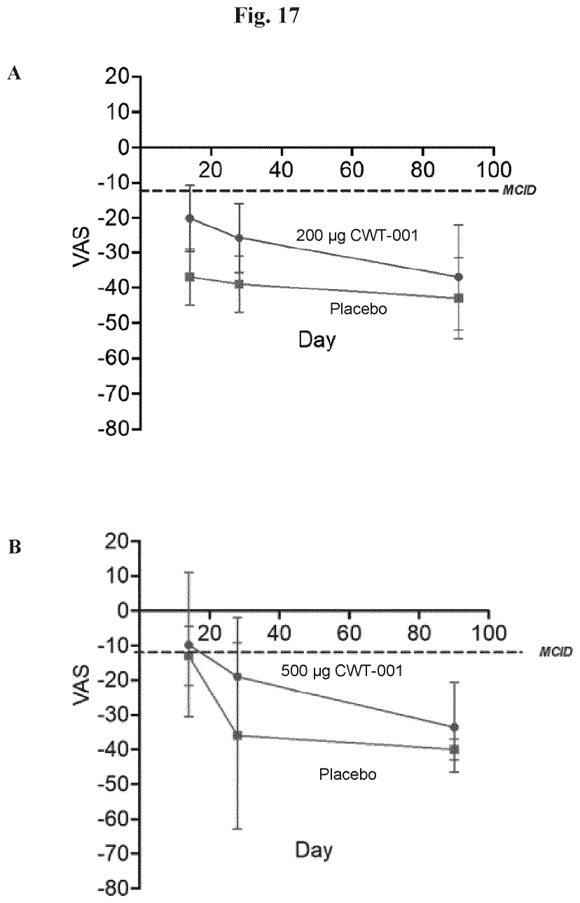

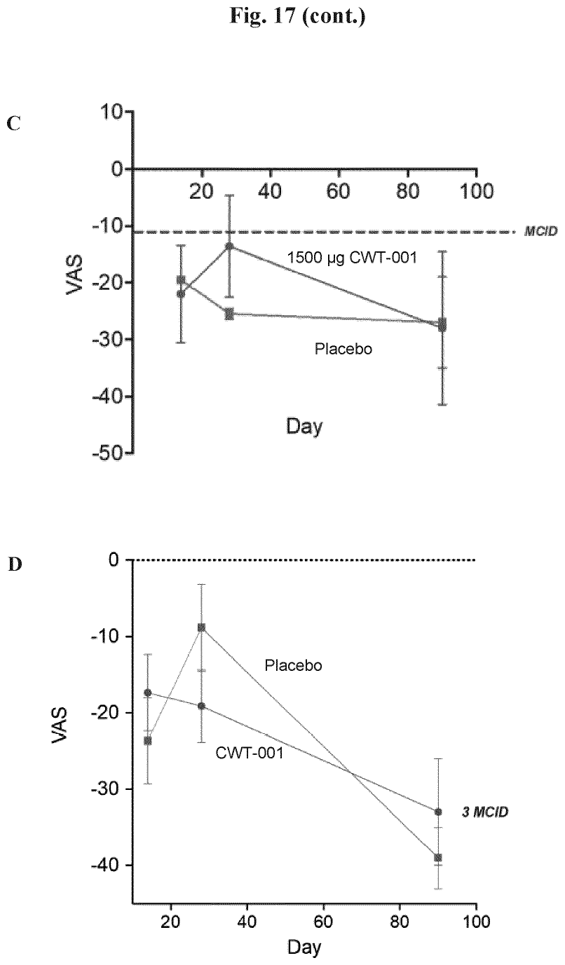

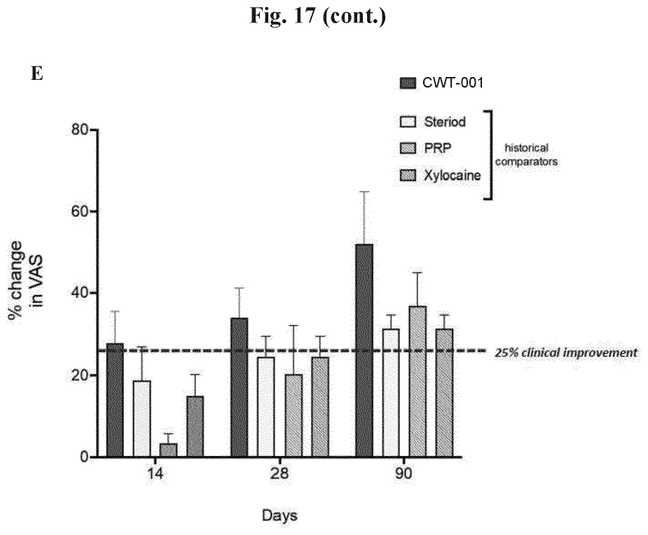

The results are shown in FIG. 17. A reduction in VAS score was observed for all groups of subjects, those that received a 200 μg (FIG. 17A), 500 μg (FIG. 17B), or 1500 μg (FIG. 17C) dose of CWT-001 and those that received a placebo saline injection. These reductions in VAS score generally exceeded the minimal clinically importance difference (MCID) for VAS in the treatment of elbow pain, which is −11 (ref. 18). The mean change in VAS score for all subjects that received any dose of CWT-001 was determined and compared with placebo (FIG. 17D). Treatment with CWT-001 resulted in a change of VAS score that exceeded the change observed with the placebo at day 28. The mean change in VAS score observed was −33±7 for CWT-001-treated subjects at day 90, which corresponds to a change of 3 MCID.

Randomized controlled trials (RCTs) in orthopaedics have evaluated the use of normal saline (NS) injections as a placebo/control group, expecting no physiological effect of this substance. However, as suggested by current best evidence within the field (refs. 18, 19, 20, 21), questions arise as to whether NS can act as an appropriate placebo in this field. For example, a recent study (ref. 22) of intra-articular saline injections into knee joints in patients suffering from osteoarthritis suggested plausible and potential beneficial effects of saline such as pain relief and osmolality-related changes. From a molecular and physiological standpoint, the contribution of hypertonic saline administration in reducing proinflammatory cytokines (RANTES, MCP-1, IP-10, G-CSF) after induction with TNF-α and IL-1b and how sodium ions and osmolality can contribute to osteoarthritis pathology, thus suggesting it is a potent anti-inflammatory mediator. There is a known placebo effect of a 70% reduction in VAS scores in tendinopathy clinical trials where an intra-tendon injection of saline is used as the control (ref. 18).

A recent systemic review of NS as placebo specifically in lateral epicondylitis trials (ref. 23) found that in all but one RCT examined, there was no statistically significant difference in Patient Reported Outcome Measures (PROMS) between saline and non-saline (platelet-rich plasma (PRP), autologus conditioned plasma (ACP), corticosteroid and botulinum toxin) injection, with NS exerting similar therapeutic effects as the injectable therapies being tested. Based on this work it was proposed within the field that owing to the lack of robust evidence to elucidate the principles, physiological characteristics, effects, and underlying mechanisms of NS injections, alternative control methods, such as sham syringes/needles, could be performed as better study control arms (such as that described in Boesen et al., 2017; ref. 24).

In light of the known problems associated with the use of intra-tendon saline injections as a placebo in tendinopathy studies, the mean percentage change in VAS score for all subjects that received any dose of CWT-001 was additionally compared with the mean percentage change in VAS score for subjects that received alternative treatments for lateral epicondylitis in a separate study (FIG. 17E; ref. 25). This study found that corticosteroid, platelet rich plasma (PRP) and xylocaine were all safe and effective in the treatment of lateral epicondylitis. The percentage change in VAS observed for treatment with CWT-001 in the present study exceeded the percentage change in VAS observed for each of the known treatments at all time points.

### Efficacy Assessment—ASES-E Score

The ASES score is developed by the Society of the American Shoulder and Elbow Surgeons for the assessment of shoulder and elbow function (ref. 26). The ASES-E score is self-administered and has 17 questions in the areas of symptoms and functions. The severity of pain was graded on a 10 cm visual analogue scale that ranges from 0 (no pain at all) to 10 (pain as bad as it can be). The recall period is how does the subject feel at this point in time. This validated tool has been chosen as it combines subject reported outcomes with a physician assessment in change in elbow range of movement and strength (ref. 27).

Efficacy Assessment—Quick Disability of the Arm, should, and Hand (DASH) Score

The Quick DASH is an abbreviated form of DASH, a subject reported outcome tool, developed by the American Academy of Orthopaedic Surgeons along with the Institute for Work & Health (Toronto, Ontario, Canada). The Quick DASH Index is self-administered and uses 11 items to measure physical function and symptoms in subjects with any or multiple musculoskeletal disorders of the upper limb. This validated outcome measure (ref. 27) provides core information of the change in subject functionality post CWT-001 injection.

Each item of the QuickDASH has five response options: 1=no difficulty; 2=mild difficulty; 3=moderate difficulty; 4=severe difficulty; 5=unable.

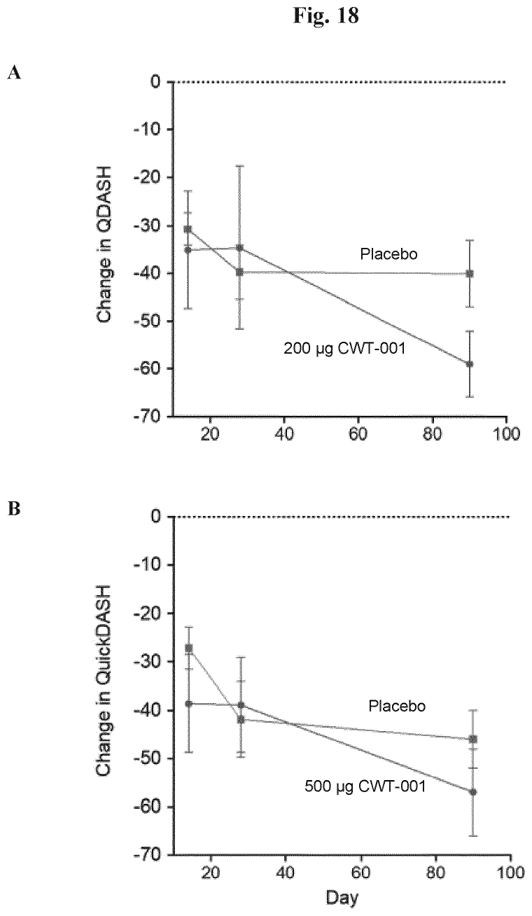

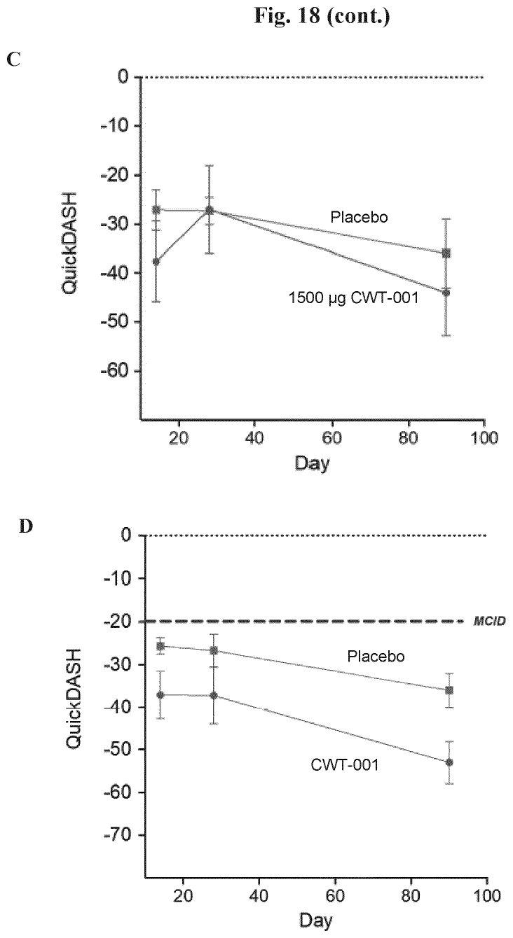

The results are shown in FIG. 18. A reduction in QuickDASH score was observed for subjects that received a 200 μg (FIG. 18A), 500 μg (FIG. 18B), or 1500 μg (FIG. 18C) dose of CWT-001. At 90 days, the reduction in QuickDASH score was greater for subjects that had received CWT-001 than for subjects that had received a placebo saline injection for each dosage cohort. The mean change in QuickDASH score for all subjects that received any dose of CWT-001 was determined and compared with placebo (FIG. 18D). Treatment with CWT-001 resulted in a change of QuickDASH score that exceeded the change observed with the placebo at all time points. The mean change in QuickDASH score for subjects that had received any dose of CWT-001 also exceeded the established MCID for DASH in patients with upper-limb musculoskeletal disorders, which is −20 points (ref. 28). The mean improvement in QuickDASH score at Day 90 relative to the pre-dose score for subjects that had received any dose of CWT-001 was −53±5 points.

### Efficacy Assessment—Patient Rated Tennis Elbow Evaluation (PRTEE)

The PRTEE formerly called the Patient-Rated Forearm Evaluation Questionnaire, is a 15-item self-reported questionnaire to measure perceived pain and disability in people with tennis elbow. It has three subscales: pain, usual activities and specific activities. The pain subscale has five items about the intensity of pain during various activities. The specific activities subscale has six items tapping into the difficulty experienced while performing specific activities, like lifting a coffee cup. The four items in the ‘usual activities’ subscale capture the difficulty experienced in performing usual daily roles including those performed during work and recreation. The PRTEE is an excellent example of a disease-specific self-report measure. This is very helpful in capturing aspects of pain and function that are more specific to tennis elbow than a generic joint-specific or region-specific measure. This validated outcome measure (ref. 27) provides core information of the change in subject functionality post CWT-001 injection.

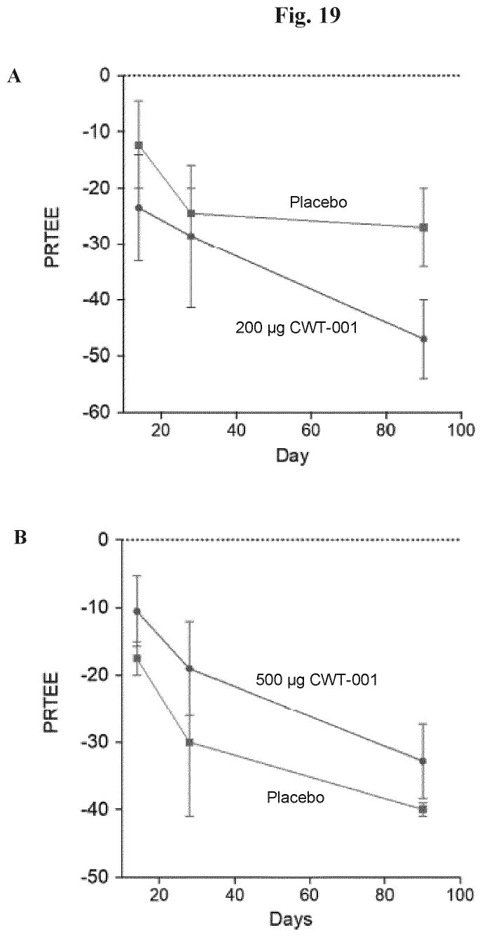

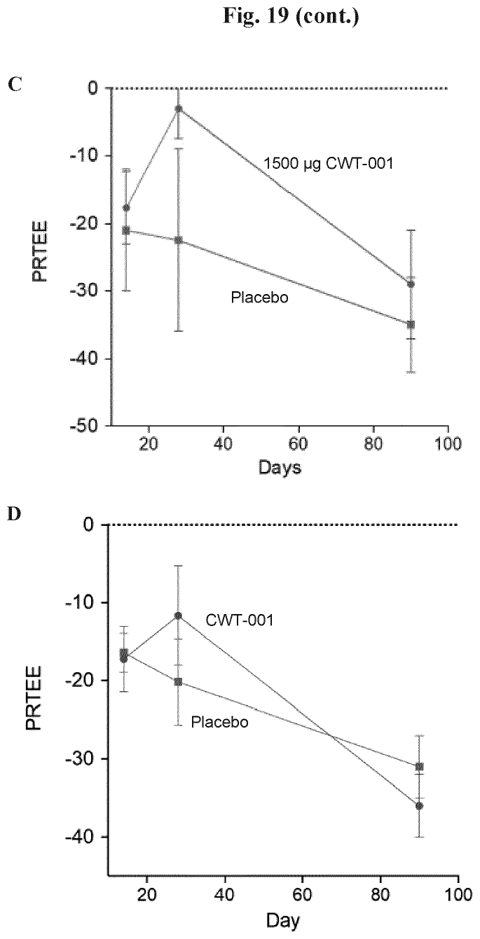

The results are shown in FIG. 19. A reduction in PRTEE score was observed for subjects that received a 200 μg (FIG. 19A), 500 μg (FIG. 19B), or 1500 μg (FIG. 19C) dose of CWT-001. The reduction in PRTEE score observed for subjects that received a 200 μg dose of CWT-001 exceeded the reduction in PRTEE score for subjects that received a placebo at all time points.

The mean change in PRTEE score for all subjects that received any dose of CWT-001 was determined and compared with placebo (FIG. 19D). The mean improvement in PRTEE score at Day 90 was 71±6% of the baseline pre-dose score for subjects that had received any dose of CWT-001. This exceeds the MCID for clinical significance defined as “much better” or “completely recovered” for PRTEE, which is an improvement of 37% of baseline score (ref. 29)

### Efficacy Assessment —Ultrasound Assessment

The lateral tendon complex at the level of the elbow is easily accessible to ultrasonographic examination because it is located immediately under the subcutaneous tissue and the fascia. Normally, the complex is visualized as a hyperechoic linear structure aligned between the corresponding attachments. Characteristic ultrasound finding of lateral elbow tendinopathy include focal hypoechoic areas within the tendon, which are associated with loss of the normal internal collagen fibre alignment pattern. Importantly studies have shown that sonographic features are consistent with disease process and demonstrate resolution with improvement of symptoms using VAS and DASH scores post therapeutic treatment (ref. 30). Based on preclinical models, ultrasound assessment was conducted at Days 28 and 90 post-injection. Importantly, this provides data on the mechanism of action of CWT-001 (improvement in collagen structure), return to normal tendon structure post treatment which is beneficial for subject recovery and durability of the treatment.

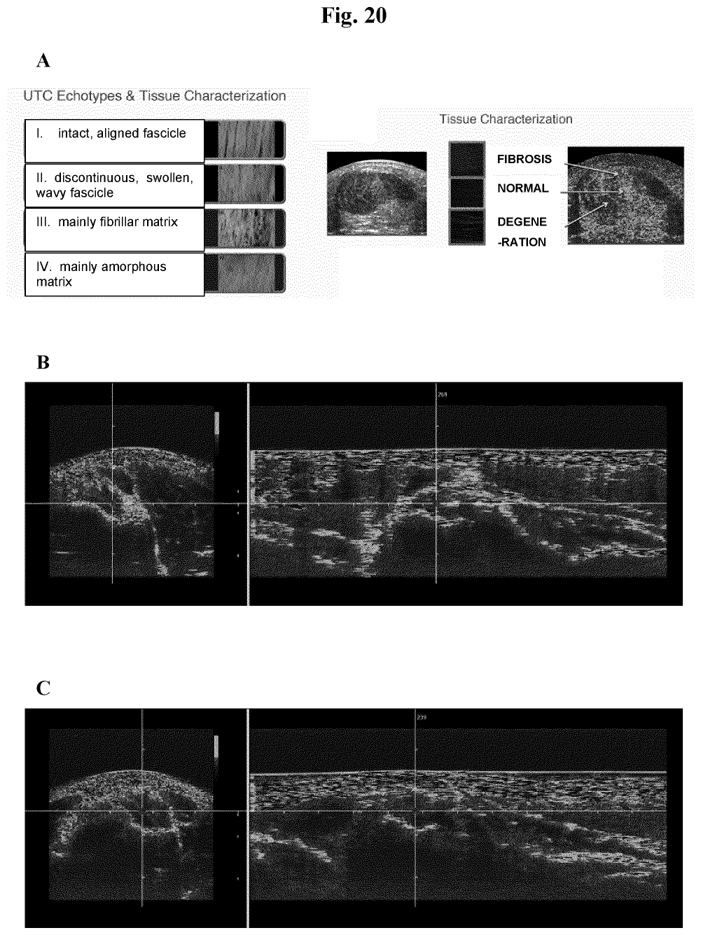

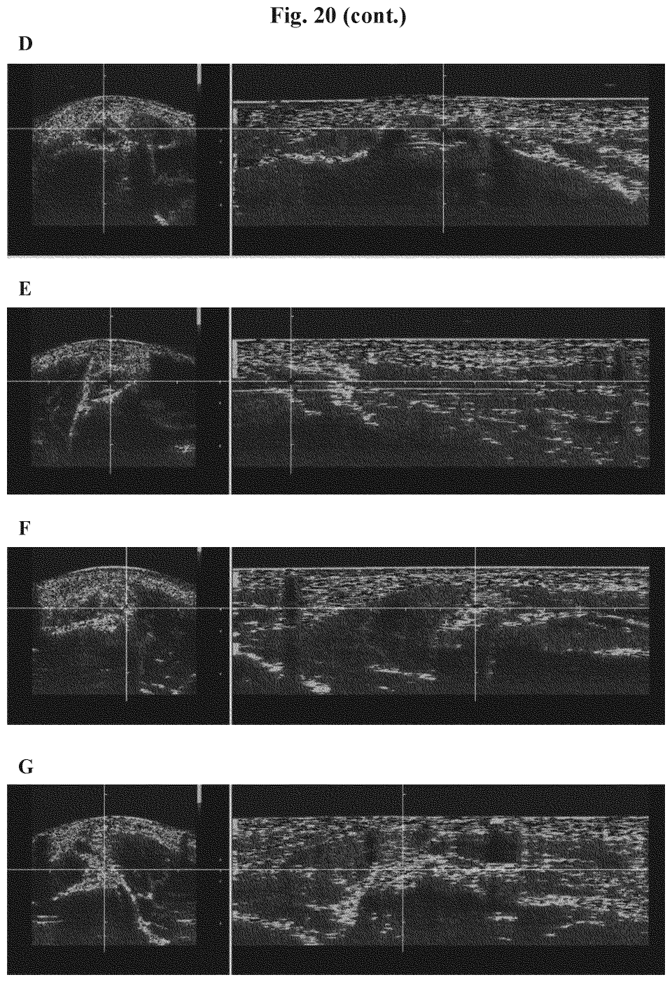

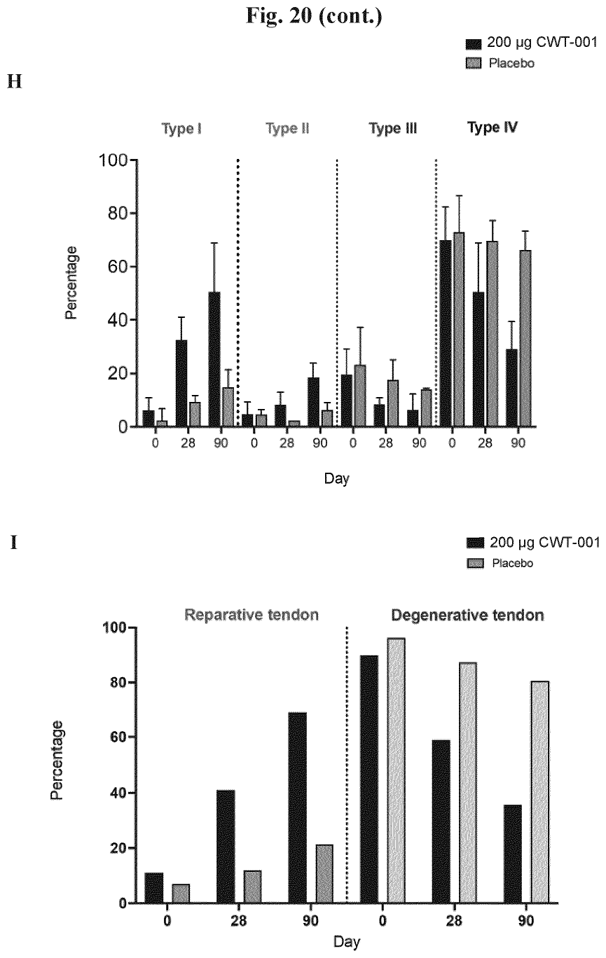

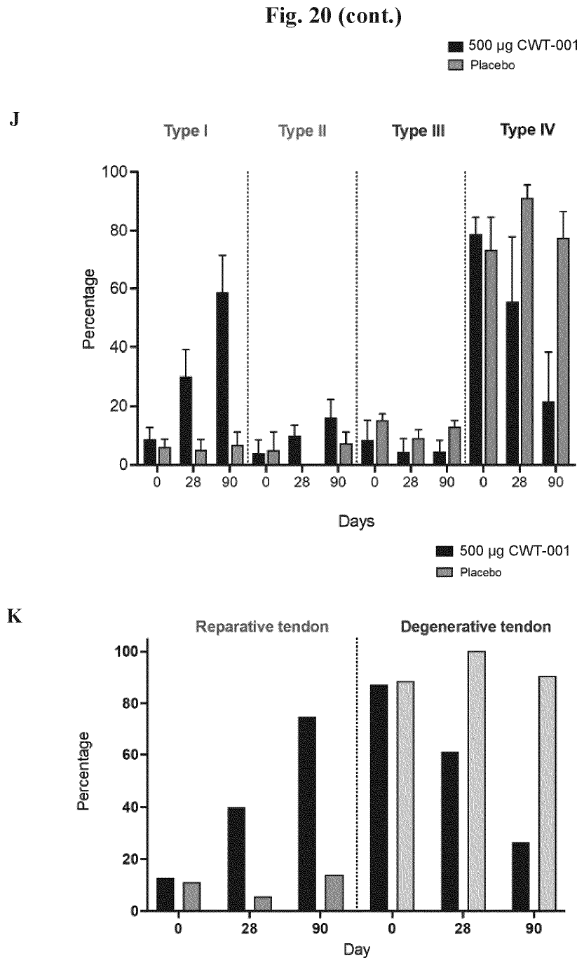

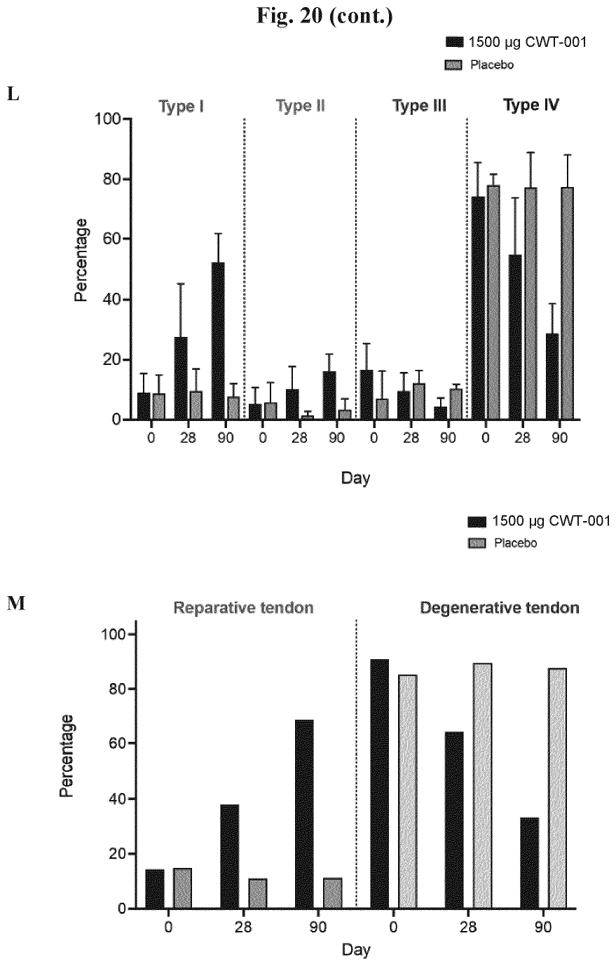

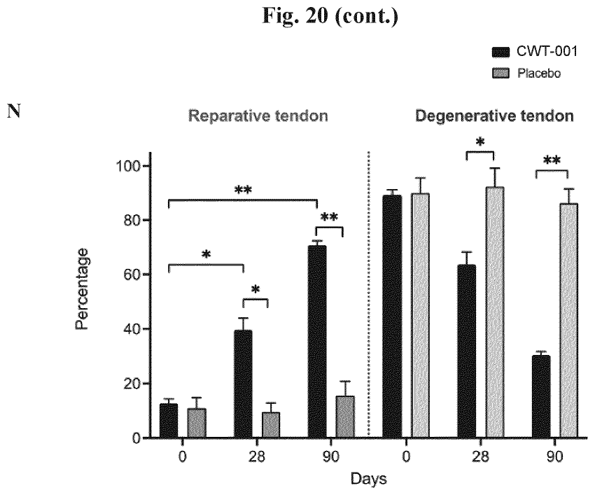

An imaging modality called ultrasound tissue characterisation (UTC) can be used to visualise tendon structure and to quantify tendon matrix integrity (ref. 31). Unlike two-dimensional ultrasound and colour Doppler, UTC objectively quantifies grey-scale tendon matrix changes into 4 different echotypes related to tendon integrity. Types I (green) and II (blue) represent an organised matrix; Types III (red) and IV (black) represent a disorganised matrix (FIG. 20A). The advantage of this tool, compared to conventional ultrasound, is that it captures a three-dimensional ultrasound image of the tendon and uses standardised parameters (transducer tilt angle, depth and gain settings). Bilateral scans (both lateral elbows) were performed by a single examiner with experience performing UTC scans. Images were acquired using a 7-10 MHz linear ultrasound transducer (SmartProbe 12L5-V, Terason 2000+; Teratech; Burlington, MD, USA) positioned in a tracking device (UTC Tracker, UTC Imaging, Stein, The Netherlands) that moves automatically along the tendon long axis over a distance of 5 cm recording regular images at intervals of 0.2 mm. Transducer tilt, angle, gain, focus and depth are standardised by the tracking device. Images from the sagittal, coronal and transverse planes were compiled to create a three-dimensional view of the tendon. Ultrasound tissue characterisation assessment occurred pre-treatment (within 4 weeks) and at Days 28 and 90.

Representative ultrasound images of the lateral elbow are provided for a subject that had received the placebo at pre-injection (FIG. 20B), Day 28 (FIG. 20C) and Day 90 (FIG. 20D), and for a subject that had received 200 μg CWT-001 at pre-injection (FIG. 20E), Day 20 (FIG. 20F) and Day 90 (FIG. 20G).

The percentages of UTC Type I, II, III and IV tendon tissue and the total percentage of reparative tendon (UTC Types I and II) vs degenerative tendon (UTC types III and IV) were quantified for the subjects. For each dosage cohort, the percentage of reparative tendon tissue, in particular intact UTC type I tendon, increased from Day 0, to Day 28 to Day 90 for subjects that received CWT-001, and was much higher for subjects that received CWT-001 than for subjects that received the placebo. There was a corresponding reduction in degenerative tendon, in particular the most severely damaged UTC type IV tendon over the study duration and relative to placebo group (see FIG. 20H, I for 200 μg CWT-001, FIG. 20J,K for μg CWT-001 and FIG. 20L,M for 1500 μg CWT-001).

The mean percentages of reparative tendon for all subjects that received any dose of CWT-001 increased significantly from Day 0 to Day 28 (P<0.05) and to Day 90 (P<0.01), while no significant change was observed for the subjects that received the placebo. The subjects that had received CWT-001 also had significantly higher levels of reparative tendon than the subjects that had received the placebo at Day 28 (P<0.05) and Day 90 (P<0.01), and had significantly lower levels of degenerative tendon at Day 28 (P<0.05) and Day 90 (P<0.01).

Thus, the UTC data demonstrate a quantifiable improvement in tendon structure for human subjects with lateral epicondylitis following treatment with a single 200 μg, 500 μg or 1500 μg dose of CWT-001.

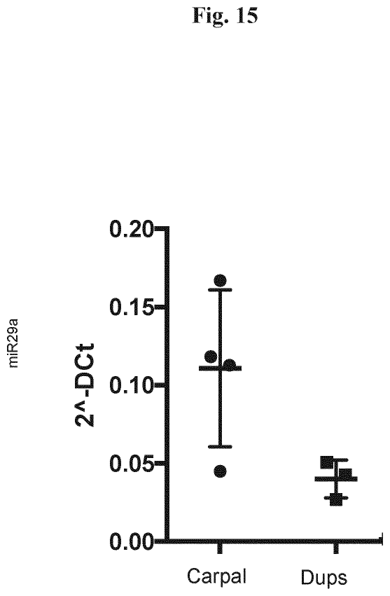

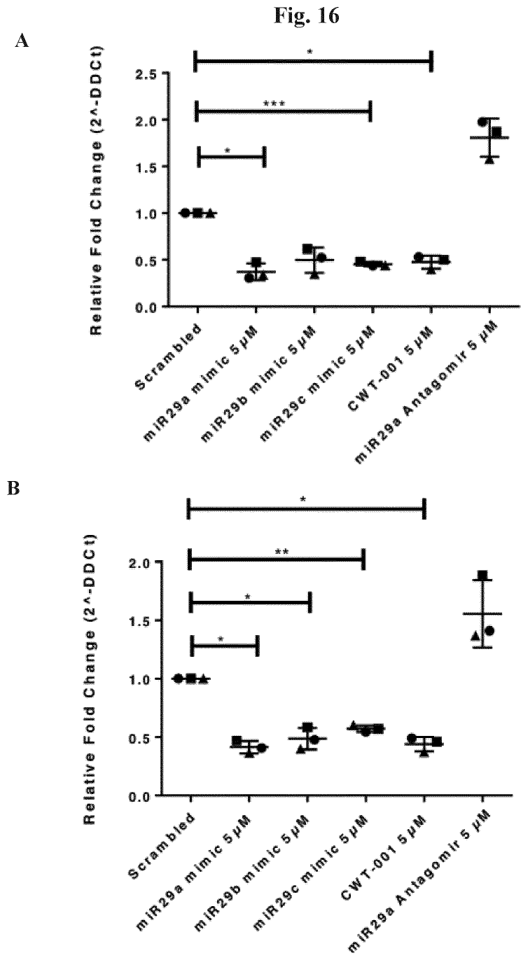

### Example 8—Dupuvtren's Disease (FIGS. 15 and 16)

qPCR analysis of miR29a expression in healthy and Dupuytren's fascia tissue was performed (4 samples for the healthy group, 3 samples for the Dupuytren's group). The results showed that the levels of miR29a were significantly reduced in samples from Dupuytren's tissue relative to healthy controls undergoing carpal tunnel release surgery (FIG. 15). These experiments showed that the fibrotic growth observed in Dupuytren's diseases is associated with a reduction in miR29a expression. This observation indicated that miR-29 replacement therapy may prevent or reduce the collagen overproduction seen in Dupuytren's diseases.

To investigate further whether miR-29 replacement therapy could be useful in the treatment of Dupuytren's disease, cultured Dupuytren's fibroblasts were treated with 5 μM of a miR-29a mimic, a miR-29b mimic, a miR-29c mimic, CWT-001, a miR-29a antagomir, or a scrambled miR-29a control. Levels of collagen 1 and collagen 3 expression following treatment were analysed by qPCR.

The results showed that expression of both collagen 1 (FIG. 16A) and collagen 3 (FIG. 16B) was significantly reduced following treatment with the miR-29a mimic, miR-29b mimic, miR-29c mimic or CWT-001, relative to treatment with a scrambled miR-29 control. These data show that treatment with a miR-29 mimetic compound such as CWT-001 may be effective in the treatment of Dupuytren's diseases.

### Example 9—Peyronie's Disease

CWT-001 is administered to subjects with Peyronie's Disease as a single intra-lesional (intra-penile) injection into the tunica albuginea of the penis. Subjects are males with early stage (acute or active phase) Peyronie's Disease, with a penis curvature of <300 and/or palpable plaque <2 cm in extent (based on Kelami classification, ref. 32). Subjects are followed up for 1 year post-injection. Efficacy assessment includes curvature of the penis, penile duplex Doppler ultrasound, pain VAS, IEFF questionnaire, and PDQ questionnaire.

It will be understood that the invention has been described by way of example only and modifications may be made whilst remaining within the scope and spirit of the invention.

### REFERENCES

[1] Millar et al. (2015) Nature Communication. 6:6674
[2] WO 2015/107340
[3] Watts et al. (2017) Mol Ther. 25(10): 2415-2426
[4] Maurer et al. (2010) Arthritis Rheum. 62(6):1733-43
[5] Fabbri et al. (2012) PNAS 109 (31) E2110-E2116
[6] Menzel et al. (1979) Hand 11, 243-248
[7] Tubiana et al. (1968) Surgical Clinics of North America, 48(5): 979-984
[8] Castiglione et al. (2019) Sex Med. Mar; 7(1): 94-103
[9] Ralph et al. (1997) J Urol Jan; 157(1):291-294
[10] Somers et al. (1989) J Urol 141, 629-631
[11] Nagraba et al. (2013) Ortop Traumatol Rehabil. 15(2): 109-116
[12] Lake et al. (2008) Disabil Rehabil 30:1530-1541
[13] Lindow et al. (2012) Nat Biotechnol. 30(10):920-3
[14] Robbins et al. (2009) Oligonucleotides 19: 89-102
[15] Sorani & Campbell (2016) Br J Radiol. 89(1057); 20150368
[16] Macdermid et al. (2015) J Orthop Sports Phys Ther. 45(11): 950-964
[17] Hjermstad et al. (2011) J Pain Symptom Manage. 41(6): 1073-1093
[18] Acosta-Olivo et al. (2020) Am J Sport Med. 48(12):3094-3102
[19] Previtali et al. (2020) Cartilage 18:1-12
[20] Wolf et al. (2011) J Hand Surg Am 36(8):1269-72
[21] Gazendam et al. (2021) Brit J Sport Med. 55:256-261
[22] Bar-Or et al. (2017) Open Rheumatol J. 31; 11:16-22
[23] Gao et al. (2019) Athroscopy. 35(6):1847-1859
[24] Boesen et al. (2017) Am J Sports Med. 45(9):2034-2043
[25] Reddy et al. (2016) J Trauma Treat 5:304
[26] Richards et al. (1994) J Shoulder Elbow Surg. 3(6): 347-352
[27] Longo et al. (2008) Br Med Bull. 87: 131-161
[28] Franchignoni et al. (2014) J Orthop Sport Phys. 44(1): 30-9
[29] Poltawski et al. (2011) Hand Therapy. 16(3):52-57
[30] Clarke et al. (2010) Am J Sports Med. 38(6): 1209-1214
[31] van Schie et al. (2010) Br J Sports Med. 44(16): 1153-1159
[32] Kelimi (1983) Urol Int. 38(4):229-233

1. A miR-29 compound or a salt thereof, comprising:
(a) a guide strand comprising the nucleobase sequence 5′-UAGCACCAUCUGAAAUCGGUUA-3′ (SEQ ID NO: 1), or a nucleobase sequence which differs from SEQ ID NO: 1 at no more than three positions outside of the sequence 5′-AGCACCA-3′ (SEQ ID NO: 8); wherein at least 50% of the nucleosides comprise a 2′-fluoro ribose and wherein one or two nucleosides at the 3′ end each comprise a 2′-O-methyl ribose; and
(b) a passenger strand, comprising the nucleobase sequence 5′-ACUGAUUUCUUUUGGUGUUCAG-3′ (SEQ ID NO: 2) or 5′-ACUGAUUUCUUUUGGUGUUCA-3′ (SEQ ID NO: 3), or comprising a nucleobase sequence that differs from SEQ ID NO: 2 at no more than three positions; and wherein at least 10 contiguous nucleosides are alternately a 2′-O-methyl ribose nucleoside and a nucleoside with an unmodified ribose moiety.

2. The miR-29 compound or salt of claim 1, wherein one or two nucleosides at the 3′ end of the guide strand each comprise a 2′-O-methyl ribose and: (i) each remaining nucleoside of the guide strand that comprises a 2′-modified ribose is a 2′-fluoro ribose nucleoside, or (ii) each remaining nucleoside of the guide strand is a 2′-fluoro ribose nucleoside or an unmodified ribose moiety.

3. The miR-29 compound or salt of claim 1, wherein (i) each nucleoside of the guide strand comprises a 2′-modified ribose, (ii) each nucleoside of the guide strand comprises a 2′-O-methyl ribose or a 2′-fluoro ribose, or (iii) one or two nucleosides at the 3′ end of the guide strand each comprise a 2′-O-methyl ribose and each remaining nucleoside of the guide strand is a 2′-fluoro ribose nucleoside.

4. The miR-29 compound or salt of claim 1, wherein all of the internucleoside linkages of the guide strand and/or the passenger strand are phosphodiester internucleoside linkages.

5. The miR-29 compound or salt of claim 1, wherein the nucleobase sequence of the guide strand is as defined in SEQ ID NO: 1, and/or the nucleobase sequence of the passenger strand is as defined in SEQ ID NO: 2 or SEQ ID NO: 3.

6. The miR-29 compound or salt of claim 1, wherein the guide strand is 5′-fUfAfGfCfAfCfCfAfUfCfUfGfAfAfAfUfCfGfGfUmUmA-3′; and/or the passenger is 5′-mArCmUrGmArUmUrUmCrUmUrUmUrGmGrUmGrUmUrCdA-3′.

7. The miR-29 compound or salt of claim 1, further comprising a cholesterol moiety covalently attached to the 3′ end of the passenger strand.

8. A pharmaceutical composition comprising the miR-29 compound or salt thereof according to claim 1, and a pharmaceutically acceptable carrier or diluent.

9. The miR-29 compound or salt of claim 1, for use in therapy.

10. The miR-29 compound or salt of claim 1, for use in treating tendon damage or tissue fibrosis.

11. The miR-29 compound or salt of claim 1, for use in treating tendon damage, Peyronie's disease, or Dupuytren's disease.

12. A method for treating tendon damage, Peyronie's disease, OR Dupuytren's disease in a subject, comprising a single administration by injection of a dose of at least 47 μg of a miR-29 compound, or the equivalent dose of the salt thereof, into the damaged tendon, the tunica albuginea, or the palmar fascia of the subject.

13. (canceled)

14. (canceled)

15. The method of claim 12, wherein the dose of the miR-29 compound is at least 95 μg, at least 190 μg, at least 475 μg, at least 1420 μg, at least 1900 μg, at least 4270 μg, or the equivalent dose of the salt thereof.

16. The method of claim 12, wherein the dose of the miR-29 compound is no more than 4740 μg, or the equivalent dose of the salt thereof.

17. The method of claim 12, wherein the miR-29 compound or salt thereof is administered in a volume of about 1 ml of a sterile aqueous solution.

18. The method of claim 12, wherein the tendon damage is tendinopathy.

19. The method of claim 12, wherein the compound or salt thereof comprises:
(a) a guide strand comprising the nucleobase sequence 5′-UAGCACCAUCUGAAAUCGGUUA-3′ (SEQ ID NO: 1), or a nucleobase sequence which differs from SEQ ID NO: 1 at no more than three positions outside of the sequence 5′-AGCACCA-3′ (SEQ ID NO: 8): wherein at least 50% of the nucleosides comprise a 2′-fluoro ribose and wherein one or two nucleosides at the 3′ end each comprise a 2′-O-methyl ribose; and
(b) a passenger strand, comprising the nucleobase sequence 5′-ACUGAUUUCUUUUGGUGUUCAG-3′ (SEQ ID NO: 2) or 5′-ACUGAUUUCUUUUGGUGUUCA-3′ (SEQ ID NO: 3), or comprising a nucleobase sequence that differs from SEQ ID NO: 2 at no more than three positions; and wherein at least 10 contiguous nucleosides are alternately a 2′-O-methyl ribose nucleoside and a nucleoside with an unmodified ribose moiety.

20. The method of claim 12, wherein the guide strand of the miR-29 compound or salt thereof is
′-fUfAfGfCfAfCfCfAfUfCfUfGfAfAfAfUfCfGfGfUmUmA-3′, the passenger strand of the miR-29 compound or salt thereof is 5′-mArCmUrGmArUmUrUmCrUmUrUmUrGmGrUmGrUmUrCdA-3′ and comprises a cholesterol moiety covalently attached to the 3′ end of the passenger strand, and the dose of the miR-29 compound including the weight of the cholesterol moiety is between 50 μg and 5000 μg, for example 200 μg, 500 μg, 1500 μg or 4500 μg, or the equivalent dose of the salt thereof.


SEQUENCE LISTING (8 sequences)

SEQ ID NO: 1 - RNA, 22 nt, Artificial Sequence
  guide strand
uagcaccaucugaaaucgguua

SEQ ID NO: 2 - RNA, 22 nt, Artificial Sequence
  passenger strand
acugauuucuuuugguguucag

SEQ ID NO: 3 - RNA, 21 nt, Artificial Sequence
  passenger strand
acugauuucuuuugguguuca

SEQ ID NO: 4 - RNA, 21 nt, Artificial Sequence
  passenger strand
accgauuucaauuggugcuag

SEQ ID NO: 5 - RNA, 21 nt, Artificial Sequence
  passenger strand
accgauuucagauggugcuag

SEQ ID NO: 6 - RNA, 20 nt, Artificial Sequence
  passenger strand
accgauuucagauggugcua

SEQ ID NO: 7 - RNA, 22 nt, Artificial Sequence
  passenger strand
gacuugugguuuucuuuaguca

SEQ ID NO: 8 - RNA, 10 nt, Artificial Sequence
  seed sequence
  misc_feature (8)..(10): n is any nucleotide or is not required
agcaccannn